(19) United States
(12) Patent Application Publication
(10) Pub. No.: US 2004/0235849 A1
(43) Pub. Date: Nov. 25, 2004

(54) Tetrahydroquinoxalines acting as bradykinin antagonists

(76) Inventors: Bettina Beyreuther, Dusseldorf (DE); Michael Hahn, Langenfeld (DE); Christopher Kallus, Frankfurt (DE); Joachim Kruger, Dusseldorf (DE); Heinrich Meier, Wuppertal (DE); Elke Reibmuller, Wuppertal (DE); Leila Telan, Wuppertal (DE); Reilinde Nopper, Grenzach-Wyhlen (DE); Mathias Kroll, Essen (DE)

Correspondence Address: Jeffrey M Greenman, Bayer Corporation, 400 Morgan Lane, West Haven, CT, 06516, US

(21) Appl. No.: 10/483,464

(22) PCT Filed: Jul. 4, 2002
(86) PCT No.: PCT/EP02/07416

(30) Foreign Application Priority Data
Jul. 17, 2001 (DE) 10134721.9

(51) Int. Cl. A61K 31/498; C07D 241/36

(52) U.S. Cl. 514/249; 544/353

(57) ABSTRACT

The invention relates to novel tetrahydroquinoxalines and processes for their preparation, their use for the treatment and/or the prophylaxis of diseases, in particular for the treatment and/or prophylaxis of states of pain.

The invention relates to novel tetrahydroquinoxalines and processes for their preparation, their use for the treatment and/or prophylaxis of diseases, in particular for the treatment and/or prophylaxis of states of pain.

Kinins are peptides which are produced in the plasma (bradykinin) and peripheral tissue (kallidin) owing to injuries, inflammations, asthma and in anaphylactic and endotoxic shock. In addition to the important role played by kinins in cardiovascular homeostasis or the contraction and relaxation of smooth muscles (Bhoola al. Pharmacol. Rev. 1992, 44, 1-80), they result in particular in pain, inflammation and hyperalgesia. Since they in turn promote the production of other pain mediators such as prostaglandins, tachykinins and interleukins, there is a further potentiation of the pain response.

Kinins act via two Gq/11 protein-coupled 7 transmembrane receptor subtypes; whereas the bradykinin 2 receptor (B2-R) is activated by bradykinin and kallidin, the main fragments thereof, des-Arg9-bradykinin and des-Arg10-kallidin, are the preferred agonists for the bradykinin 1 receptor (B1-R). Receptor activation leads firstly to stimulation of phospholipase C and thus to release of intracellular calcium ions, secondly to activation of phospholipase A2 which opens ion channels by protein kinase C and thus brings about depolarization and excitation of the cell (Textbook of Pain, 4th edition; Wall and Melzack, Editors; Edinburgh, 1999, pages 61-62).

B1-R is, in contrast to B2-R, downregulated under physiological conditions, and is expressed and upregulated in cells through stimulation of disease-related mediators, e.g. interleukins. It therefore makes a contribution in particular to the chronic phase of the inflammatory response and to maintaining persistent hyperalgesia. In addition, B1-R is involved in central sensitization (Pesquero et al. Proc. Nat. Acad. Sci. USA, 2000, 97, 8140-8145) and in the modulation of spinal plasticity (Wotherspoon, G. and J. Winter Neurosci. Lett. 2000, 294, 175-178).

It is therefore sensible to use B1-R antagonists for the treatment of patients with inflammatory pain, neuropathic pain and (lower) back pain, pain associated with osteoarthritis, and pain associated with another etiology.

B1-R antagonists are also suitable for the treatment of asthma, diabetic vasculopathy, rhinitis, septic shock, atherosclerosis, multiple sclerosis or rheumatoid arthritis.

2-[3-Oxo-1-(phenylsulfonyl)-1,2,3,4-tetrahydro-2-quinoxalinyl]-N-phenylacetamide is disclosed in CAPLUS 1973, 136227, without stating a technical effect.

EP-A-0 509 398 and WO 00/00478 describe tetrahydroquinoxalines as HIV reverse transcriptase inhibitors for the treatment of viral diseases.

DE-A43 41 663 discloses tetrahydroquinoxalines as endothelin receptor antagonists for the treatment of, inter alia, migraine.

The present invention relates to compounds of the general formulae (I) and (Ia)

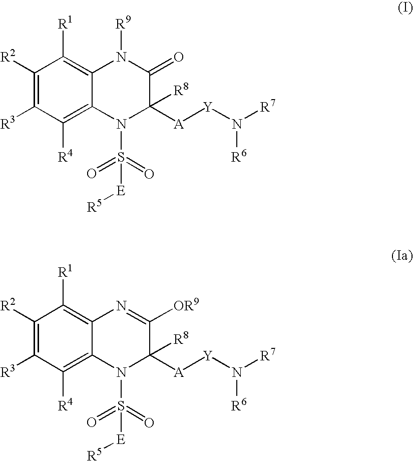

in which

A is (C₁-C₆)-alkanediyl,

E is a bond or (C₁-C₆)-alkanediyl,

Y is CO or SO₂,

R¹, R², R³ and R⁴ are identical or different and are hydrogen, halogen, trifluoromethyl, trifluoromethoxy, hydroxyl, nitro, cyano, amino, (C₁-C₆)-alkyl, (C₁-C₆)-alkoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl or carboxyl,

R⁵ is (C₆-C₁₀)-aryl or 5- to 10-membered heteroaryl, where aryl and heteroaryl are optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,

in which phenoxy, phenyl and 5- to 6-membered heteroaryl are in turn optionally substituted identically or differently by trifluoromethyl, (C₁-C₆)-alkyl, (C₁-C₆)-alkoxy or halogen,

R⁶ and R⁷ are identical or different and are hydrogen, (C₆-C₁₀)-aryl, 5- to 10-membered heteroaryl, 3- to 12-membered carbocyclyl, 4- to 12-membered heterocyclyl, or

are (C₁-C₁₀)-alkyl which is optionally substituted by halogen or a radical selected from the group of (C₁-C₆)-alkoxy, (C₆-C₁₀)-aryl, 5- to 10-membered heteroaryl, 3- to 12-membered carbocyclyl and 4- to 12-membered heterocyclyl,

where aryl, heteroaryl, heterocyclyl and carbocyclyl are optionally substituted identically or differently by radicals selected from the group of halogen, trifluoromethyl, trifluoromethoxy, hydroxyl, nitro, cyano, amino, (C₁-C₆)-alkyl, (C₃-C₈)-cycloalkyl, 5- to 7-membered heterocyclyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,

or

R⁶ and R⁷ together with the nitrogen atom form a 4- to 12-membered heterocyclyl radical which is bonded via nitrogen and which is optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, mono- or di-(C₁-C₆)-alkylamino, (C₁-C₆)-acyloxy, (C₁-C₆)-acyl, (C₁-C₆)-acylamino, (C₁-C₆)-alkoxycarbonyl, mono- or di-(C₁-C₆)-alkylaminocarbonyl, carbamoyl, carboxyl, (C₃-C₈)-cycloalkyl and phenyl,

where alkyl, cycloalkyl and phenyl in turn are optionally substituted identically or differently by one to three radicals selected from the group of halogen, phenyl, (C₁-C₆)-alkyl, (C₁-C₆)-alkoxy and (C₁-C₆)-alkylthio, in which phenyl in turn is optionally substituted identically or differently by radicals selected from the group of halogen or methyl,

R⁸ is hydrogen or (C₁-C₃)-alkyl which is optionally substituted by fluorine,

R⁹ is hydrogen or (C₁-C₆)-alkyl,

and the salts, hydrates and/or solvates thereof,

with the exception of 2-[3-oxo-1-(phenylsulfonyl)-1,2,3,4-tetrahydro-2-quinoxalinyl]-N-phenylacetamide.

The compounds of the invention may exist in stereoisomeric forms which either are related as image and mirror image (enantiomers) or which are not related as image and mirror image (diastereomers). The invention relates both to the enantiomers or diastereomers or respective mixtures thereof. These mixtures of enantiomers and diastereomers can be separated into the stereoisomerically pure constituents in a known mariner.

Salts preferred for the purposes of the invention are physiologically acceptable salts of the compounds of the invention.

Physiologically acceptable salts of the compounds of the invention may be acid addition salts of the compounds with mineral acids, carboxylic acids or sulfonic acids. Particularly preferred examples are salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

Salts which may also be mentioned, however, are salts with conventional bases such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procaine, dibenzylamine, N-methylmorpholine, dihydroabietylamine, 1-ephenamine or methylpiperidine.

Hydrates of the compounds of the invention are stoichiometric compositions of the compounds or its salts with water.

Solvates of the compounds of the invention are stoichiometric compositions of the compounds or its salts with solvent.

(C₁-C₆)-Acyl represents for the purposes of the invention a straight-chain or branched acyl radical having 1 to 6, preferably 1 to 4, carbon atoms. Preferred examples which may be mentioned are: acetyl, ethylcarbonyl, propylcarbonyl, isopropylcarbonyl, butylcarbonyl, isobutylcarbonyl, pentylcarbonyl and hexylcarbonyl. Acetyl and ethylcarbonyl are particularly preferred.

(C₁-C₆)-Acyloxy represents for the purposes of the invention a straight-chain or branched acyl radical having 1 to 6, preferably 1 to 4, carbon atoms which is bonded via an oxygen atom. Preferred examples which may be mentioned are: acetyloxy, ethylcarbonyloxy, propylcarbonyloxy, isopropylcarbonyloxy, butylcarbonyloxy, isobutylcarbonyloxy, pentylcarbonyloxy and hexylcarbonyloxy. Acetyloxy and ethylcarbonyloxy are particularly preferred.

(C₁-C₆)-Acylamino represents for the purposes of the invention a straight-chain or branched acyl radical having 1 to 6, preferably 1 to 4, carbon atoms which is bonded via a nitrogen atom. Preferred examples which may be mentioned are: acetylamino, ethylcarbonylamino, propylcarbonylamino, isopropylcarbonylamino, butylcarbonylamino, isobutylcarbonylamino, pentylcarbonylamino and hexylcarbonylamino. Acetylamino and ethylcarbonylamino are particularly preferred.

(C₁-C₆)-Alkanediyl represents for the purposes of the invention a straight-chain or branched alkanediyl radical having 1 to 6, preferably 1 to 4, carbon atoms. Preferred examples which may be mentioned are methylene, ethylene, ethane-1,1-diyl, propylene, propane-1,2-diyl, propane-2,2-diyl. Methylene is preferred.

(C₁-C₆)-Alkoxy represents a straight-chain or branched alkoxy radical having 1 to 6, preferably 1 to 4, carbon atoms. Preferred examples which may be mentioned are: methoxy, ethoxy, n-propoxy, isopropoxy, tert-butoxy, n-pentoxy and n-hexoxy.

(C₁-C₆)-Alkoxycarbonyl represents a straight-chain or branched alkoxycarbonyl radical having 1 to 6, preferably 1 to 4, carbon atoms. Preferred examples which may be mentioned are: methoxycarbonyl, ethoxycarbonyl, n-propoxycarbonyl, isopropoxycarbonyl and tert-butoxycarbonyl.

(C₁-C₁₀)-, (C₁-C₆)- and (C₁-C₃)-Alkyl represent a straight-chain or branched alkyl radical having, respectively, 1 to 10, 1 to 6 and 1 to 3 carbon atoms. Preference is given in the case of (C₁-C₁₀)-alkyl to a straight-chain or branched alkyl radical having 1 to 6 carbon atoms, in the case of (C₁-C₆)-alkyl to a straight-chain or branched alkyl radical having 1 to 4 carbon atoms, and in the case of (C₁-C₃)-alkyl to methyl. Preferred examples which may be mentioned are: methyl, ethyl, n-propyl, isopropyl, tert-butyl, n-pentyl and n-hexyl. A straight-chain or branched alkyl radical having 1 to 3 carbon atoms is particularly preferred.

(C₁-C₆)-Alkylthio represents for the purposes of the invention a straight-chain or branched alkylthio radical having 1 to 6, preferably 1 to 4, carbon atoms. Preferred examples which may be mentioned are: methylthio, ethylthio, n-propylthio, isopropylthio, tert-butylthio, n-pentylthio and n-hexylthio. A straight-chain or branched alkylthio radical having 1 to 3 carbon atoms is particularly preferred.

Mono-(C₁-C₆)-alkylamino represents for the purposes of the invention an amino group having a straight-chain or branched alkyl substituent which has 1 to 6, preferably 1 to 4, carbon atoms. Preferred examples which may be mentioned are: methylamino, ethylamino, n-propylamino, isopropylamino, cyclopropylamino, t-butylamino, n-pentylamino, cyclopentylamino and n-hexylamino.

Di-(C₁-C₆)-alkylamino represents for the purposes of the invention an amino group having two identically or different straight-chain or branched alkyl substituents each of which has 1 to 6, preferably 1 to 4, carbon atoms. Preferred examples which may be mentioned are: N,N-dimethylamino, N,N-diethylamino, N-ethyl-N-methylamino, N-methyl-N-n-propylamino, N-methyl-N-cyclopropylamino, N-isopropyl-N-n-propylamino, N-t-butyl-N-methylamino, N-ethyl-N-n-pentylamino and N-n-hexyl-N-methylamino.

Mono-(C₁-C₆)-alkylaminocarbonyl represents for the purposes of the invention an amino group having a straight-chain or branched alkyl substituent which has 1 to 6, preferably 1 to 4, carbon atoms and is bonded via a carbonyl group. Preferred examples which may be mentioned are: methylaminocarbonyl, ethylaminocarbonyl, n-propylaminocarbonyl, isopropylaminocarbonyl, cyclopropylaminocarbonyl, t-butylaminocarbonyl, n-pentylaminocarbonyl, cyclopentylaminocarbonyl and n-hexylaminocarbonyl.

Di-(C₁-C₆)-alkylaminocarbonyl represents for the purposes of the invention an amino group having two identical or different straight-chain or branched alkyl substituents each of which has 1 to 6, preferably 1 to 4, carbon atoms and which is bonded via a carbonyl group. Preferred examples which may be mentioned are: N,N-dimethylaminocarbonyl, N,N-diethylaminocarbonyl, N-ethyl-N-methylaminocarbonyl, N-methyl-N-n-propylaminocarbonyl, N-methyl-N-cyclopropylaminocarbonyl, N-isopropyl-N-n-propylaminocarbonyl, N-t-butyl-N-methylaminocarbonyl, N-ethyl-N-n-pentylaminocarbonyl and N-n-hexyl-N-methylaminocarbonyl.

(C₁-C₈)-Cycloalkyl represents for the purposes of the invention a cycloalkyl group having 3 to 8, preferably 5 to 7, carbon atoms. Preferred examples which may be mentioned are: cyclopropyl, cyclobutyl, cyclopentyl, cyclohexyl and cycloheptyl.

(C₆-C₁₀)-Aryl represents for the purposes of the invention an aromatic radical having 6 to 10 carbon atoms. Preferred aryl radicals are phenyl and naphthyl.

Halogen represents for the purposes of the invention generally fluorine, chlorine, bromine and iodine. Fluorine, chlorine and bromine are preferred. Fluorine and chlorine are particularly preferred.

3- to 12-membered carbocyclyl represents for the purposes of the invention generally a mono- or polycyclic, carbocyclic radical having 3 to 12 ring atoms. 3- to 10-membered, in particular 3- to 8-membered, carbocyclyl are preferred. Mono- or bicyclic carbocyclyl is preferred. Monocyclic carbocyclyl is particularly preferred. The carbocyclyl radicals may be saturated or partially unsaturated. Saturated carbocyclyl radicals are preferred. Likewise preferred are (C₃-C₁₀)-cycloalkyl, very particularly (C₄-C₇)-cycloalkyl. Preferred examples which may be mentioned are: cyclopropyl, cyclobutyl, cyclopentyl, cyclopentenyl, cyclohexyl, cycloheptyl, norborn-1-yl, norborn-2-yl, norborn-7-yl, norborn-2-en-7-yl, cyclooctyl, cubyl, cyclononyl, cyclodecyl, decalinyl, adamant-1-yl, adamant-2-yl.

5- to 10-membered heteroaryl represents for the purposes of the invention generally an aromatic, mono- or bicyclic radical having 5 to 10 ring atoms and up to 5 heteroatoms from the series S, O and/or N. 5- to 6-membered heteroaryl having up to 4 heteroatoms are preferred. The heteroaryl radical may be bonded via a carbon atom or heteroatom. Preferred examples which may be mentioned are: thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

4- to 12-membered and 5- to 7-membered heterocyclyl represent for the purposes of the invention generally a mono- or polycyclic, heterocyclic radical having, respectively, 4 to 12 and 5 to 7 ring atoms and up to 3, preferably 2, heteroatoms or hetero groups from the series N, O, S, SO, SO₂. 5- to 7-membered heterocyclyl is preferred. Mono- or bicyclic heterocyclyl is preferred. Monocyclic heterocyclyl is particularly preferred. O, N and S are preferred as heteroatoms. The heterocyclyl radicals may be saturated or partially unsaturated. Saturated heterocyclyl radicals are preferred. The heterocyclyl radicals may be bonded via a carbon atom or a heteroatom. 5- to 7-membered, monocyclic saturated heterocyclyl radicals having up to two heteroatoms from the series O, N and S are particularly preferred. Preferred examples which may be mentioned are: tetrahydrofuran-2-yl, pyrrolidin-2-yl, pyrrolidin-3-yl, pyrrolinyl, piperidinyl, morpholinyl, perhydroazepinyl.

If radicals in the compounds of the invention are optionally substituted, the radicals may, unless otherwise specified, be substituted one or more times identically or differently. Substitution by up to three identical or different substituents is preferred.

Preference is given to compounds of the general formulae (I) and (Ia)

in which

A, E, Y, R¹, R², R³, R⁴, R⁵, R⁶, R⁷ R⁸ and R⁹ have the abovementioned meaning,

and where R⁶ and R⁷ are not both hydrogen,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Particular preference is given to compounds of the general formulae (I) and (Ia)

in which

A is methylene, and

E, Y, R¹, R², R³, R⁴, R⁵, R⁶, R⁷, R⁸ and R⁹ have the abovementioned meaning,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Particular preference is likewise given to compounds of the general formulae (I) and (Ia),

in which

Y is CO and

E, A, R¹, R², R³, R⁴, R⁵, R⁶, R⁷, R⁸ and R⁹ have the abovementioned meaning,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Particular preference is likewise given to compounds of the general formulae (I) and (Ia)

in which

R⁵ is phenyl which is optionally substituted identically or differently by one to three radicals selected from the group of methyl, chlorine, trifluoromethyl, trifluoromethoxy,

E is a bond, and

A, Y, R¹, R², R³, R⁴, R⁶, R⁷, R⁸ and R⁹ have the abovementioned meaning,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Particular preference is likewise given to compounds of the general formulae (I) and (Ia)

in which

A is (C₁-C₆)-alkanediyl,

E is a bond or (C₁-C₆)-alkanediyl,

Y is CO,

R¹, R², R³ and R⁴ are identical or different and are hydrogen, halogen, trifluoromethyl, trifluoromethoxy, hydroxyl, nitro, cyano, amino, (C₁-C₆)-alkyl, (C₁-C₆)-alkoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl or carboxyl,

R⁵ is (C₆-C₁₀)-aryl or 5- to 10-membered heteroaryl, where aryl and heteroaryl are optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4dioxabutane-1,4-diyl,

in which phenoxy, phenyl and 5- to 6-membered heteroaryl are in turn optionally substituted identically or differently by trifluoromethyl, (C₁-C₆)-alkyl, (C₁-C₆)-alkoxy or halogen,

R⁶ and R⁷ are identical or different and are hydrogen, (C₆-C₁₀)-aryl, 5- to 10-membered heteroaryl, 3- to 12-membered carbocyclyl, 4- to 12-membered heterocyclyl, or

are (C₁-C₁₀)-alkyl which is optionally substituted by halogen or a radical selected from the group of (C₁-C₆)-alkoxy, (C₆-C₁₀)-aryl, 5- to 10-membered heteroaryl, 3- to 12-membered carbocyclyl and 4- to 12-membered heterocyclyl,

where aryl, heteroaryl, heterocyclyl and carbocyclyl are optionally substituted identically or differently by radicals selected from the group of halogen, trifluoromethyl, trifluoromethoxy, hydroxyl, nitro, cyano, amino, (C₁-C₆)-alkyl, (C₃-C₈)-cycloalkyl, 5- to 7-membered heterocyclyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,

or

R⁶ and R⁷ together with the nitrogen atom form a 4- to 12-membered heterocyclyl radical which is bonded via nitrogen and which is optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, mono- or di-(C₁-C₆)-alkylamino, (C₁-C₆)-acyloxy, (C₁-C₆)-acyl, (C₁-C₆)-acylamino, (C₁-C₆)-alkoxycarbonyl, mono- or di-(C₁-C₆)-alkylaminocarbonyl, carbamoyl, carboxyl, (C₃-C₈)-cycloalkyl and phenyl,

where alkyl, cycloalkyl and phenyl in turn are optionally substituted identically or differently by one to three radicals selected from the group of halogen, phenyl, (C₁-C₆)-alkyl, (C₁-C₆)-alkoxy and (C₁-C₆)-alkylthio, in which phenyl in turn is optionally substituted identically or differently by radicals selected from the group of halogen or methyl,

R⁸ is hydrogen,

R⁹ is hydrogen,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Very particular preference is given to compounds of the general formulae (I) and (Ia)

in which

A is methylene or ethylene,

E is a bond, methylene or ethylene,

Y is CO,

R¹, R², R³ and R⁴ are identical or different and are hydrogen, halogen, methyl, ethyl, methoxy, ethoxy, carbamoyl or carboxyl,

R⁵ is (C₆-C₁₀)-aryl or 5- to 10-membered heteroaryl, where aryl and heteroaryl are optionally substituted identically or differently by radicals selected from the group of halogen, trifluoromethyl, trifluoromethoxy, methyl, ethyl, isopropyl, methoxy, ethoxy, phenoxy, dimethylamino, (C₁-C₆)-alkoxycarbonyl, carbamoyl, carboxyl, phenyl, 5- to 6-membered heteroaryl and butane-1,4-diyl,

where phenoxy, phenyl and 5- to 6-membered heteroaryl are optionally substituted by trifluoromethyl, (C₁-C₆)-alkyl, (C₁-C₆)-alkoxy or halogen,

R⁶ and R⁷ are identical or different and and are hydrogen, phenyl, 3- to 12-membered carbocyclyl, 4- to 12-membered heterocyclyl, where R⁶ and R⁷ are not both hydrogen, or

are (C₁-C₁₀)-alkyl which is optionally substituted by a radical selected from the group of halogen, (C₁-C₆)-alkoxy, (C₆-C₁₀)-aryl, 5- to 10-membered heteroaryl, 3- to 12-membered carbocyclyl and 4- to 12-membered heterocyclyl,

where aryl, heteroaryl, heterocyclyl and carbocyclyl are optionally substituted identically or differently by radicals selected from the group of halogen, trifluoromethyl, trifluoromethoxy, (C₁-C₆)-alkyl, (C₃-C₈)-cycloalkyl, 5- to 7-membered heterocyclyl, (C₁-C₆)-alkoxy, phenoxy, (C₁-C₆)-alkoxycarbonyl, carbamoyl, carboxyl, phenyl, 5- to 6-membered heteroaryl and butane-1,4-diyl,

or

R⁶ and R⁷ together with the nitrogen atom form a 4- to 12-membered heterocyclyl radical which is bonded via nitrogen and which is optionally substituted identically or differently by one to three radicals selected from the group of halogen, trifluoromethyl, trifluoromethoxy, (C₁-C₆)-alkyl, (C₁-C₆)-alkoxy, (C₁-C₆)-alkoxycarbonyl, carbamoyl, carboxyl, (C₃-C₈)-cycloalkyl and phenyl,

where alkyl, cycloalkyl and phenyl in turn are optionally substituted identically or differently by radicals selected from the group of halogen, phenyl, (C₁-C₆)-alkyl und (C₁-C₆)-alkoxy,

R⁸ is hydrogen or (C₁-C₃)-alkyl which is optionally substituted by fluorine,

R⁹ is hydrogen or (C₁-C₆)-alkyl,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Very particular preference is likewise given to compounds of the general formulae (I) and (Ia)

in which

A is methylene or ethylene,

E is a bond, methylene or ethylene,

Y is CO,

R¹, R², R³ and R⁴ are identical or different and are hydrogen, halogen, methyl, ethyl, methoxy, ethoxy, carbamoyl or carboxyl,

R⁵ is (C₆-C₁₀)-aryl or 5- to 10-membered heteroaryl, where aryl and heteroaryl are optionally substituted identically or differently by radicals selected from the group of halogen, trifluoromethyl, trifluoromethoxy, methyl, ethyl, isopropyl, methoxy, ethoxy, phenoxy, dimethylamino, (C₁-C₆)-alkoxycarbonyl, carbamoyl, carboxyl, phenyl, 5- to 6-membered heteroaryl and butane-1,4-diyl,

where phenoxy, phenyl and 5- to 6-membered heteroaryl are optionally substituted by trifluoromethyl, (C₁-C₆)-alkyl, (C₁-C₆)-alkoxy or halogen,

R⁶ and R⁷ are identical or different and are hydrogen, phenyl, 3- to 12-membered carbocyclyl, 4- to 12-membered heterocyclyl, where R⁶ and R⁷ are not both hydrogen, or

are (C₁-C₁₀)-alkyl which is optionally substituted by a radical selected from the group of halogen, (C₁-C₆)-alkoxy, (C₆-C₁₀)-aryl, 5- to 10-membered heteroaryl, 3- to 12-membered carbocyclyl and 4- to 12-membered heterocyclyl,

where aryl, heteroaryl, heterocyclyl and carbocyclyl are optionally substituted identically or differently by radicals selected from the group of halogen, trifluoromethyl, trifluoromethoxy, (C₁-C₆)-alkyl, (C₃-C₈)-cycloalkyl, 5- to 7-membered heterocyclyl, (C₁-C₆)-alkoxy, phenoxy, (C₁-C₆)-alkoxycarbonyl, carbamoyl, carboxyl, phenyl, 5- to 6-membered heteroaryl and butane-1,4-diyl,

or

R⁶ and R⁷ together with the nitrogen atom form a 4- to 12-membered heterocyclyl radical which is bonded via nitrogen and which is optionally substituted identically or differently by one to three radicals selected from the group halogen, trifluoromethyl, trifluoromethoxy, (C₁-C₆)-alkyl, (C₁-C₆)-alkoxy, (C₁-C₆)-alkoxycarbonyl, carbamoyl, carboxyl, (C₃-C₈)-cycloalkyl and phenyl,

where alkyl, cycloalkyl and phenyl in turn are optionaly substituted identically or differently by radicals selected from the group of halogen, phenyl, (C₁-C₆)-alkyl and (C₁-C₆)-alkoxy,

R⁸ is hydrogen,

R⁹ is hydrogen,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Very particular preference is likewise given to compounds of the general formulae (I) and (Ia)

in which

A is methylene,

E is a bond,

Y is CO,

R¹, R² R³ and R⁴ are identical or different and each is hydrogen, methyl or halogen,

R⁵ is phenyl which is optionally substituted identically or differently by one to three radicals selected from the group of methyl, isopropyl, methoxy, ethoxy, halogen, p-chlorophenoxy, trifluoromethyl and trifluoromethoxy,

R⁶ and R⁷ are identical or different and

are hydrogen, phenyl or 5- to 8-membered carbocyclyl, where R⁶ and R⁷ are not both hydrogen,

or

are (C₁-C₆)-alkyl which is optionally substituted by a radical selected from the group of (C₁-C₆)-alkoxy, phenyl, 5- to 8-membered carbocyclyl and 5- to 8-membered heterocyclyl,

where phenyl, heterocyclyl and carbocyclyl are optionally substitituted identically or differently by one to three radicals selected from the group of halogen, trifluoromethyl, trifluoromethoxy, (C₁-C₆)-alkyl, (C₃-C₈)-cycloalkyl, 5- to 7-membered heterocyclyl, (C₁-C₆)-alkoxy, (C₁-C₆)-alkoxycarbonyl and butane-1,4-diyl, and

R⁸ is hydrogen,

R⁹ is hydrogen,

and the salts, hydrates and/or solvates thereof,

with the exception of 2-[3-oxo-1-(phenylsulfonyl)-1,2,3,4-tetrahydro-2-quinoxalinyl]-N-phenylacetamide.

Very particular preference is likewise given to compounds of the general formulae (I) and (Ia)

in which

A is methylene,

E is a bond,

Y is CO,

R¹, R², R³ and R⁴ are identical or different and are hydrogen or halogen,

R⁵ is phenyl which is optionally substituted identically or differently by one to three radicals selected from the group of methyl, isopropyl, halogen, trifluoromethyl and trifluoromethoxy,

R⁶ and R⁷ are identical or different and

are hydrogen, (C₁-C₆)-alkyl, phenyl or 5- to 8-membered carbocyclyl, where R⁶ and R⁷ are not both hydrogen, and where carbocyclyl and phenyl is optionally substituted identically or differently by radicals selected from the group of halogen, trifluoromethyl, trifluoromethoxy, methyl and methoxy,

R⁸ is hydrogen,

R⁹ is hydrogen,

and the salts, hydrates and/or solvates thereof,

with the exception of 2-[3-oxo-1-(phenylsulfonyl)-1,2,3,4-tetrahydro-2-quinoxalinyl]-N-phenylacetamide.

The invention further relates to processes for preparing the compounds of the formulae (I) and (Ia).

In process

[A] compounds of the general formula (II) or (IIa)

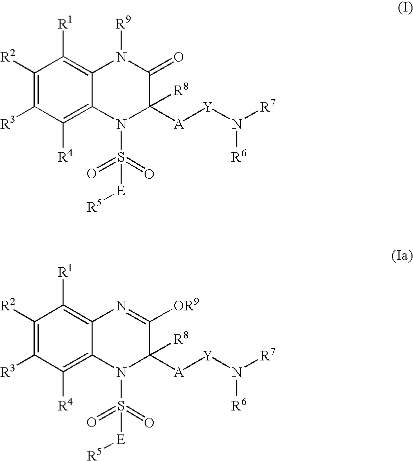

in which

A, E, Y, R¹, R², R³, R⁴, R⁵, R⁸ and R⁹ have the abovementioned meaning, and

X¹ is halogen, preferably bromine or chlorine, or hydroxyl,

are reacted with compounds of the general formula (III)

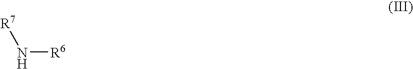

or the salts thereof, e.g. hydrochloride or hydrobromide salts,

in which

R⁶ and R⁷ have the abovementioned meaning

in the case where X¹ is halogen,

in inert solvents, where appropriate in the presence of a base, preferably in a temperature range from 0° C. to 50° C. under atmospheric pressure, to give compounds of the general formula (I) or (Ia).

Examples of inert solvents are halohydrocarbons such as methylene chloride, trichloromethane, tetrachloromethane, trichloroethane, tetrachloroethane, 1,2-dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2-dimethoxyethane, 2-butanone, dimethyl sulfoxide, acetonitrile, pyridine or hexamethylphosphoric triamide, with preference for tetrahydrofuran or methylene chloride.

Examples of bases are alkali metal hydroxides such as sodium or potassium hydroxide, or alkali metal carbonates such as cesium carbonate, sodium or potassium carbonate, or amides such as lithium diisopropylamide, or other bases such as DBU, triethylamine or diisopropylethylaamine, preferably triethylamine.

In the case where X¹ is hydroxyl,

compounds of the general formula (II) or (IIa)

are reacted in inert solvents in the presence of conventional condensing agents, where appropriate in the presence of a base, preferably in a temperature range from room temperature to 50° C. under atmospheric pressure, to give compounds of the general formula (I) or (Ia).

Examples of inert solvents are halohydrocarbons such as methylene chloride, trichloromethane, tetrachloromethane, trichloroethane, tetrachloroethane, 1,2 dichloroethane or trichloroethylene, ethers such as diethyl ether, methyl tert-butyl ether, dioxane, tetrahydrofuran, glycol dimethyl ether or diethylene glycol dimethyl ether, hydrocarbons such as benzene, xylene, toluene, hexane, cyclohexane or petroleum fractions, or other solvents such as nitromethane, ethyl acetate, acetone, dimethylformamide, dimethylacetamide, 1,2 dimethoxyethane, dimethyl sulfoxide, acetonitrile or pyridine, with preference for tetrahydrofuran, dimethylformamide or methylene chloride.

Examples of conventional condensing agents are carbodiimides such as, for example, N,N′-diethyl-, N,N,′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium-3-sulfate or 2-tert-butyl-5-methylisoxazolium perchlorate, or acylamino compounds such as 2-ethoxy-1-ethoxycarbonyl-1,2-dihydroquinoline, or propanephosphonic acid, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)-phosphonium hexafluorophosphate (BOP), or mixtures thereof.

Examples of bases are alkali metal carbonates such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

Particular preference is given to the combination of N-(3-dimethylaminoisopropyl)-N′-ethylcarbodiimide hydrochloride (EDC) and 1-hydroxybenzotriazole (HOBt), and to the combination of N-cyclohexylcarbodiimide-N′-propyloxymethyl-polystyrene (PS-carbodiimide) and 1-hydroxybenzotriazole (HOBt) and to the combination of O-(7-azabenzotriazol-1-yl)-N,N,N′,N′-tetramethyluronium hexafluorophosphate (HATU) and diisopropylethylamine.

The compounds of the general formula (III) are known or can be synthesized from the appropriate precursors by known processes.

The preparation of the compounds of the general formulae (II) and (IIa) is described hereinafter: (II-1) and (IIa-1) for Y═CO, (II-2) and (IIa-2) for Y═SO₂.

In process

[B] compounds of the general formula (Ib)

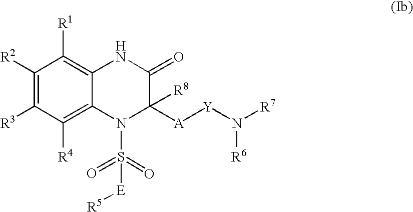

in which

A, E, Y, R¹, R², R³, R⁴, R⁵, R⁶, R⁷ and R⁸ have the abovementioned meaning, are reacted

with compounds of the general formula (IV),

X²—R¹⁰ (IV)

in which

R¹⁰ is (C₁-C₆)-alkyl, and

X² is halogen, preferably bromine or iodine,

in inert solvents in the presence of a base, where appropriate in the presence of potassium iodide, preferably in a temperature range from room temperature to the reflux of the solvent under atmospheric pressure, to give compounds of the general formula (I) or (Ia).

Examples of inert solvents are halohydrocarbons such as methylene chloride, trichloromethane or 1,2-dichloroethane, ethers such as dioxane, tetrahydrofuran or 1,2-dimethoxyethane, or other solvents such as acetone, dimethylformamide, dimethylacetamide, 2-butanone or acetonitrile, preferably tetrahydrofuran and methylene chloride.

Examples of bases are alkali metal carbonates such as cesium carbonate, sodium or potassium carbonate, or sodium or potassium methanolate, or sodium or potassium ethanolate or potassium tert-butoxide, or other bases such as sodium hydride, potassium hexadimethyldisilazide, lithium hexadimethyldisilazide or DBU, preferably potassium hexadimethyldisilazide or sodium hydride.

The compounds of the general formula (IV) are known or can be synthesized from the appropriate precursors by known processes.

In process

[C] compounds of the general formula (V)

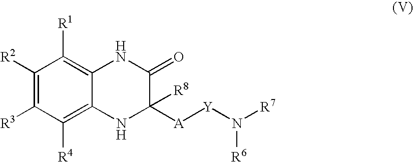

in which

A, Y, R¹, R², R³, R⁴, R⁶, R⁷ and R⁸ have the abovementioned meaning, are reacted

with compounds of the general formula (VI),

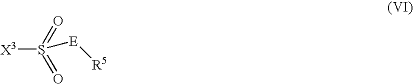

in which

E and R⁵ have the abovementioned meaning, and

X³ is halogen, preferably bromine or chlorine,

in inert solvents, where appropriate in the presence of a base, preferably in a temperature range from room temperature to the reflux of the solvent under atmospheric pressure, to give compounds of the general formula (Ib).

Examples of inert solvents are halohydrocarbons such as methylene chloride, trichloromethane or 1,2-dichloroethane, ethers such as diethyl ether, dioxane, tetrahydrofuran or 1,2-dimethoxyethane, hydrocarbons such as benzene, xylene or toluene, or other solvents such as acetone, dimethylformamide, 2-butanone, acetonitrile or pyridine, with preference for pyridine, acetonitrile, methylene chloride or tetrahydrofuran.

Examples of bases are alkali metal carbonates such as cesium carbonate, sodium or potassium carbonate, or sodium or potassium methanolate, or sodium or potassium triethylamine, diisopropylethylamine or pyridine, with preference for alkali metal carbonates such as cesium carbonate, sodium or potassium carbonate or pyridine.

The compounds of the general formula (VI) are known or can be synthesized from the appropriate precursors by known processes.

The compounds of the general formula (V) can be prepared from the appropriate precursors in analogy to synthetic processes indicated hereinafter for the compounds of the general formula (X).

Compounds of the general formula (Va)

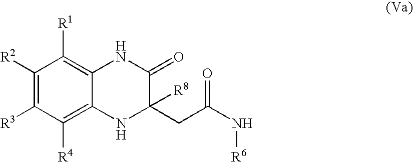

in which

R¹, R², R³, R⁴, R⁶ and R⁸ have the abovementioned meaning,

are prepared by reacting compounds of the general formula (VII)

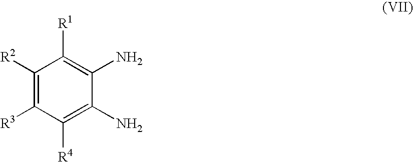

in which

R¹, R², R³ and R⁴ have the abovementioned meaning,

with compounds of the general formula (VIII)

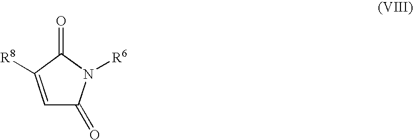

in which

R⁶ and R⁸ have the abovementioned meaning,

in inert solvents, preferably in a temperature range from room temperature to the reflux of the solvent under atmospheric pressure.

Examples of inert solvents are alcohols such as methanol, ethanol, n-propanol, isopropanol, n-butanol or tert-butanol, or mixtures of said solvents, where appropriate with water, with preference for a mixture of ethanol and water.

The compounds of the general formulae (VII) and (VIII) are known or can be synthesized from the appropriate precursors by known processes (cf. for (VIII): J. Romanenko, et al., Chem. Heterocycl. Compd. (Engl. Trans.) 9, 1973, 244).

Compounds of the general formula (II-1) or (IIa-1) are prepared by reacting compounds of the general formula (IX) or (IXa)

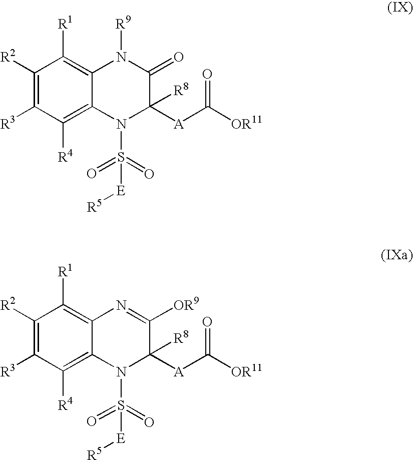

in which

A, E, R¹, R², R³, R⁴, R⁵, R⁸ and R⁹ have the abovementioned meaning, and

R¹¹ is (C₁-C₆)-alkyl, preferably methyl and ethyl,

with bases, in inert solvents, preferably in a temperature range from room temperature to 60° C. under atmospheric pressure.

Examples of bases are alkali metal hydroxides such as sodium, lithium or potassium hydroxide, or alkali metal carbonates such as cesium carbonate, sodium or potassium carbonate, preferably sodium hydroxide or lithium hydroxide.

Examples of inert solvents are halohydrocarbons such as methylene chloride, tetrachloromethane, trichloroethane, tetrachloroethane, 1,2-dichloroethane or trichloroethylene, ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, or mixtures of said solvents, where appropriate with water, preferably tetrahydrofuran and/or methanol or a mixture of water and ethanol or a mixture of water and dioxane.

Compounds of the general formula (IX) or (IXa) when R⁹═R¹⁰ are prepared by reacting compounds of the general formula (IXb)

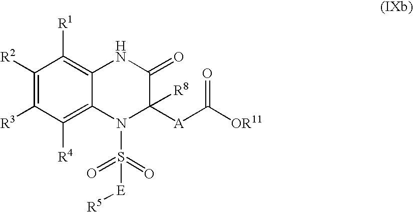

in which

A, E, R¹, R², R³, R⁴, R⁵, R⁸ and R¹¹ have the abovementioned meaning,

with compounds of the general formula (IV) under the reaction conditions described in process [B].

Compounds of the general formula (IXb) are prepared by reacting compounds of the general formula (X)

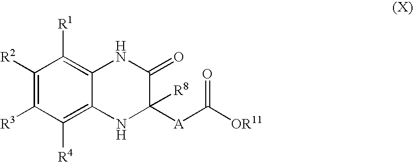

in which

A, R¹, R², R³, R⁴, R⁸ and R¹¹ have the abovementioned meaning,

with compounds of the general formula (VI) under the reaction conditions described in process [C].

Compounds of the general formula (X) are prepared by reacting compounds of the general formula (XI)

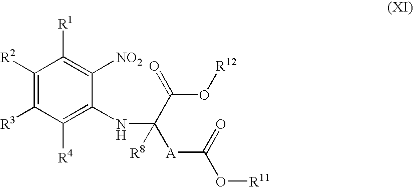

in which

A, R¹, R², R³, R⁴, R⁸ and R¹¹ have the abovementioned meaning, and

R¹² is (C₁-C₆)-alkyl, preferably methyl and ethyl,

under with a reducing agent in inert solvents, preferably in a temperature range from room temperature to the reflux of the solvent under atmospheric pressure up to 3 bar (cf. R. C. Larock, Comprehensive Organic Transformations, VCH Verlagsgesellschaft, 1989, pages 411-415).

Examples of reducing agents are palladium on activated carbon and hydrogen, tin dichloride or titanium trichloride, with preference for palladium on activated carbon and hydrogen or tin dichloride.

Examples of inert solvents are ethers such as diethyl ether, methyl tert-butyl ether, 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as ethyl acetate, dimethylformamide, dimethylacetamide, acetonitrile or pyridine, preferred solvents being methanol, ethanol, isopropanol or, in the case of tin dichloride, in ethanol, methanol or dimethylformamide.

Compounds of the general formula (XI) are prepared by reacting compounds of the general formula (XII)

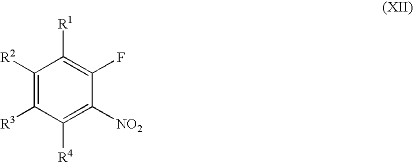

in which

R¹, R², R³ and R⁴ have the abovementioned meaning,

with compounds of the general formula (XIII),

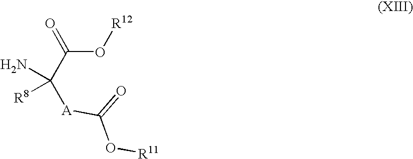

in which

A, R⁸, R¹¹ and R¹² have the abovementioned meaning,

or the salts thereof, e.g. hydrochloride or hydrobromide salts,

in inert solvents, where appropriate in the presence of a base, preferably in a temperature range from room temperature to the reflux of the solvent under atmospheric pressure.

Examples of inert solvents are ethers such as 1,2-dimethoxyethane, dioxane, glycol dimethyl ether or diethylene glycol dimethyl ether, hydrocarbons such as benzene, xylene, toluene or petroleum fractions, or other solvents such as dimethylformamide, dimethylacetamide, dimethyl sulfoxide, acetonitrile or pyridine, with dimethyl sulfoxide being preferred as solvent.

Examples of bases are alkali metal carbonates such as cesium carbonate, sodium or potassium carbonate, or amides such as sodamide, lithium bis(trimethylsilyl)amide, lithium diisopropylamide, or organometallic compounds such as butyllithium or phenyllithium, or other bases such as sodium hydride, DBU, triethylamine or diisopropylethylamine, preferably diisopropylethylamine or triethylamine.

The compounds of the general formulae (XII) and (XIII) are known or can be synthesized from the appropriate precursors by known processes [cf. for (VIII): Drysdale et al. Bioorg. Med. Chem. Lett. 1998, 8, 133-138.4; Aitken et al. Synthesis 1997, 787-791; Larsson et al. Acta Chem. Scand. 1994, 48, 517-525, Trost et al. J. Org. Chem. 1988, 53, 532).

Compounds of the general formula (II-2) or (IIa-2)

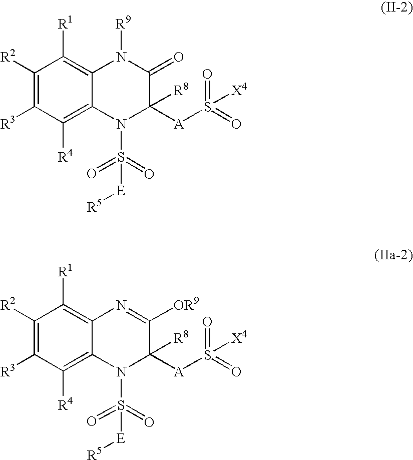

in which

A, E, R¹, R², R³, R⁴, R⁵, R⁸ and R⁹ have the abovementioned meaning, and

X⁴ is halogen, preferably chlorine,

are prepared by reacting compounds of the general formula (XIV) or (XIVa),

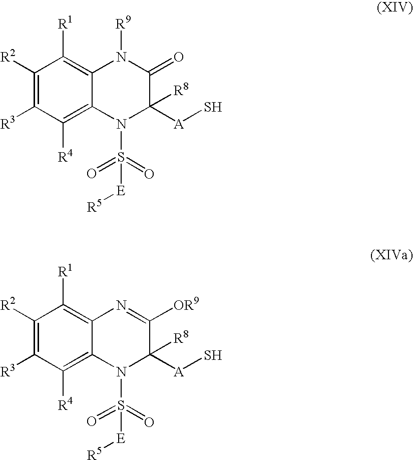

in which

A, E, R¹, R², R³, R⁴, R⁵, R⁸ and R⁹ have the abovementioned meaning,

with potassium nitrate and sulfuryl chloride in inert solvents, preferably in a temperature range from room temperature to the reflux of the solvent at atmospheric pressure.

Examples of inert solvents are halohydrocarbons such as methylene chloride, trichloromethane, tetrachloromethane, trichloroethane, tetrachloroethane, 1,2-dichloroethane or trichloroethylene, ethers such as 1,2-dimethoxyethane, dioxane, tetrahydrofuran, glycol dimethyl ether or diethylene glycol dimethyl ether, alcohols such as methanol, ethanol, n-propanol, isopropanol, n-butanol or tert-butanol, hydrocarbons such as benzene, xylene, toluene, hexane, cyclohexane or petroleum fractions, or other solvents such as ethyl acetate, dimethylformamide, dimethylacetamide, dimethyl sulfoxide, acetonitrile or pyridine, with acetonitrile being preferred as solvent.

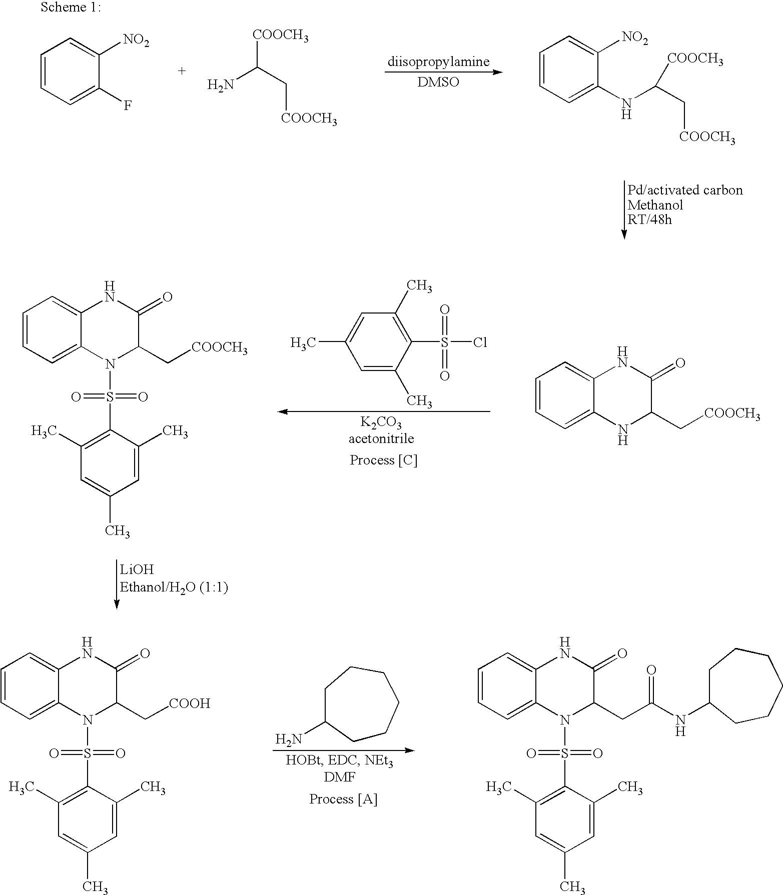

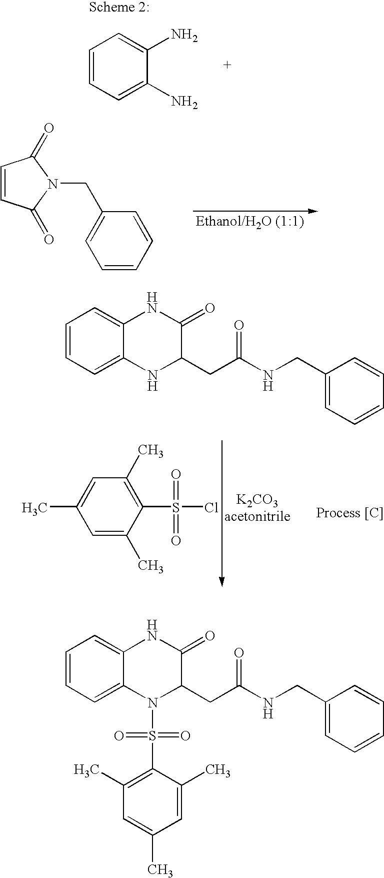

The compounds of the general formula (I) of the invention are suitable for use as medicaments for the treatment and/or prophylaxis of diseases in humans and animals.

The compounds of the invention show a valuable range of pharmacological effects which could not have been predicted.

The compounds of the invention have B1 receptor antagonistic effects.

The compounds of the invention can, by reason of their pharmacological properties, be employed alone or in combination with other medicaments for the prophylaxis and treatment of acute and/or chronic pain (for a classification, see “Classification of Chronic Pain, Descriptions of Chronic Pain Syndromes and Definitions of Pain Terms”, 2^{nd} Edition., Meskey and Begduk, Editors; IASP-Press, Seattle, 1994), especially for the treatment of cancer-induced pain and chronic neuropathic pain such as, for example, associated with diabetic neuropathy, post-herpetic neuralgia, peripheral nerve damage, central pain (for example resulting from cerebral ischemia) and trigeminal neuralgia, and other chronic pain such as, for example, lumbago, (low) back pain, inflammatory or rheumatic pain. These substances are moreover suitable for the therapy of primarily acute pain of any etiology and of secondary states of pain resulting therefrom, and for the therapy of states of pain which were formerly acute and have become chronic.

Bradykinin 1 antagonists are furthermore suitable for the treatment of asthma, diabetic vasculopathy, rhinitis, septic shock, atherosclerosis, multiple sclerosis or rheumatoid arthritis.

The in vitro effect of the compounds of the invention can be shown using the following biological assays:

1. Functional in vitro Assay

Agonists such as des-Arg9-BK and des-Arg10-kallidin activate the B1 receptor and lead, via stimulation of phospholipase C, to release of calcium ions from intracellular stores. Antagonists block the activation of the receptor by the agonists and thus also the agonist-dependent stimulation of phospholipase C and the intracellular calcium release induced thereby.

A functional in vitro assay can be carried out with stable cell lines, e.g. CHO or HEK 293, which recombinantly express the human B1 receptor. This entails measuring the activation of the receptor by the agonist indirectly via the intracellular calcium release induced thereby (in microtiter plates with 96, 384 and 1536 wells/plate). The effect of the tested substances can be stated as IC₅₀.

In this assay, Examples 170 and 177 have IC₅₀ values of 25 nM and 17 nM, respectively.

2. Binding to CHO BK1 Membranes

The binding of ligands to the B1 receptor from B1-transfected CHO cell membranes is carried out by the method of Levesque et al. (Immunopharmacol. 1995, 29, 141-147). Incubation buffer (Tris-HCl buffer pH 7.4+1 mM phenanthrolines, 0.14 g/l bacitracin), labeled radioligand [³H]-desArg10-kallidin (0.5 nM), DMSO or test stubstance all pipetted together, and then 250 μg of protein are added, and the mixture is thoroughly mixed and incubated at RT for 90 min. After expiry of the incubation time, the reaction is stopped by adding ice-cold Tris-HCl buffer to each tube. Filtration through Whatman GF/B filters (in 0.6% polyetylenimines) is followed by washing with 2×3 ml of Tris-HCl buffer. The filters are transferred into minivials, and the radioactivity is determined in a liquid scintillation counter. The effect of the tested substances can be stated as Kᵢ or IC₅₀.

The suitability of the compounds of the invention for the treatment of states of pain, especially neuropathic states of pain, can be shown in the following animal models:

3. Model of Acute Inflammatory Pain (Carrageenin Model) in Rats

This model follows the description by Winter et al. (Proc. Soc. Exp. Biol. Med., 1962, 111, 544-547).

Rats receive subplantar injections of a suspension of carrageenin in the right rear paw (0.35 mg per paw in 0.10 ml of physiological saline). Two hours later, the rats are thermally stimulated successively on the noninflamed and on the inflamed rear paw.

The thermal stimulation apparatus (Ugo Basile, Ref.: 7371) consists of 6 individual Plexiglas boxes (17×11×13 cm) placed on an elevated glass plate. A rat is is put in the box for 30 min for habituation. Then a movable infrared source (Setting 20) is focussed under the noninflamed and the inflamed rear paw, and the latency times until the paw is withdrawn are recorded automatically. The withdrawal of the paw interrupts the reflected beam and thus automatically switches off the counter and light source. To avoid tissue damage, the test is stopped after 45 s even if no paw-withdrawal response is recorded.

At least 12 rats are investigated in each group: male Wistar (Han) rats, 180-220 g. The test is carried out blind.

The data are analyzed by comparing the treated groups with the corresponding control by means of the unpaired Student's test.

The novel active ingredients can be converted in a known manner into conventional formulations such as tablets, coated tablets, pills, granules, aerosols, syrups, emulsions, suspensions and solutions, using inert, nontoxic, pharmaceutically suitable carriers or solvents. In these, the therapeutically active compound should be present in each case in a concentration of about 0.5 to 90% by weight of the complete mixture, i.e. in amounts which are sufficient to achieve the stated dosage range.

The formulations are produced for example by extending active ingredients with solvents and/or carriers, where appropriate with use of emulsifiers and/or dispersants, it being possible, for example when water is used as diluent, where appropriate to use organic solvents as auxiliary solvents.

Administration takes place in a conventional way, preferably orally, transdermally or parenterally, especially perlingually or intravenously. However, it can also take place by inhalation through the mouth or nose, for example with the aid of a spray, or topically via the skin.

It has generally proved advantageous to administer amounts of about 0.001 to 25 mg/kg, preferably about 0.1 to 10 mg/kg, of body weight, on oral use about 0.01 to 25 mg/kg, preferably about 0.5 to 5 mg/kg, of body weight to achieve effective results.

It may nevertheless be necessary where appropriate to deviate from the amounts mentioned, in particular as a function of the body weight and the mode of administration, of the individual response to the medicament, of the nature of its formulation and the time or interval over which administration takes place. Thus, in some cases it may sufficient to make do with less than the aforementioned minimum amount, whereas in other cases the upper limit mentioned must be exceeded. Where larger amounts are administered, it may be advisable to distribute these in a plurality of single doses over the day.

Abbreviations:

abs. absolute

Ac acetyl

acac acetylacetonyl

AIBN α, α′-azobis(isobutyronitrile)

Aloc allyloxycarbonyl

aq. aqueous

9-BBN 9-borabicyclo[3.3.1]nonane

Bn benzyl

Boc tert-butoxycarbonyl

Bom benzyloxymethyl

BOP benzotriazol-1-yloxytris(dimethylamino)phosphonium hexafluorophosphate

b.p. boiling point

Bu butyl

Bz benzoyl

CAN cerium ammonium nitrate

Cbz benzyloxycarbonyl

CDI N,N′-carbonyldiimidazole

cf. compare

CH cyclohexane

conc. concentrated

Cp cyclopentadienyl

cryst. crystalline/crystallized

CSA 10-camphorsulfonic acid

Dabco 1,4-diazabicyclo[2.2.2]octane

DAST diethylaminosulfur trifluoride

DBN 1,5-diazabicyclo[4.3.0]non-5-ene

DBU 1,8-diazabicyclo[5.4.0]undec-7-ene

DCC N,N′-dicyclohexylcarbodiimide

DCE 1,2-dichloroethane

DCI direct chemical ionization (in MS)

DCM dichloromethane

DDQ 2,3-dichloro-5,6-dicyano-1,4-benzoquinone

DEAD diethyl azodicarboxylate

d.e. diastereomeric excess

decomp. decomposition

DHP 3,4-dihydro-2h-pyran

DIAD diisopropyl azodicarboxylate

DIBAH diisobutylaluminum hydride

DIC diisopropylcarbodiimide

DIEA N,N-diisopropylethylamine

dil. dilute

dist. distilled

DMA N,N-dimethylacetamide

DMAP 4-N,N-dimethylaminopyridine

DME 1,2-dimethoxyethane

DMF N,N-dimethylformamide

DMPU N,N′-dimethylpropyleneurea

DMSO dimethyl sulfoxide

DNPH 2,4-dinitrophenylhydrazine

DPPA diphenylphosphoryl azide

EDC N′-(3-dimethylaminopropyl)-n-ethylcarbodiimide×HCl

e.e. enantiomeric excess

EA ethyl acetate (acetic acid ethyl ester)

EI electron impact ionization (in MS)

eq equivalent(s)

ESI electrospray ionization (in MS)

Et ethyl

Fmoc fluorenylmethoxycarbonyl

Fr. fraction

GC gas chromatography

HATU O-(7-azabenzotriazol-1-yl)-N,N,N′,N′-tetramethyluronium hexafluorophosphate

HBTU O-(benzotriazol-1-yl)-N,N,N′,N′-tetramethyluronium hexafluorophosphate

HMDS 1,1,1,3,3,3-hexamethyldisilazane

HMPA or HMPT hexamethylphosphoric triamide

HOBt 1-hydroxy-1H-benzotriazole×H₂O

HOSu N-hydroxysuccinimide

HPLC high pressure, high performance liquid chromatography

Im imidazol-1-yl

IR infrared spectroscopy

LAH lithium aluminum hydride

LC-MS coupled liquid chromatography-mass spectroscopy

LDA lithium N,N-diisopropylamide

LiHMDS lithium N,N-bistrimethylsilylamide

Lit. literature (reference)

Liq. liquid

m meta

mCPBA meta-chloroperbenzoic acid

Me methyl

MEK methyl ethyl ketone

MEM methoxyethoxymethyl

MOM methoxymethyl

m.p. melting point

MPLC medium pressure liquid chromatography

Ms methanesulfonyl (mesyl)

MS mass spectroscopy

MTBE methyl tert-butyl ether

MW molecular weight

NBS N-bromosuccinimide

NCS N-chlorosuccinimide

NIS N-iodosuccinimide

NMM N-methylmorpholine

NMO N-methylmorpholine N-oxide

NMR nuclear magnetic resonance spectroscopy

o ortho

p para

p.A. analytical grade

PCC pyridinium chlorochromate

PDC pyridinium dichromate

Pfp pentafluorophenyl

Ph phenyl

Piv pivaloyl

PMB p-methoxybenzyl

PNB p-nitrobenzyl

PPA polyphosphoric acid

ppt. precipitate

PPTS pyridinium p-toluenesulfonate

Pr propyl

PS polystyrene (resin)

py pyridine

PyBOP benzotriazol-1-yloxytris(pyrrolidino)phosphonium hexafluorophosphate

RF reflux

R_{f} retention index (in TLC)

RP reverse phase (in HPLC)

RT room temperature

Rₜ retention time (in HPLC)

sat. saturated

SEM 2-(trimethylsilyl)ethoxymethyl

sol. solution

subl. sublimes

TBAF tetrabutylammonium fluoride

TBAI tetrabutylammonium iodide

TBDMS tert-butyldimethylsilyl

TBDPS tert-butyldiphenylsilyl

TBTU O-(benzotriazol-1-yl)-N,N,N′,N′-tetramethyluronium tetrafluoroborate

TEA triethylamine

techn. technical

Teoc 2-(trimethylsilyl)ethoxycarbonyl

TES triethylsilyl

Tf trifluoromethanesulfonyl

TFA trifluoroacetic acid

TFAA trifluoroacetic anhydride

TfOH trifluoromethanesulfonic acid

THF tetrahydrofuran

THP tetrahydropyranyl

TIPS triisopropylsilyl

titr. titrated

TLC thin layer chromatography

TMEDA N,N,N′,N′-tetramethylethylenediamine

TMOF trimethyl orthoformate

TMS trimethylsilyl

TPP triphenylphosphine

TPPO triphenylphosphine oxide

Trt trityl

Ts p-toluenesulfonyl (tosyl)

TsOH p-toluenesulfonic acid

v/v volume-to-volume ratio (of a solution)

Vol. volume

w/w weight-to-weight ratio (of a solution)

Z benzyloxycarbonyl

The LC-MS data were found by the following methods:

Method A

HPLC apparatus type: HP 1100

UV dectector DAD: 208-400 nm

Column: symmetry C 18; 50 mm×2.1 mm; 3.5 μm

Ionization: ESI positive/negative

Oven temperature: 40° C.

Solvent A: CH₃CN+0.1% formic acid Solvent B: H₂O+0.1% formic acid

Gradient:

| Time | A:% | B:% | Flow rate |
|---|---|---|---|
| 0.00 | 10.0 | 90.0 | 0.50 |
| 4.00 | 90.0 | 10.0 | 0.50 |
| 6.00 | 90.0 | 10.0 | 0.50 |
| 6.10 | 10.0 | 90.0 | 1.00 |
| 7.50 | 10.0 | 90.0 | 0.50 |

Method B

Column: symmetry C 18; 2.1 mm×150 mm; 5 μm

Ionization: ESI positive/negative

Oven temperature: 70° C.

Solvent B: 0.3 g of HCl (30%)/1 l of water

Gradient: A/B 2/98 to 95/5 within 2.5 min

Flow rate: 0.9 ml/min to 1.2 ml/min within 2 min

Method C

Instrument: HP 1100 with DAD detection;

Column: Kromasil RP-18, 60 mm×2 mm, 3.5 μm;

Eluent: A=5 ml HClO4/l H2O, B=ACN;

Gradient: 0 min 2% B, 0.5 min 2% B, 4.5 min 90% B, 6.5 min 90% B;

Flow rate: 0.75 ml/min; Temp.: 30° C.; Detection UV 210 nm

Starting Compounds

### EXAMPLE I

### N-Phenylmethyl-2-(3-oxo-1,2,3,4-tetrahydro-2-quinoxalinyl)acetamide

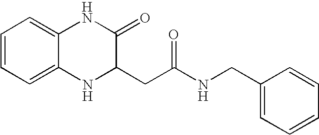

A suspension of 5.00 g (46.2 mmol) of 1,2-phenylenediamine and 8.66 g (46.2 mmol) of N-(phenylmethyl)maleimide in 500 ml of 1:1 ethanol/water are heated to boiling. After refluxing for 4 hours, the mixture is allowed to cool and the resulting precipitate is removed. The filter cake is washed with 1:1 ethanol/water and dried in vacuo. 6.64 g (49%) of the target compound are obtained in this way as a yellowish solid. The filtrate is evaporated and the residue is triturated in isopropanol. A further 1.37 g (10%) are obtained in this way as a pale yellow solid.

¹H-NMR (200 MHz, DMSO-d₆): δ=2.43 (dd, 1H), 2.71 (m, 1H), 4.10-4.16 (m, 1H), 4.16-4.42 (m, 2H), 5.90 (s, br, 1H), 6.54-6.85 (m, 4H), 7.17-7.39 (m, 5H), 8.45 (t, 1H), 10.28 (s, br, 1H).

MS (ESI): m/z=296 [M+H]⁺.

### EXAMPLE II

### N-(2-Methoxyphenyl)-2-(3-oxo-1,2,3,4-tetrahydro-2-quinoxalinyl)acetamide

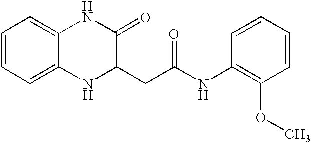

This compound is obtained in analogy to the method of example I from 1.00 g (9.25 mmol) of 1,2-phenylenediamine and 1.88 g (9.25 mmol) of N-(2-methoxyphenyl)maleimide after refluxing for 3.5 hours and triturating the resulting precipitate with isopropanol.

Yield: 1.76 g (61%) as pale yellow solid.

¹H-NMR (200 MHz, DMSO-d₆): δ=2.52-2.98 (ABX system, AB part, 2H), 3.74 (s, 3H), 4.21 (dd, 1H), 6.04 (s, 1H), 6.55-7.10 (m, 7H), 8.06 (d, 1H), 9.43 (s, 1H), 10.33 (s, 1H).

MS (DCI, NH₃): m/z=329 [M+NH₄]⁺, 312 [M+H]⁺.

### EXAMPLE III

### Dimethyl N-(2-nitrophenyl)aspartate

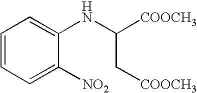

A solution of 53.6 g (380 mmol) of 1-fluoro-2-nitrobenzene, 25.0 g (127 mmol) of dimethyl DL-asparatate and 49.1 g (380 mmol) of N,N-diisopropylethylamine in 150 ml of DMSO is stirred in an argon atmosphere at 60° C. overnight. It is cooled to room temperature, and 300 ml each of water and ethyl acetate are added to the mixture. The aqueous phase is extracted three times with 300 ml of ethyl acetate each time, and the combined organic phases are washed twice with 100 ml of water each time. The organic phase is dried over sodium sulfate, and the solvent is distilled off in a rotary evaporator. The crude product is purified on a flash column (mobile phase: toluene). 21.8 g (61%) of the target compound are obtained.

HPLC: Kromasil C18 60×2 mm; Eluent: water+5‰ HClO₄/acetonitrile, T=30° C., Flow rate=0.75 ml/min, Rₜ=4.3 min.

MS (DCI, NH₃): m/z=300 [M+NH₄]⁺, 283 [M+H]⁺, 582.4 [2M+NH₄]⁺.

¹H-NMR (200 MHz, CDCl₃): δ=2.99 (d, 2H), 3.75 (s, 3H), 3.80 (s, 3H), 4.70 (m, 1H), 6.76 (dt, 1H), 6.85 (m, 1H), 7.48 (dt, 1H), 8.21 (dd, 1H), 8.52 (d, broad, 1H).

### EXAMPLE IV

### Methyl(3-oxo-1,2,3,4-tetrahydro-2-quinoxalinyl)acetate

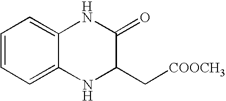

A solution of 22.0 g (77.9 mmol) of dimethyl N-(2-nitrophenyl)aspartate (example III) and catalytic amounts of palladium on activated carbon (10%) in 200 ml of methanol is stirred in a hydrogen atmosphere at room temperature for 48 h. Filtration with suction through kieselguhr is followed by washing with methanol. The solvent is distilled off in a rotary evaporator to result in 16.0 g (93%) of the desired product.

PLC: Kromasil C18 60×2 mm; Eluent: Water+5% HClO₄/acetonitrile, T=30° C., Flow rate=0.75 ml/min, Rₜ=3.4 min.

MS (DCI, NH₃): m/z=238.2 [M+NH₄]⁺, 221.2 [M+H]⁺.

¹H-NMR (200 MHz, CDCl₃): δ=2.72 (dd, 1H), 3.14 (dd, 1H), 3.75 (s, 3H), 4.34 (dt, 1H), 4.73 (s, broad, 1H), 6.79-6.82 (m, 3H), 6.91 (m, 1H), 8.21 (s, broad, 1H).

### EXAMPLE V

### Methyl 2-[1-(mesitylsulfonyl)-3-oxo-1,2,3,4-tetrahydro-2-quinoxalinyl]acetate

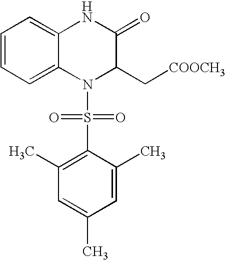

A solution of 4.00 g (18.2 mmol) of methyl 2-(3-oxo-1,2,3,4-tetrahydro-2-quinoxalinyl)acetate (example IV), 19.9 g (90.8 mmol) of 1,3,5-trimethylbenzenesulfonyl chloride and 12.5 g (90.8 mmol) of potassium carbonate in 100 ml of acetonitrile is stirred at 60° C. for 16 h. The solvent is distilled off in a rotary evaporator, and the residue is taken up in 50 ml of water and extracted three times with 100 ml of ethyl acetate each time. The combined organic phases are dried over sodium sulfate, and the solvent is distilled off in a rotary evaporator. The crude product is purified on a flash column (mobile phase: 9:1 toluene/ethyl acetate) to result in 4.55 g (62%) of the title compound.

HPLC: Kromasil C18 60×2 mm; Eluent: Water+5‰ HClO₄/acetonitrile, T=30° C., Flow rate=0.75 ml/min, Rₜ=4.4 min.

MS (DCI, NH3): m/z=420.1 [M+NH₄]⁺.

¹H-NMR (200 MHz, CDCl₃): δ=2.28 (s, 3H), 2.37 (m, 1H), 2.50 (s, 6H), 2.55 (m, 1H), 3.60 (s, 3H), 4.94 (dd, 1H), 6.82 (dd, 1H), 6.93 (s, 2H), 7.04 (m, 1H), 7.21 (m, 1H), 7.38 (m, 1H), 8.25 (s, broad, 1H).

### EXAMPLE VI

### 2-[1-(Mesitylsulfonyl)-3-oxo-1,2,3,4-tetrahydro-2-quinoxalinyl]acetic acid

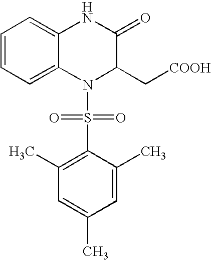

803 mg (33.5 mmol) of lithium hydroxide are added to a solution of 4.50 g (11.1 mmol) of methyl 2-[1-(mesitylsulfonyl)-3-oxo-1,2,3,4-tetrahydro-2-quinoxalinyl]acetate (example V) in 160 ml of water/ethanol (1:1) solution, and the mixture is stirred at RT for 4 h. Most of the ethanol is distilled off in a rotary evaporator, and the residue is mixed with 100 ml of ethyl acetate and adjusted to pH 2 with 1 molar aqueous hydrochloric acid. The aqueous phase is extracted six times with 100 ml of ethyl acetate each time. The combined organic phases are dried over sodium sulfate, and the solvent is distilled off in a rotary evaporator. 3.05 g (70%) of the title compound are obtained.

HPLC: Kromasil C18 60×2 mm; Eluent: Water+5‰ HClO₄/acetonitrile, T=30° C., Flow rate=0.75 ml/min, Rₜ=4.0 min.

MS (ESI): m/z=389.2 [M+H]⁺.

¹H-NMR (200 MHz, DMSO-d₆): δ=2.11 (dd, 1H), 2.27 (s, 3H), 2.38 (s, 6H), 2.41 (m, 1H), 4.68 (dd, 1H), 6.94 (dd, 1H), 7.00 (m, 1H), 7.05 (s, 2H), 7.07 (m, 1H), (dt, 1H).

### EXAMPLE VII

### 1-Cycloheptyl-1H-pyrrole-2,5-dione

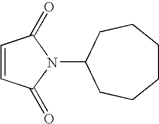

10 g (102 mmol) of maleic anhydride are dissolved in 600 ml of toluene at room temperature and then 11.54 g (102 mmol) of cycloheptylamine dissolved in 100 ml of toluene are slowly added. The reaction solution is then stirred at room temperature for one hour. 22.97 g (102 mmol) of zinc bromide are then added to the reaction solution heated to 80° C., and 32.27 ml (153 mmol) of hexamethyldisilazane (in 100 ml of toluene) are added dropwise over the course of 30 min. The solution is subsequently heated to 100° C. and stirred overnight. After the reaction solution has cooled, the solution is added to 200 ml of 0.5N HCl, and the organic phase is separated. The aqueous phase is extracted three times more with 200 ml of ethyl acetate, the combined organic phases are dried over magnesium sulfate, and the solvent is distilled off in a rotary evaporator. The crude product is purified on a flash column (mobile phase: cyclohexane/ethyl acetate). 18.78 g (94%) of the title compound are obtained as a white solid.

¹H-NMR (200 MHz, DMSO-d₆): δ=1.28-1.81 (m, 10H), 1.88-2.01 (m, 2H), 3.84-4.03 (m, 1H), 6.96 (s, 2H).

LC-MS: Rₜ=9.20;

MS (EI): m/z=193 [M⁺].

### EXAMPLE VIII

### N-Cycloheptyl-2-(oxo-1,2,3,4-tetrahydro-2-quinoxalinyl)acetic acid

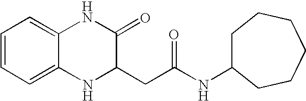

A suspension of 6.72 g (62.1 mmol) of 1,2-phenylenediamine and 12 g (62.1 mmol) of 1-cycloheptyl-1H-pyrrole-2,5-dione in 200 ml of 1:1 ethanol/water are heated to boiling. After refluxing for 12 hours, the mixture is allowed to cool, and the resulting precipitate is removed. The filter cake is washed with 1:1 ethanol/water and dried in vacuo.

16 g (85%) of the target compound are obtained in this way as a yellowish solid.

¹H-NMR (300 MHz, DMSO-d₆): δ=1.27-1.88 (m, 12H), 2.31 (dd, 1H), 2.59 (dd, 1H), 3.68-3.82 (m, 1H), 4.01-4.11 (m, 1H), 5.79 (s, br, 1H), 6.55-6.68 (m, 1H), 6.69-6.78 (m, 3H), 7.82 (d, 1H), 10.22 (s, br, 1H).

MS (ESI): m/z=302 [M+H]⁺.

Exemplary Embodiments

### EXAMPLE 1

### N-Cycloheptyl-2-[1-(mesitylsulfonyl)-3-oxo-1,2,3,4-tetrahydro-2-quinoxalinyl]-acetamnide

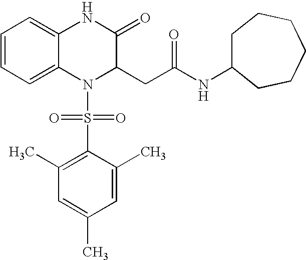

A solution of 1.00 g (2.57 mmol) of 2-[1-(mesitylsulfonyl)-3-oxo-1,2,3,4-tetrahydro-2-quinoxalinyl]acetic acid (example VI), 291 mg (2.57 mmol) of cycloheptylamine, 383 mg (2.83 mmol) of 1-hydroxy-1H-benzotriazole, 567 mg (2.96 mmol) of EDC and 521 mg (5.15 mmol) of triethylamine in 10 ml of DMF is stirred at room temperature overnight. The mixture is taken up in 100 ml of ethyl acetate and washed three times with 30 ml of water each time. The organic phase is washed twice with saturated sodium chloride solution and dried over sodium sulfate, and the solvent is distilled off in a rotary evaporator. The crude product is purified on a flash column (mobile phase: 100:2 dichloromethane/methanol). 550 mg (44%) of the title compound are obtained.

HPLC: Kromasil C18 60×2 mm; Eluent: Water+5‰ HClO₄/acetonitrile, T=30° C., Flow rate=0.75 ml/min, Rₜ=4.7 min.

MS (DCI, NH3): m/z=484.2 [M+H]⁺.

¹H-NMR (300 MHz, CDCl₃): δ=1.34-1.69 (m, 10H), 1.89 (m, 2H), 2.25 (s, 3H), 2.32 (dd, 1H), 2.41 (s, 6H), 2.50 (dd, 1H), 3.89 (m, 1H), 4.95 (dd, 1H), 5.92 (d, broad, 1H), 6.78 (dd, 1H), 6.90 (s, 2H), 7.05 (dt, 1H), 7.22 (dt, 1H), 7.41 (d, 1H), 7.59 (s, broad, 1H).

General Method for Preparing 2-[1-(mesitylsulfonyl)-3-oxo-1,2,3,4-tetrahydro-2-quinoxalinyl]acetamides Starting from Aliphatic Amines and the Compound of Example VI:

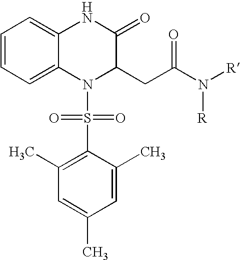

A solution of 0.07 mmol of the aliphatic primary or secondary amine, 40.4 mg (0.10 mmol) of 2-[1-(mesitylsulfonyl)-3-oxo-1,2,3,4-tetrahydro-2-quinoxalinyl]acetic acid (example VI), 15.8 mg (0.121 mmol) of 1-hydroxy-1H-benzotriazole and 200 mg of PS-carbodiimide (loading 0.94 mmol/g; from Argonaut) in 3 ml of dichloromethane is shaken at room temperature overnight. 200 mg of PS-trisamine (loading 3.85 mmol/g; from Argonaut) are added, and the mixture is shaken at room temperature for 6 hours. It is filtered through a silica gel cartridge (3 g) and washed with a 95:5 dichloromethane/methanol mixture. The mother liquor is mixed with 0.5 ml of a sat. sodium bicarbonate solution and shaken at RT for 30 min. It is filtered through an Extrelut/silica gel cartridge (1 g of each), and the solvent is distilled off in vacuo. The respective products are obtained and analyzed by LC/MS (Eluent:solution A=acetonitrile, solution B=water+0.6 g of HCl (30%)/l of water, Gradient: A/B 10:90 to A/B 90:10 within 4 min; Flow rate 0.6 ml/min; T=50° C.; Column: Kromasil RP-18, 2.1×150 mm).

Examples 2-106 listed in table 1 and 2 are obtained in accordance with this method. The optically active compounds in table 2 are prepared starting from dimethyl L-aspartate:

TABLE 1

| Ex. | | | | LC/MS |
|---|---|---|---|---|
| No. | Structure | Yield | LC-MS | method |
| 2 | 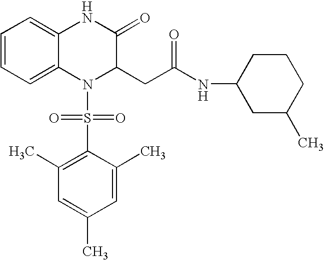 | 99% | Rt = 4.48 min; MS (ESIpos): 484 [M+H]+ | A |
| 3 | 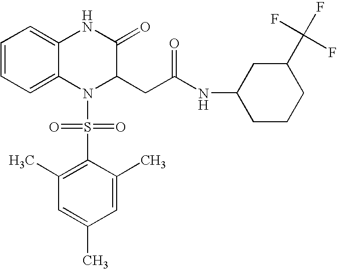 | 70% | Rt = 4.49 min; MS (ESIpos): 538 [M+H]+ | A |
| 4 | 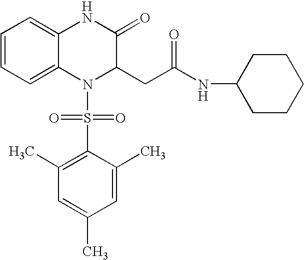 | 99% | Rt = 4.25 min; MS (ESIpos): 470 [M+H]+ | A |
| 5 | 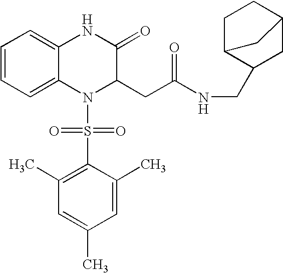 | 76% | Rt = 4.53 min; MS (ESIpos): 496 [M+H]+ | A |
| 6 | 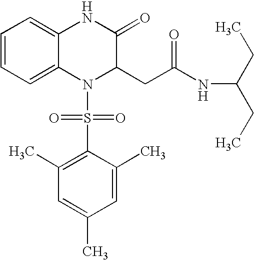 | 75% | Rt = 4.21 min; MS (ESIpos): 458 [M+H]+ | A |
| 7 | 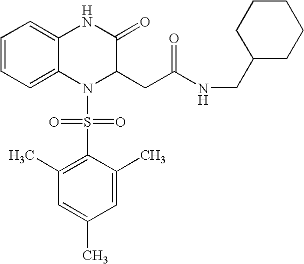 | 65% | Rt = 4.49 min; MS (ESIpos): 484 [M+H]+ | A |
| 8 | 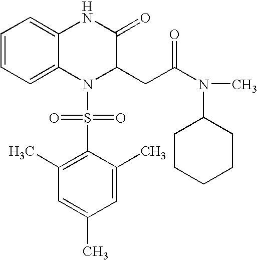 | 99% | Rt = 4.48 min; MS (ESIpos): 484 [M+H]+ | A |
| 9 | 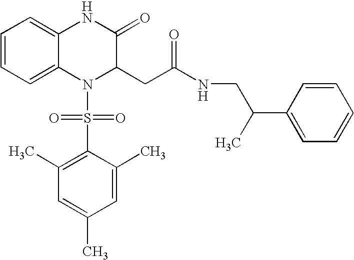 | 88% | Rt = 4.37 min; MS (ESIpos): 506 [M+H]+ | A |
| 10 | 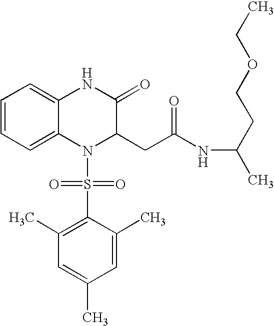 | 86% | Rt = 4.37 min; MS (ESIpos): 506 [M+H]+ | A |
| 11 | 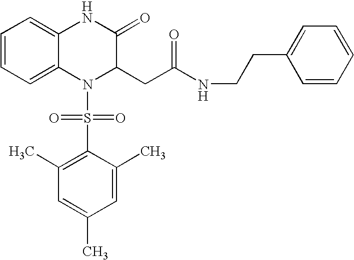 | 91% | Rt = 4.22 min; MS (ESIpos): 492 [M+H]+ | A |
| 12 | 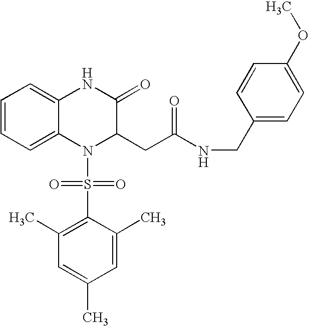 | 84% | Rt = 4.08 min; MS (ESIpos): 508 [M+H]+ | A |
| 13 | 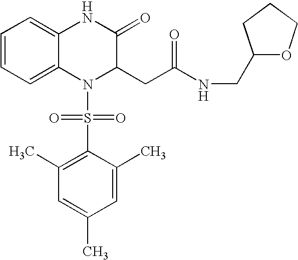 | 97% | Rt = 3.67 min; MS (ESIpos): 472 [M+H]+ | A |
| 14 | 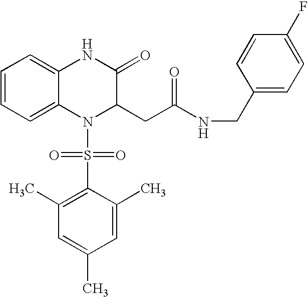 | 88% | Rt = 4.17 min; MS (ESIpos): 496 [M+H]+ | A |
| 15 | 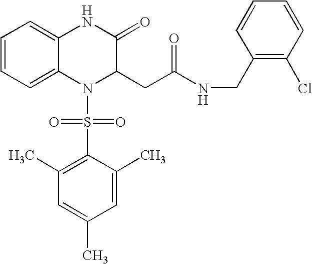 | 70% | Rt = 4.34 min; MS (ESIpos): 513 [M+H]+ | A |
| 16 | 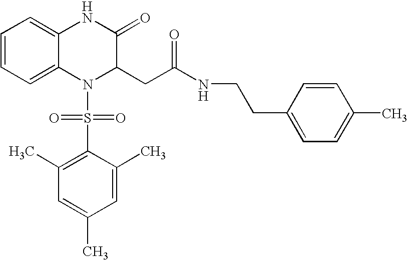 | 87% | Rt = 4.41 min; MS (ESIpos): 506 [M+H]+ | A |
| 17 | 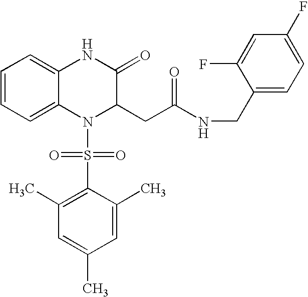 | 94% | Rt = 4.25 min; MS (ESIpos): 514 [M+H]+ | A |
| 18 | 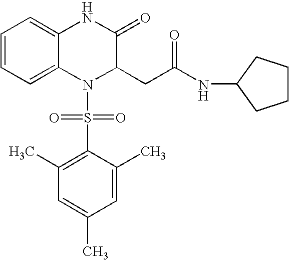 | 88% | Rt = 4.07 min; MS (ESIpos): 456 [M+H]+ | A |
| 19 | 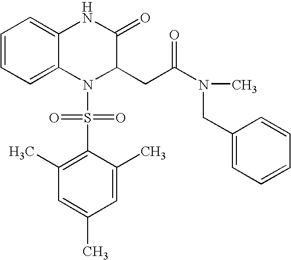 | 97% | Rt = 4.34 min; MS (ESIpos): 492 [M+H]+ | A |
| 20 | 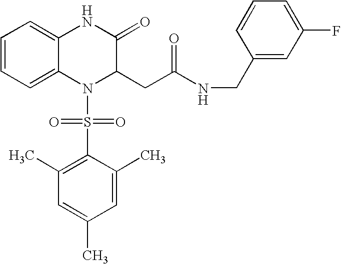 | 85% | Rt = 4.18 min; MS (ESIpos): 496 [M+H]+ | A |
| 21 | 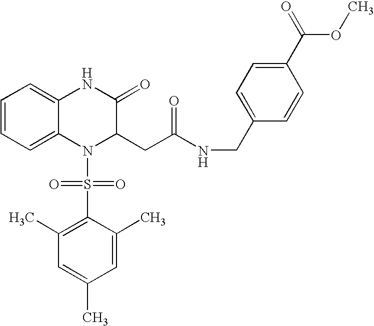 | 88% | Rt = 4.03 min; MS (ESIpos): 536 [M+H]+ | A |
| 22 | 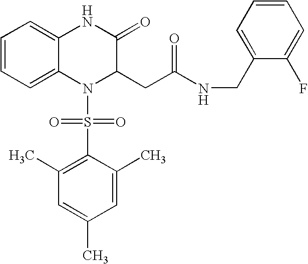 | 99% | Rt = 4.17 min; MS (ESIpos): 496 [M+H]+ | A |
| 23 | 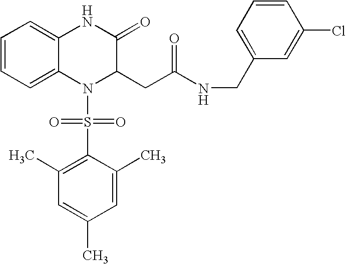 | 99% | Rt = 4.35 min; MS (ESIpos): 513 [M+H]+ | A |
| 24 | 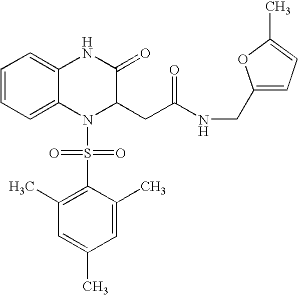 | 99% | Rt = 4.09 min; MS (ESIpos): 482 [M+H]+ | A |
| 25 | 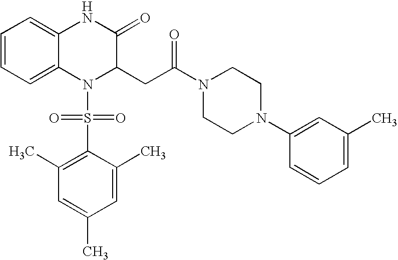 | 96% | Rt = 4.57 min; MS (ESIpos): 547 [M+H]+ | A |
| 26 | 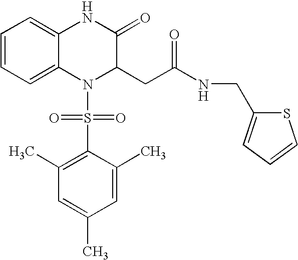 | 91% | Rt = 4.06 min; MS (ESIpos): 484 [M+H]+ | A |
| 27 | 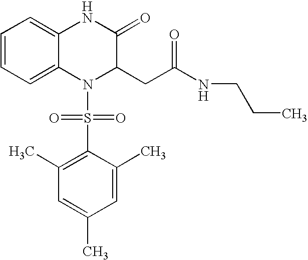 | 59% | Rt = 3.84 min; MS (ESIpos): 430 [M+H]+ | A |
| 28 | 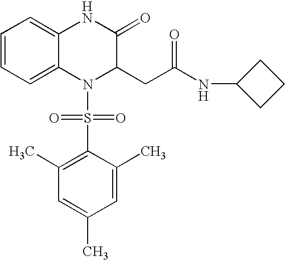 | 84% | Rt = 3.93 min; MS (ESIpos): 442 [M+H]+ | A |
| 29 | 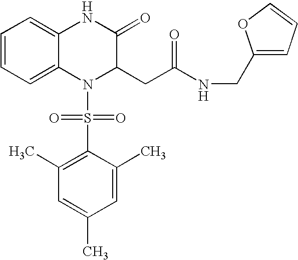 | 76% | Rt = 3.93 min; MS (ESIpos): 468 [M+H]+ | A |
| 30 | 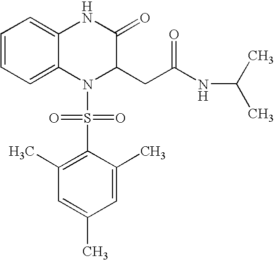 | 71% | Rt = 3.82 min; MS (ESIpos): 430 [M+H]+ | A |
| 31 | 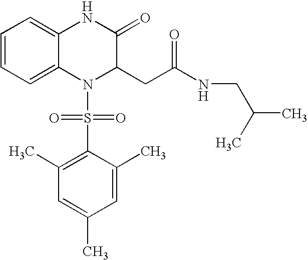 | 99% | Rt = 4.05 min; MS (ESIpos): 444 [M+H]+ | A |
| 32 | 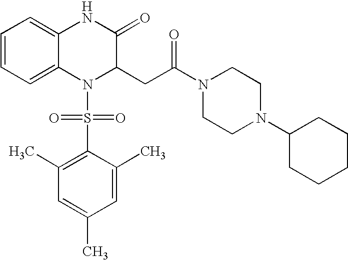 | 66% | Rt = 2.91 min; MS (ESIpos): 539 [M+H]+ | A |
| 33 | 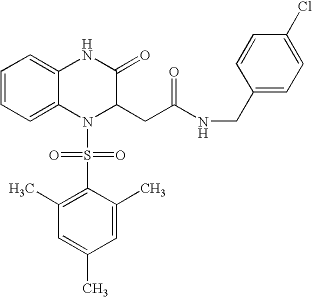 | 85% | Rt = 4.37 min; MS (ESIpos): 513 [M+H]+ | A |
| 34 | 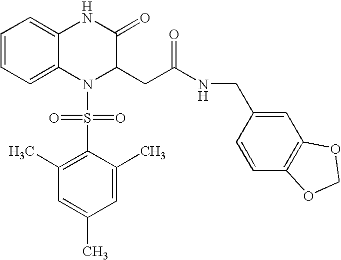 | 99% | Rt = 4.03 min; MS (ESIpos): 522 [M+H]+ | A |
| 35 | 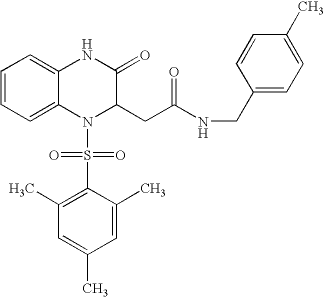 | 91% | Rt = 4.31 min; MS (ESIpos): 492 [M+H]+ | A |
| 36 | 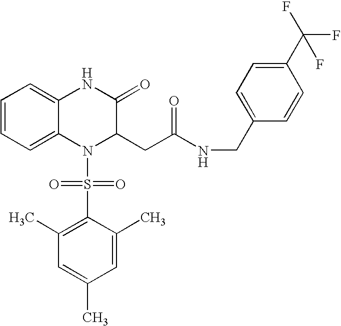 | 85% | Rt = 4.48 min; MS (ESIpos): 546 [M+H]+ | A |
| 37 | 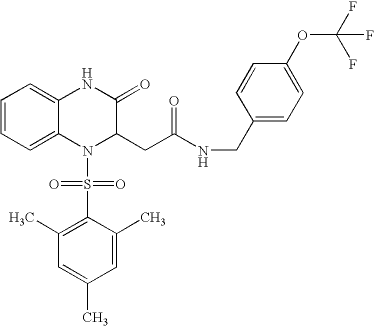 | 99% | Rt = 4.55 min; MS (ESIpos): 562 [M+H]+ | A |
| 38 | 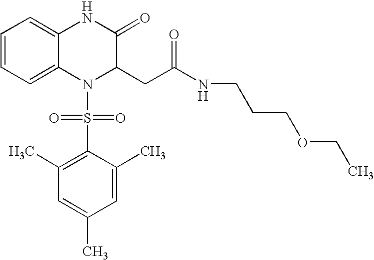 | 84% | Rt = 3.79 min; MS (ESIpos): 474 [M+H]+ | A |
| 39 | 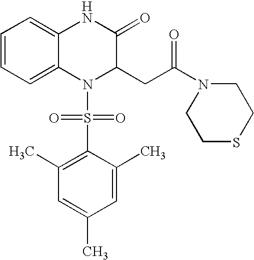 | 99% | Rt = 4.00 min; MS (ESIpos): 474 [M+H]+ | A |
| 40 | 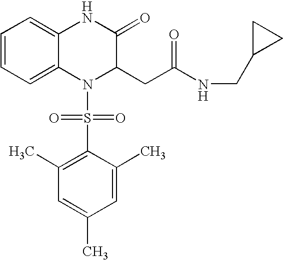 | 86% | Rt = 3.89 min; MS (ESIpos): 442 [M+H]+ | A |
| 41 | 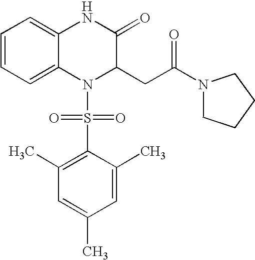 | 99% | Rt = 3.76 min; MS (ESIpos): 442 [M+H]+ | A |
| 42 | 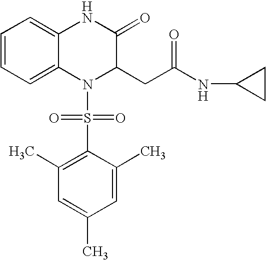 | 85% | Rt = 3.66 min; MS (ESIpos): 428 [M+H]+ | A |
| 43 | 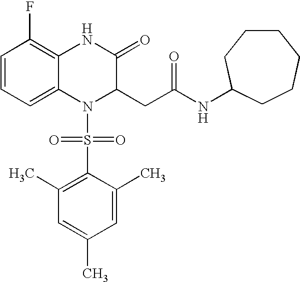 | 74% | Rt = 4.51 min; MS (ESIpos): 502 [M+H]+ | A |
| 44 | 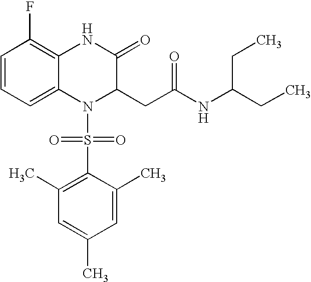 | 73% | Rt = 4.26 min; MS (ESIpos): 476 [M+H]+ | A |
| 45 | 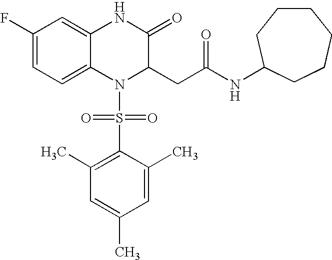 | 86% | Rt = 2.82 min; MS (ESIpos): 502 [M+H]+ | B |
| 46 | 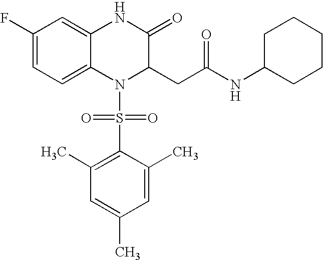 | 40% | Rt = 2.72 min; MS (ESIpos): 488 [M+H]+ | B |
| 47 | 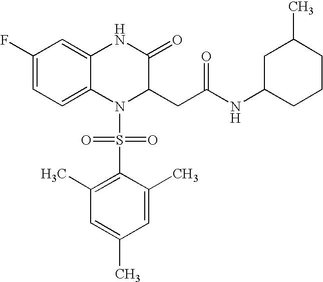 | 92% | Rt = 2.83 min; MS (ESIpos): 502 [M+H]+ | B |
| 48 | 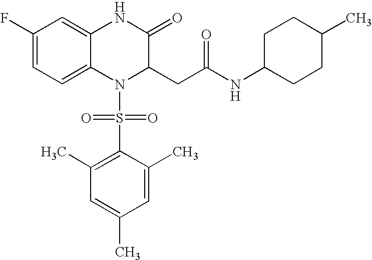 | 88% | Rt = 2.78 min; MS (ESIpos): 502 [M+H]+ | B |
| 49 | 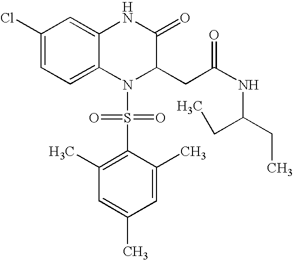 | 85% | Rt = 2.79 min; MS (ESIpos): 492 [M+H]+ | B |
| 50 | 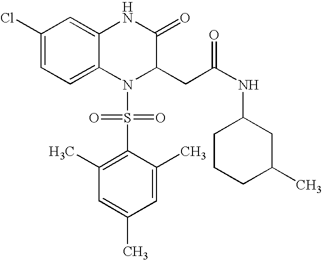 | 86% | Rt = 2.87 min; MS (ESIpos): 518 [M+H]+ | B |
| 51 | 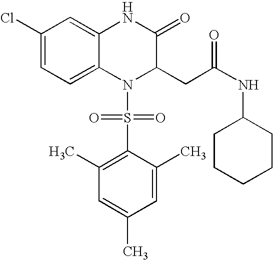 | 85% | Rt = 2.83 min; MS (ESIpos): 504 [M+H]+ | B |
| 52 | 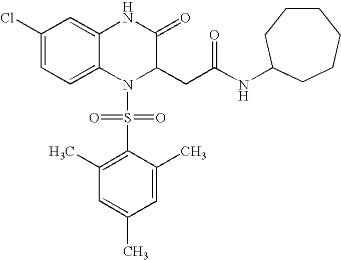 | 90% | Rt = 2.88 min; MS (ESIpos): 518 [M+H]+ | B |

TABLE 2

| | | MS | Rt | | LC/MS |
|---|---|---|---|---|---|
| Ex. No. | Structure | [M+H] | [min] | Yield | method |
| 53 | 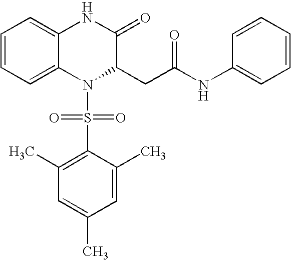 | 464 | 4.6 | 75% | A |
| 54 | 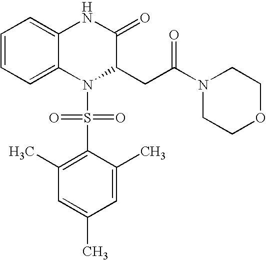 | 458 | 4 | 82% | A |
| 55 | 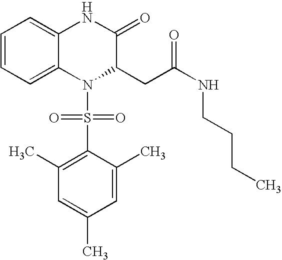 | 444 | 4.4 | quantitative | A |
| 56 | 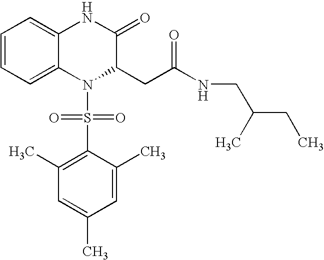 | 459 | 4.16 | quantitative | A |
| 57 | 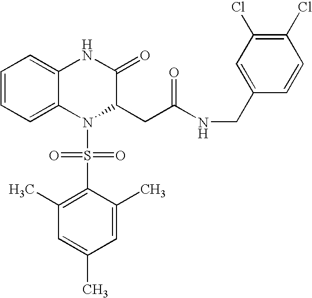 | 546 | 4.46 | 92% | A |
| 58 | 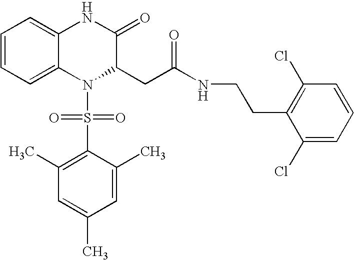 | 560 | 4.49 | 93% | A |
| 59 | 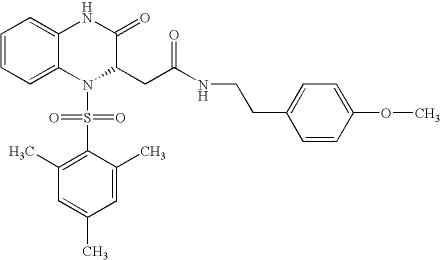 | 522 | 4.07 | quantitative | A |
| 60 | 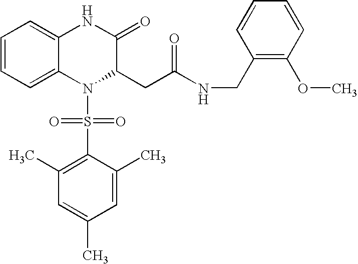 | 508 | 4.09 | quantitative | A |
| 61 | 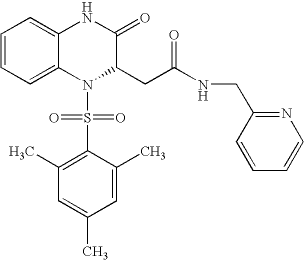 | 479 | 2.97 | quantitative | A |
| 62 | 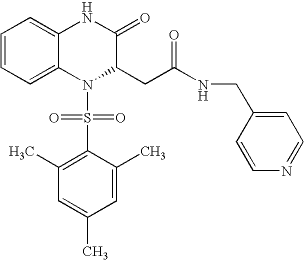 | 479 | 2.66 | 84% | A |
| 63 | 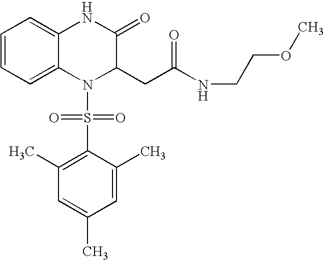 | 446 | 3.47 | quantitative | A |
| 64 | 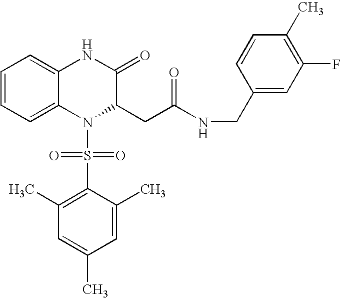 | 510 | 4.23 | 82% | A |
| 65 | 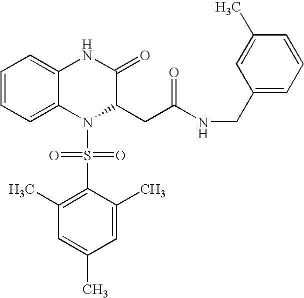 | 492 | 4.21 | quantitative | A |
| 66 | 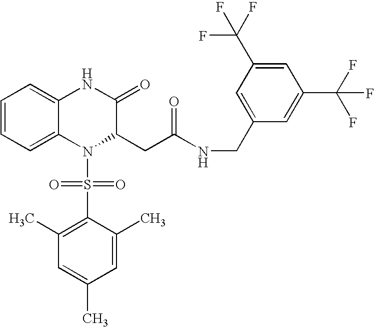 | 614 | 4.68 | quantitative | A |
| 67 | 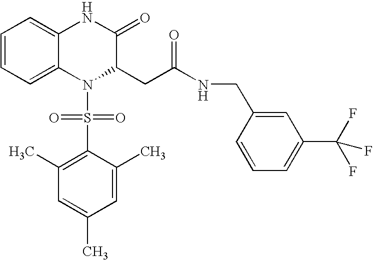 | 546 | 4.36 | quantitative | A |
| 68 | 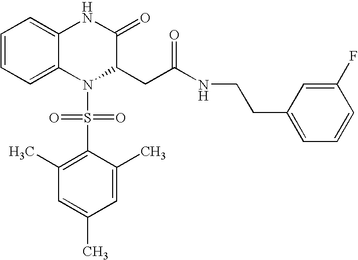 | 510 | 4.17 | 89% | A |
| 69 | 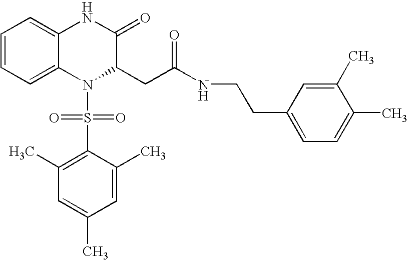 | 520 | 4.47 | quantitative | A |
| 70 | 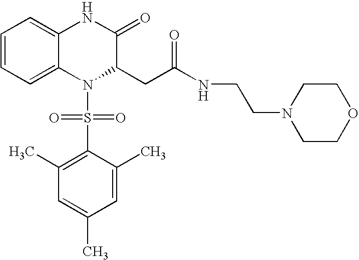 | 501 | 2.67 | quantitative | A |
| 71 | 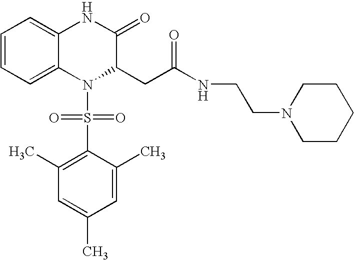 | 499 | 2.76 | 64% | A |
| 72 | 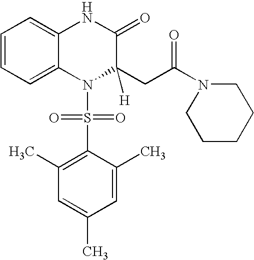 | 456 | 3.98 | 99% | A |
| 73 | 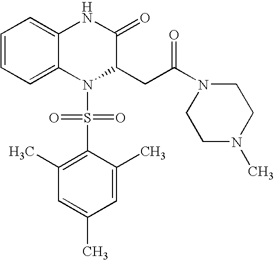 | 471 | 2.63 | 60% | A |
| 74 | 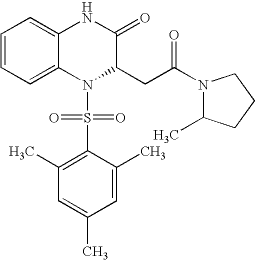 | 456 | 3.91 | 27% | A |
| 75 | 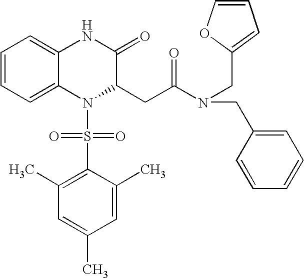 | 558 | 4.59 | 71% | A |
| 76 | 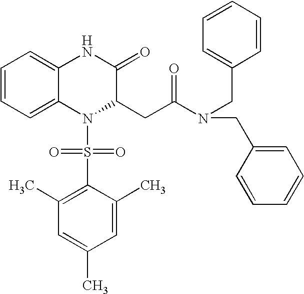 | 568 | 4.81 | 73% | A |
| 77 | 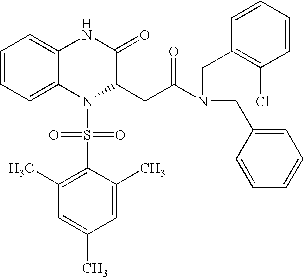 | 602 | 4.96 | 86% | A |
| 78 | 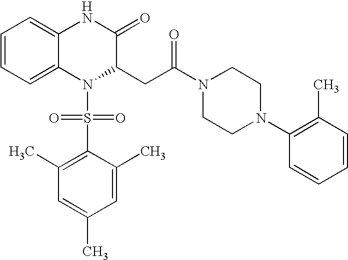 | 547 | 4.62 | 99% | A |
| 79 | 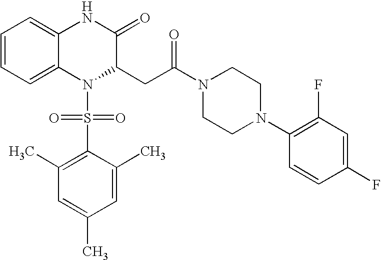 | 569 | 4.46 | 88% | A |
| 80 | 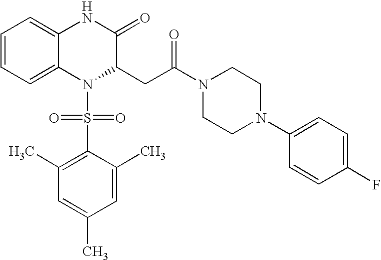 | 551 | 4.31 | 78% | A |
| 81 | 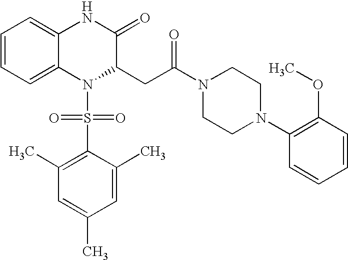 | 563 | 4.15 | 78% | A |
| 82 | 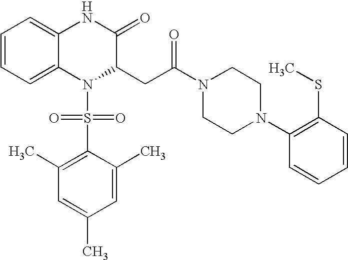 | 579 | 4.57 | 75% | A |
| 83 | 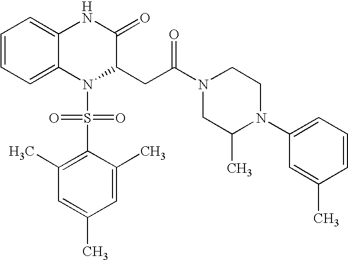 | 561 | 4.39 | 90% | A |
| 84 | 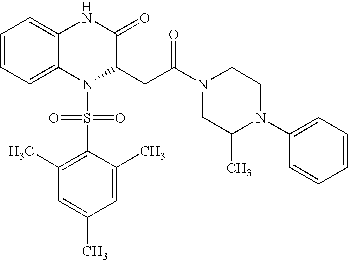 | 547 | 4.27 | 79% | A |
| 85 | 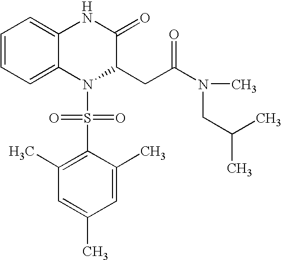 | 458 | 4.15 | 24% | A |
| 86 | 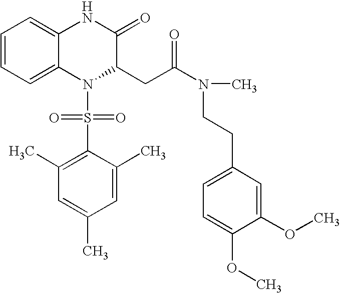 | 566 | 4.02 | 71% | A |
| 87 | 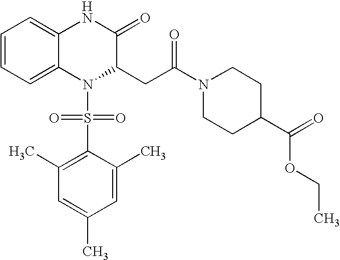 | 528 | 4 | 65% | A |
| 88 | 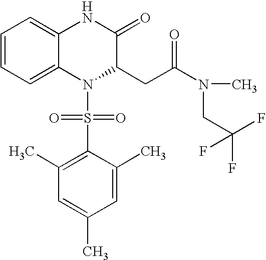 | 484 | 4.1 | 37% | A |
| 89 | 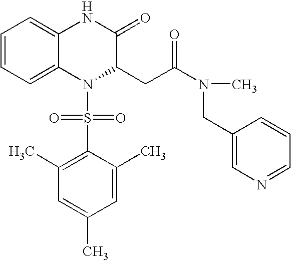 | 493 | 2.91 | 96% | A |
| 90 | 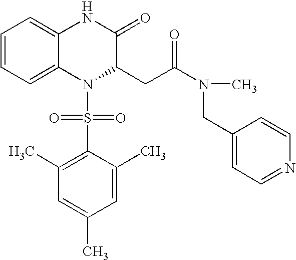 | 493 | 2.8 | 70% | A |
| 91 | 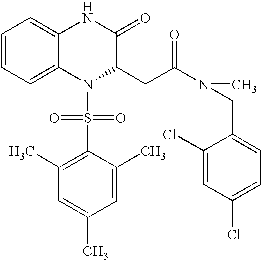 | 560 | 4.76 | 84% | A |
| 92 | 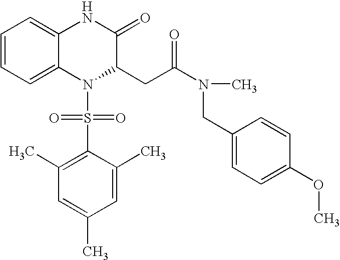 | 522 | 4.18 | 96% | A |
| 93 | 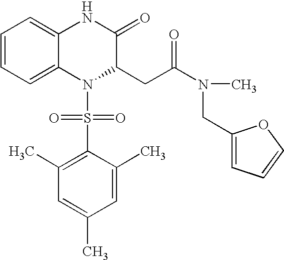 | 482 | 4.02 | 71% | A |
| 94 | 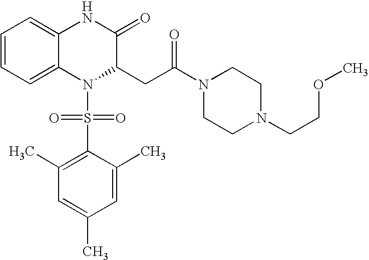 | 515 | 2.71 | 78% | A |
| 95 | 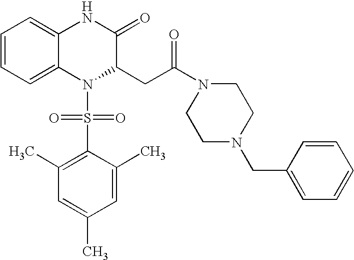 | 547 | 2.93 | 69% | A |
| 96 | 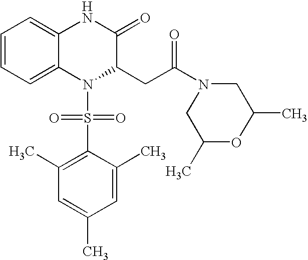 | 486 | 3.87 | 69% | A |
| 97 | 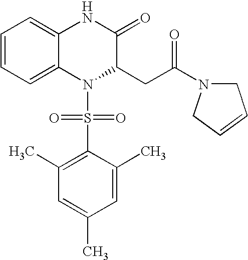 | 440 | 3.67 | 70% | A |
| 98 | 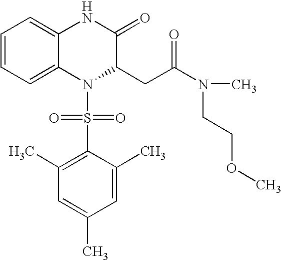 | 460 | 3.67 | 44% | A |
| 99 | 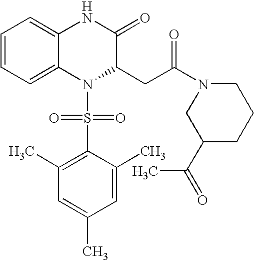 | 499 | 3.32 | 44% | A |
| 100 | 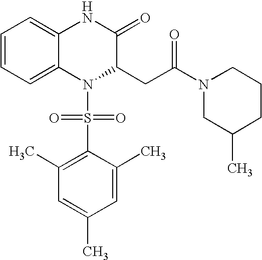 | 470 | 4.2 | 46% | A |
| 101 | 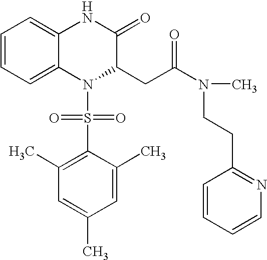 | 507 | 2.9 | 98% | A |
| 102 | 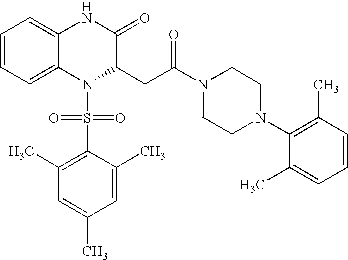 | 561 | 4.82 | 82% | A |
| 103 | 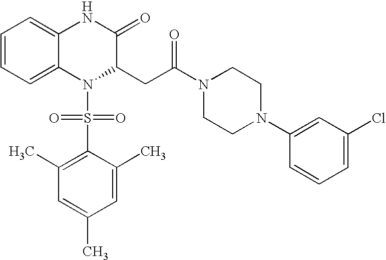 | 567 | 4.59 | 62% | A |
| 104 | 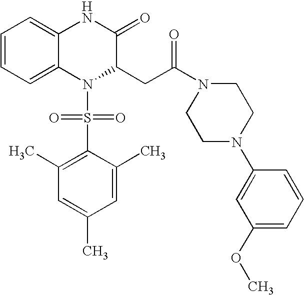 | 563 | 4.25 | 67% | A |
| 105 | 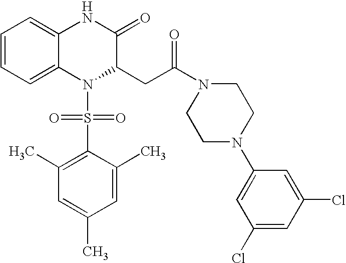 | 601 | 4.92 | 75% | A |
| 106 | 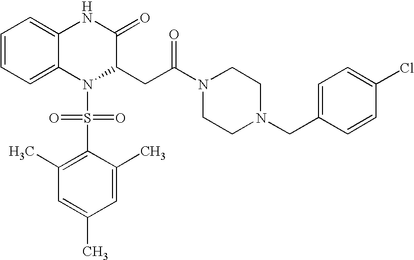 | 581 | 3.12 | 99% | A |

General Method for Preparing 2-[1-(mesitylsulfonyl)-3-oxo-1,2,3,4-tetrahydro-2-quinoxalinyl]acetamides Starting from Aromatic Amines and the Compound of Example VI:

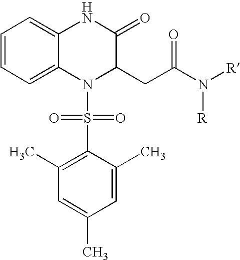

A solution of 0.08 mmol of the aromatic primary or secondary amine, 25.3 mg (0.07 mmol) of 2-[1-(mesitylsulfonyl)-3-oxo-1,2,3,4-tetrahydro-2-quinoxalinyl]acetic acid (example VI), 29.7 mg (0.08 mmol) of [O-(7-azabenzotriazol-1-yl)-1,1,3,3-tetramethyluronium]hexafluorophosphate (HATU) and 16.8 mg (0.13 mmol) of N,N-diisopropylethylamine in 3 ml of DMF is shaken at room temperature overnight. It is filtered and the solvent is distilled off. The residue is filtered through a silica gel cartridge and washed with a 90:10 dichloromethane/methanol solvent mixture. It is evaporated and the crude product is purified by a preparative RP-HPLC (Kromasil 100 C18, 5 μm; 50×20 mm; Gradient: acetonitrile/water 15:85 to 90:10, room temperature).

Examples 107-131 listed in table 3 are obtained in accordance with this method:

TABLE 3

| | | | | LC/MS |
|---|---|---|---|---|
| Example | Structure | Yield | LC-MS | method |
| 107 | 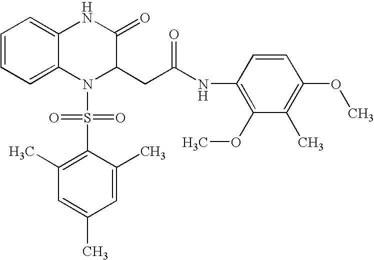 | 31% | Rt = 4.41 min; MS (ESIpos): 538 [M + H]+ | A |
| 108 | 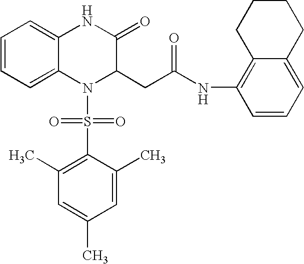 | 20% | Rt = 4.58 min; MS (ESIpos): 518 [M + H]+ | A |
| 109 | 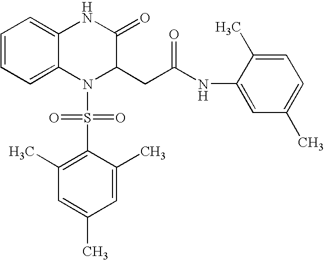 | 40% | Rt = 4.41 min; MS (ESIpos): 492 [M + H]+ | A |
| 110 | 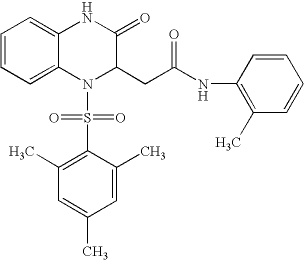 | 34% | Rt = 4.24 min; MS (ESIpos): 478 [M + H]+ | A |
| 111 | 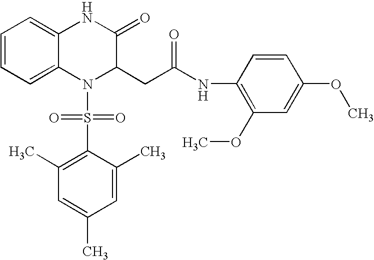 | 36% | Rt = 4.16 min; MS (ESIpos): 524 [M + H]+ | A |
| 112 | 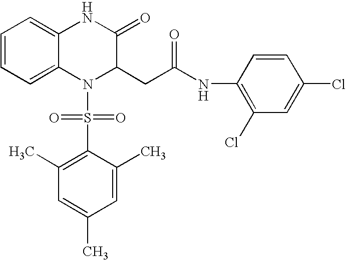 | 5% | Rt = 4.71 min; MS (ESIpos): 533 [M + H]+ | A |
| 113 | 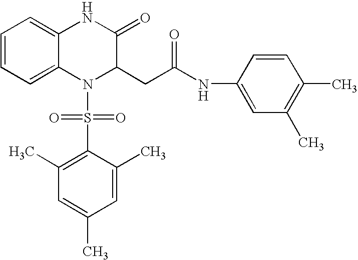 | 46% | Rt = 4.51 min; MS (ESIpos): 492 [M + H]+ | A |
| 114 | 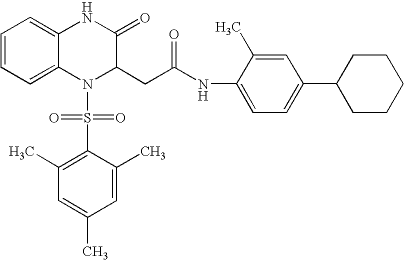 | 34% | Rt = 5.23 min; MS (ESIpos): 560 [M + H]+ | A |
| 115 | 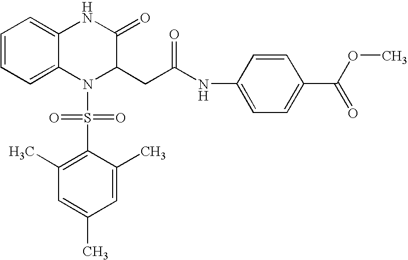 | 7% | Rt = 4.19 min; MS (ESIpos): 522 [M + H]+ | A |
| 116 | 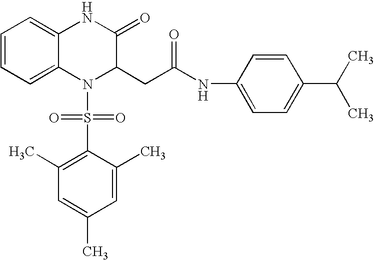 | 58% | Rt = 4.74 min; MS (ESIpos): 506 [M + H]+ | A |
| 117 | 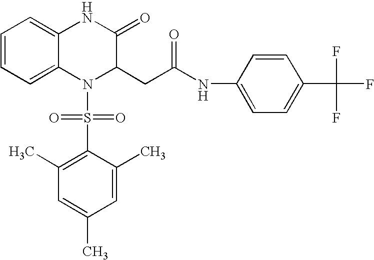 | 14% | Rt = 4.63 min; MS (ESIpos): 532 [M + H]+ | A |
| 118 | 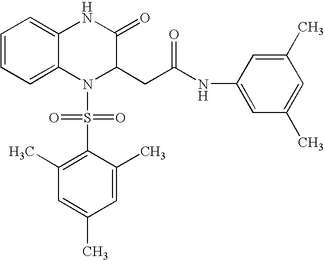 | 6% | Rt = 4.55 min; MS (ESIpos): 492 [M + H]+ | A |
| 119 | 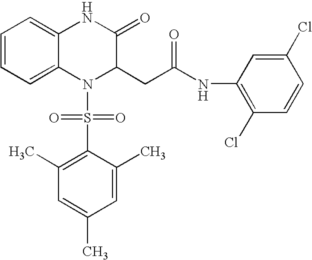 | 14% | Rt = 4.69 min; MS (ESIpos): 533 [M + H]+ | A |
| 120 | 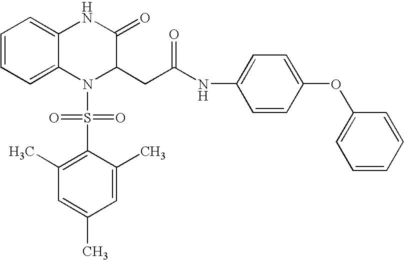 | 68% | Rt = 4.71 min; MS (ESIpos): 556 [M + H]+ | A |
| 121 | 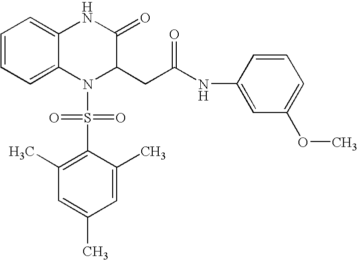 | 63% | Rt = 4.21 min; MS (ESIpos): 494 [M + H]+ | A |
| 122 | 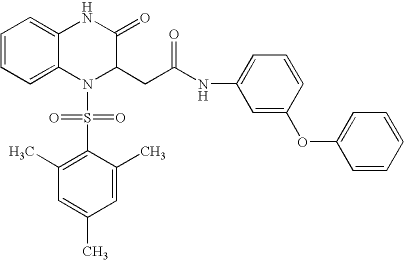 | 69% | Rt = 4.76 min; MS (ESIpos): 556 [M + H]+ | A |
| 123 | 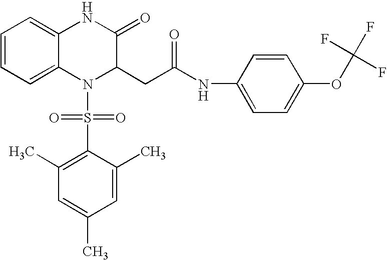 | 21% | Rt = 4.67 min; MS (ESIpos): 548 [M + H]+ | A |
| 124 | 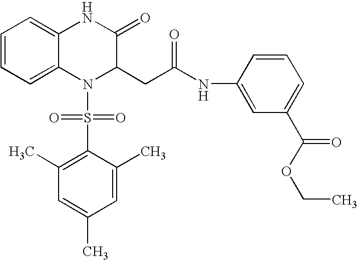 | 10% | Rt = 4.40 min; MS (ESIpos): 536 [M + H]+ | A |
| 125 | 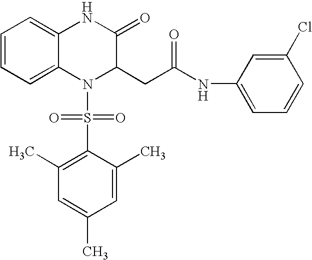 | 31% | Rt = 4.53 min; MS (ESIpos): 499 [M + H]+ | A |
| 126 | 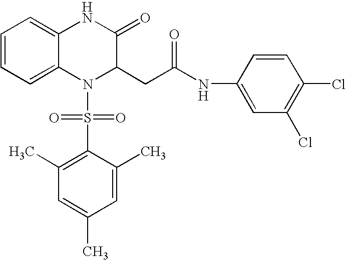 | 25% | Rt = 4.77 min; MS (ESIpos): 533 [M + H]+ | A |
| 127 | 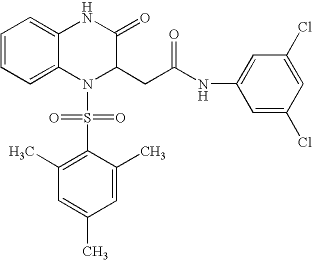 | 11% | Rt = 4.91 min; MS (ESIpos): 533 [M + H]+ | A |
| 128 | 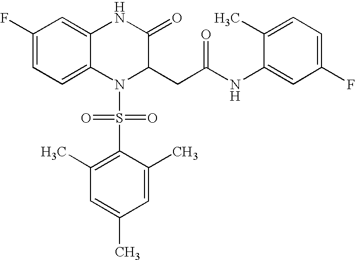 | 43% | Rt = 2.73 min; MS (ESIpos): 514 [M + H]+ | B |
| 129 | 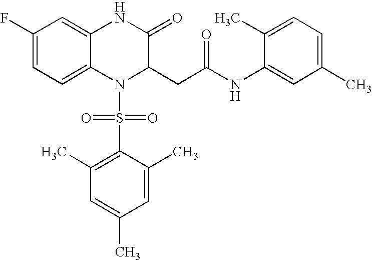 | 87% | Rt = 2.D2978 min; MS (ESIpos): 510 [M + H]+ | B |
| 130 | 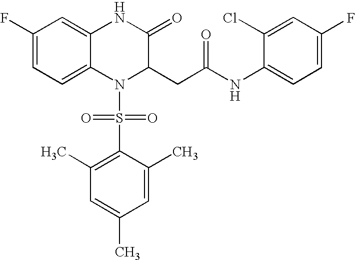 | 68% | Rt = 2.80 min; MS (ESIpos): 534 [M + H]+ | B |
| 131 | 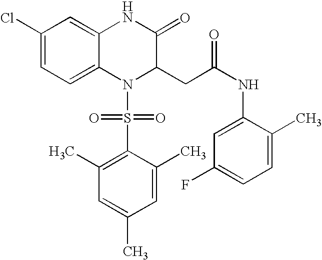 | 29% | Rt = 2.65 min; MS (ESIpos): 530 [M + H]+ | B |

General Method for the Sulfonylation of N-benzyl-2-(3-oxo-1,2,3,4-tetrehydro-2-quinoxalinyl)acetamide (Example I):

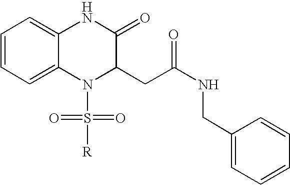

A solution of 30.7 mg (0.10 mmol) of N-benzyl-2-(3-oxo-1,2,3,4-tetrahydro-2-quinoxalinyl)acetamide (example I) and 0.21 inmol of the sulfonyl chloride in 3 ml of pyridine is stirred at 80° C. in an argon atmosphere overnight. The solvent is distilled off in vacuo, and the residue is purified by a preparative RP-HPLC (Kromasil 100 C18, 5 μm; 50×20 mm; Gradient: acetonitrile/water 15:85 to 90:10, room temperature).

Examples 132-159 listed in table 4 are obtained in accordance with this method:

TABLE 4

| | | | | LC/MS |
|---|---|---|---|---|
| Example | Structure | Yield | LC-MS | method |
| 132 | 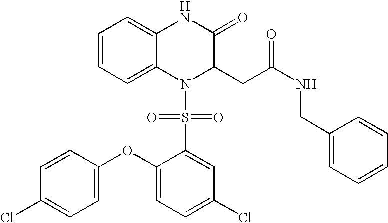 | 9% | Rt = 4.54 min; MS (ESIpos): 597 [M + H]+ | A |
| 133 | 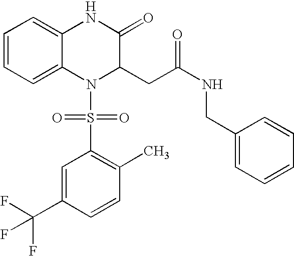 | 3% | Rt = 4.14 min; MS (ESIpos): 518 [M + H]+ | A |
| 134 | 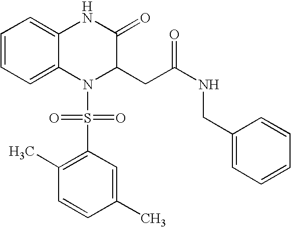 | 11% | Rt = 3.90 min; MS (ESIpos): 464 [M + H]+ | A |
| 135 | 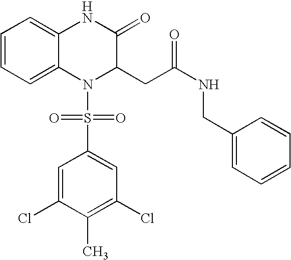 | 2% | Rt = 4.15 min; MS (ESIpos): 519 [M + H]+ | A |
| 136 | 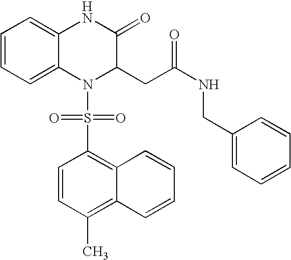 | 3% | Rt = 4.05 min; MS (ESIpos): 500 [M + H]+ | A |
| 137 | 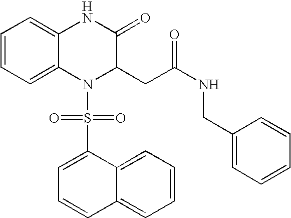 | 23% | Rt = 3.91 min; MS (ESIpos): 486 [M + H]+ | A |
| 138 | 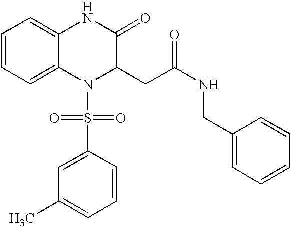 | 5% | Rt = 3.74 min; MS (ESIpos): 450 [M + H]+ | A |
| 139 | 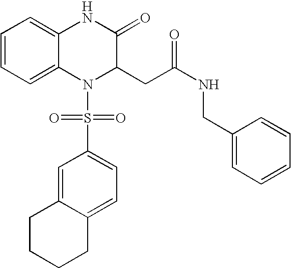 | 2% | Rt = 4.11 min; MS (ESIpos): 490 [M + H]+ | A |
| 140 | 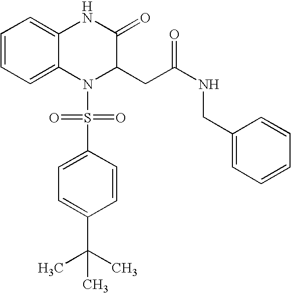 | 19% | Rt = 4.24 min; MS (ESIpos): 492 [M + H]+ | A |
| 141 | 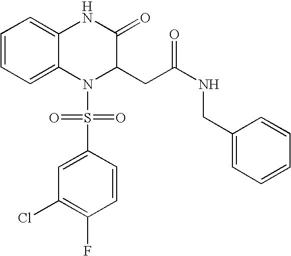 | 14% | Rt = 3.96 min; MS (ESIpos): 488 [M + H]+ | A |
| 142 | 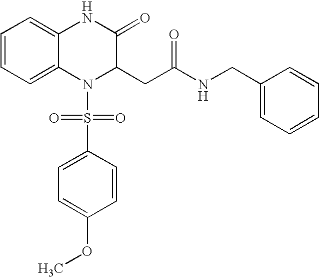 | 6% | Rt = 3.66 min; MS (ESIpos): 466 [M + H]+ | A |
| 143 | 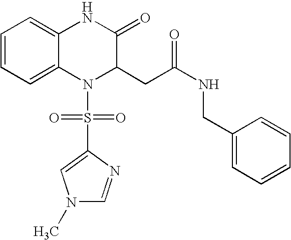 | 9% | Rt = 3.02 min; MS (ESIpos): 440 [M + H]+ | A |
| 144 | 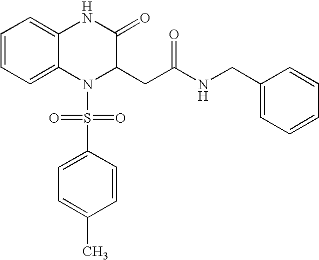 | 10% | Rt = 3.75 min; MS (ESIpos): 450 [M + H]+ | A |
| 145 | 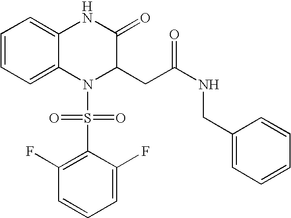 | 14% | Rt = 3.62 min; MS (ESIpos): 472 [M + H]+ | A |
| 146 | 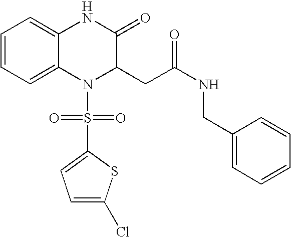 | 2% | Rt = 3.87 min; MS (ESIpos): 476 [M + H]+ | A |
| 147 | 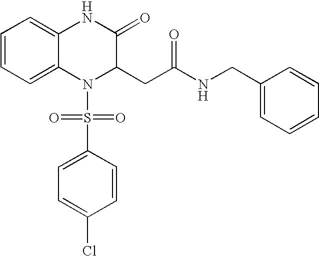 | 14% | Rt = 3.87 min; MS (ESIpos): 470 [M + H]+ | A |
| 148 | 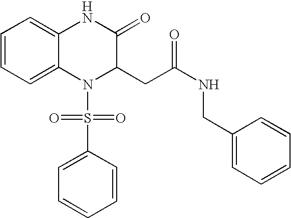 | 43% | Rt = 3.59 min; MS (ESIpos): 436 [M + H]+ | A |
| 149 | 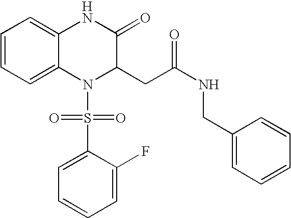 | 4% | Rt = 3.60 min; MS (ESIpos): 454 [M + H]+ | A |
| 150 | 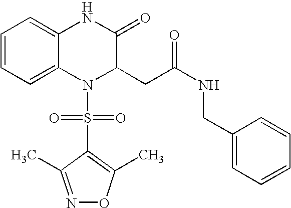 | 3% | Rt = 3.64 min; MS (ESIpos): 455 [M + H]+ | A |
| 151 | 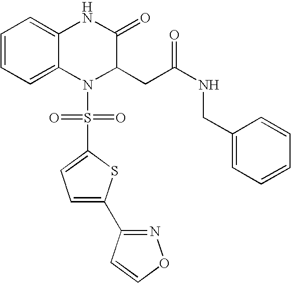 | 27% | Rt = 3.77 min; MS (ESIpos): 509 [M + H]+ | A |
| 152 | 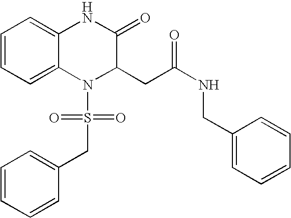 | 21% | Rt = 3.74 min; MS (ESIpos): 450 [M + H]+ | A |
| 153 | 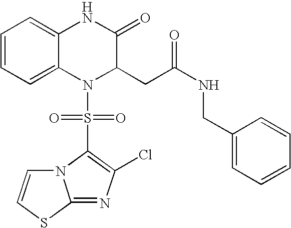 | 31% | Rt = 3.57 min; MS (ESIpos): 517 [M + H]+ | A |
| 154 | 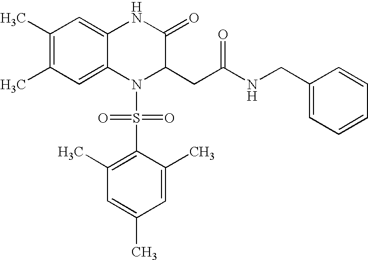 | 42% | Rt = 2.71 min; MS (ESIpos): 506 [M + H]+ | B |
| 155 | 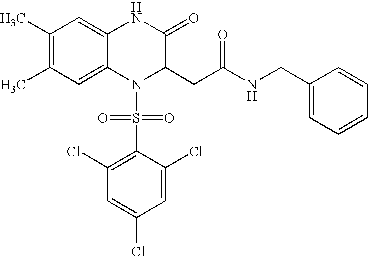 | 12% | Rt = 4.4 min; MS (ESIpos): 566 [M + H]+ | A |
| 156 | 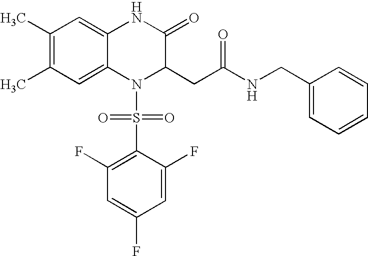 | 7% | Rt = 4.01 min; MS (ESIpos): 518 [M + H]+ | A |
| 157 | 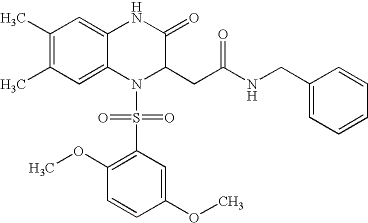 | 56% | Rt = 3.9 min; MS (ESIpos): 524 [M + H]+ | A |
| 158 | 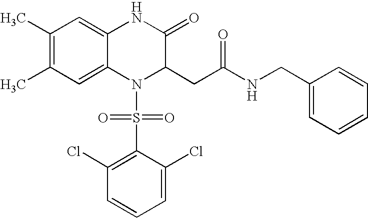 | 7% | Rt = 4.13 min; MS (ESIpos): 532 [M + H]+ | A |
| 159 | 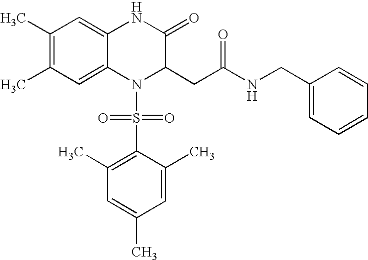 | 5% | Rt = 4.38 min; MS (ESIpos): 514 [M + H]+ | A |

General Method for the Sulfonylation of N-cycloheptyl-2-(oxo-1,2,3,4-tetrahydro-2-quinoxalinyl)acetamide (example VIII):

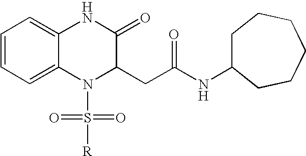

A solution of 100 mg (0.33 mmol) of N-cycloheptyl-2-(oxo-1,2,3,4-tetrahydro-2-quinoxalinyl)acetamide and 0.4 mmol of the sulfonyl chloride in 59 μl of pyridine and 3 ml of acetonitrile is stirred at 60° C. in an argon atmosphere overnight. The solvent is distilled off in vacuo, and the residue is purified by a preparative RP-HPLC (Kromasil 100 C18, 5 μm; 50×20 mm; Gradient: acetonitrile/water 15:85 to 90:10, room temperature).

Examples 160-180 listed in table 5 are obtained in accordance with this method:

TABLE 5

| | | | | LC/MS |
|---|---|---|---|---|
| Example | Structure | Yield | LC-MS | method |
| 160 | 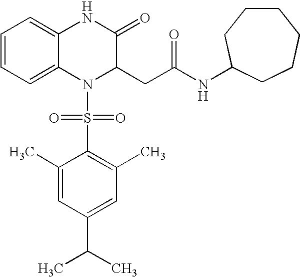 | 38% | Rt = 2.92 min; MS (ESIpos): 512 [M + H]+ | B |
| 161 | 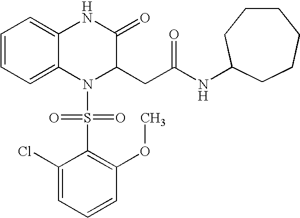 | 47% | Rt = 2.56 min; MS (ESIpos): 506 [M + H]+ | B |
| 162 | 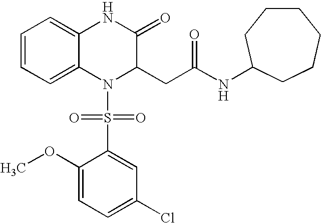 | 31% | Rt = 2.62 min; MS (ESIpos): 506 [M + H]+ | B |
| 163 | 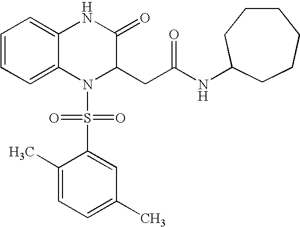 | 59% | Rt = 2.69 min; MS (ESIpos): 470 [M + H]+ | B |
| 164 | 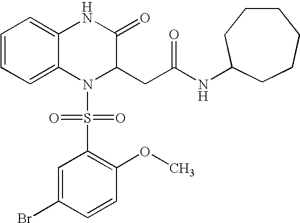 | 48% | Rt = 2.67 min; MS (ESIpos): 550 [M + H]+ | B |
| 165 | 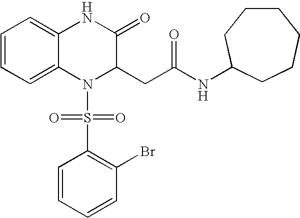 | 24% | Rt = 2.60 min; MS (ESIpos): 520 [M + H]+ | B |
| 166 | 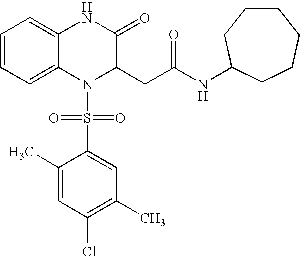 | 37% | Rt = 2.83 min; MS (ESIpos): 504 [M + H]+ | B |
| 167 | 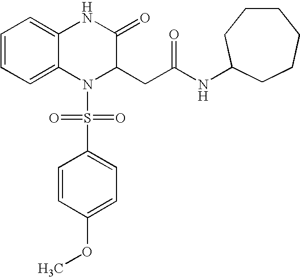 | 50% | Rt = 2.53 min; MS (ESIpos): 472 [M + H]+ | B |
| 168 | 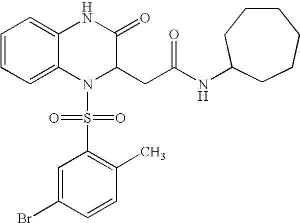 | 34% | Rt = 2.72 min; MS (ESIpos): 534 [M + H]+ | B |
| 169 | 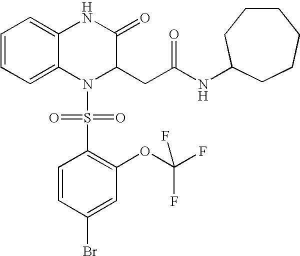 | 6% | Rt = 2.79 min; MS (ESIpos): 604 [M + H]+ | B |
| 170 | 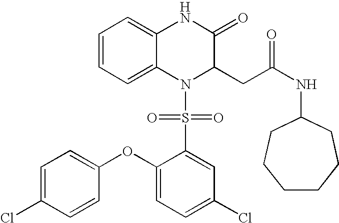 | 12% | Rt = 4.9 min; MS (ESIpos): 603 [M + H]+ | A |
| 171 | 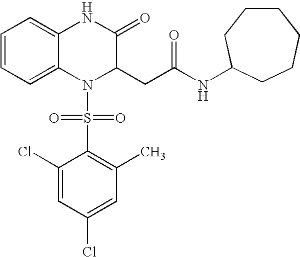 | 9% | Rt = 4.D185 min; MS (ESIpos): 525 [M + H]+ | A |
| 172 | 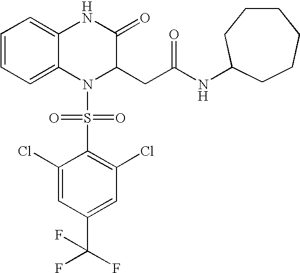 | 14% | Rt = 4.6 min; MS (ESIpos): 579 [M + H]+ | A |
| 173 | 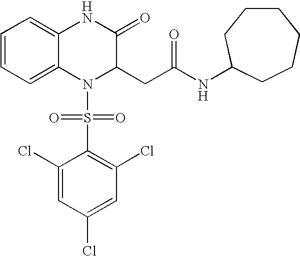 | 17% | Rt = 4.5 min; MS (ESIpos): 545 [M + H]+ | A |
| 174 | 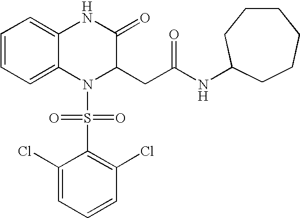 | 15% | Rt = 4.2 min; MS (ESIpos): 511 [M + H]+ | A |
| 175 | 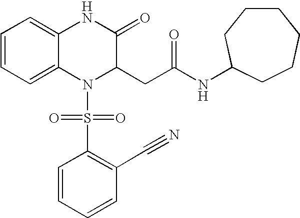 | 9% | Rt = 3.8 min; MS (ESIpos): 467 [M + H]+ | A |
| 176 | 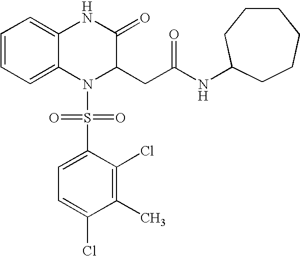 | 12% | Rt = 4.5 min; MS (ESIpos): 524 [M + H]+ | A |
| 177 | 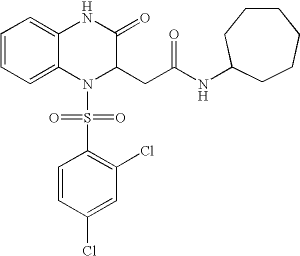 | 6% | Rt = 4.3 min; MS (ESIpos): 511 [M + H]+ | A |
| 178 | 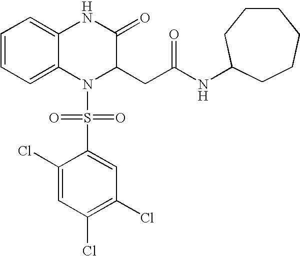 | 17% | Rt = 4.6 min; MS (ESIpos): 545 [M + H]+ | A |
| 179 | 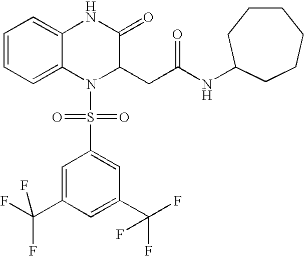 | 13% | Rt = 4.7 min; MS (ESIpos): 578 [M + H]+ | A |
| 180 | 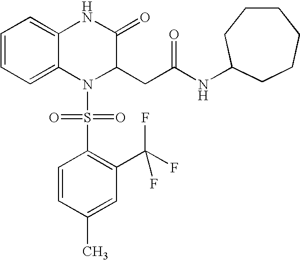 | 26% | Rt = 2.78 min; MS (ESIpos): 524 [M + H]+ | B |

### BEISPIEL 181

### N-Phenylmethyl-2-[1-(mesitylsulfonyl)-3-oxo-1,2,3,4-tetrahydro-2-quinoxalinyl]acetamide

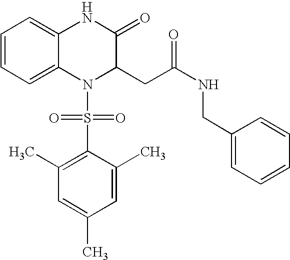

1.02 g (3.44 mmol) of N-phenylmethyl-2-(3-oxo-1,2,3,4-tetrahydro-2-quinoxalinyl)acetamide (example I) are suspended in about 10 ml of acetonitrile, and 3.80 g (17.4 mmol) of 2,4,6-trimethylphenylsulfonyl chloride and 523 mg (3.78 mmol) of potassium carbonate are added. The mixture is heated to reflux under an argon atmosphere for 3 hours. The mixture is then allowed to cool, and the contents of the flask are poured into water. The resulting brown solid is removed and triturated with diethyl ether. The beige crude product is purified fuirther by chromatography on silica gel (mobile phase: 1:1 dichloromethane/diethyl ether). This results in 770 mg (47%) of the target compound as a pale yellowish solid.

¹H-NMR (200 MHz, DMSO-d₆): δ=2.07-2.41 (m, 11H), 4.01-4.35 (ABX system, AB part, 2H), 4.81 (dd, 1H), 6.89-7.39 (m, 11H), 8.35 (t, 1H), 10.65 (s, 1H).

MS (DCI, NH₃): m/z=495 [M+NH₄]⁺, 478 [M+H]⁺.

### EXAMPLE 182

### N-(2-Methoxyphenyl)-2-[1-(mesitylsulfonyl)-3-oxo-1,2,3,4-tetrahydro-2-quinoxalinyl]acetamide

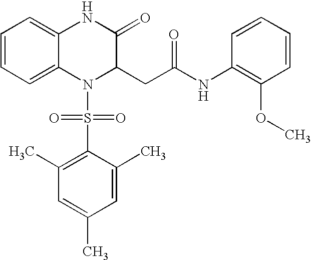

This compound is obtained in analogy to the method of example 86 from 642 mg (2.06 mmol) of N-(2-methoxyphenyl)-2-(3-oxo-1,2,3,4-tetrahydro-2-quinoxalinyl)acetamide (example II) and 2.25 g (10.3 mmol) of 2,4,6-trimethylphenylsulfonyl chloride by heating to reflux for 2 hours and stirring the initially obtained brownish red solid with acetone.

Yield: 804 mg (79%) of a colorless solid.

¹H-NMR (200 MHz, DMSO-d₆): δ=2.25 (s, 3H), 2.32 (s, 6H), 2.56 (d, 2H), 3.76 (s, 3H), 4.82 (t, 1H), 6.70-7.38 (m, 9H), 7.91 (d, 1H), 9.13 (s, 1H), 10.67 (s, 1H).

MS (DCI, NH₃): m/z=511 [M+NH₄]⁺, 494 [M+H]⁺.

### EXAMPLE 183 AND EXAMPLE 184

### 2S-N-Cycloheptyl-2-[1-(mesitylsulfonyl)-3-oxo-1,2,3,4-tetrahydro-2-quinoxalinyl]acetamide and 2R-N-cycloheptyl-2-[1-(mesitylsulfonyl)-3-oxo-1,2,3,4-tetrahydro-2-quinoxalinyl]acetamide

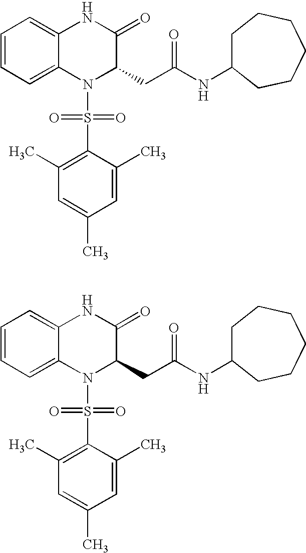

4 g of N-cycloheptyl-2RS-[1-(mesitylsulfonyl)-3-oxo-1,2,3,4-tetrahydro-2-quinoxalinyl]acetamide (example 1) are separated into the enantiomers by means of a chiral HPLC.

Description of method:

| | |
|---|---|
| Sample preparation: | 4 g dissolved in 750 ml of ethyl acetate |
| Sample loading: | 400 mg every 36 min |
| Flow rate: | 40 ml/min |
| Wave length: | 254 nM |
| Solvent: | ethyl acetate |
| Packing material: | 6784 (600*30); LNW 2951; N-MA-L-leu-2,4- |
| | dimethylpentyl-amide |

The following are obtained:

S-N-Cycloheptyl-2-[1-(mesitylsulfonyl)-3-oxo-1,2,3,4-tetrahydro-2-quinoxalinyl]acetamide: 1.95 g (Rₜ=20.292 min).

Specific rotation [α]²⁰_{D}=−88.6° (c=0.485; MeOH).

and R-N-cycloheptyl-2-[1-(mesitylsulfonyl)-3-oxo-1,2,3,4-tetrahydro-2-quinoxalinyl]acetamide: 1.75 g (Rₜ=32.925 min).

Specific rotation [α]²⁰_{D}=+95.8° (c=0.514; MeOH).

(The concentration c stated in connection with the specific rotation is defined as the amount of substance (in g) per 100 ml of solution. Path length: 100 mm).

TABLE 6

| | | MW | Yield | | LC/MS |
|---|---|---|---|---|---|
| Example | Structure | [g/mol] | % | LC-MS/MS | method |
| 185 | 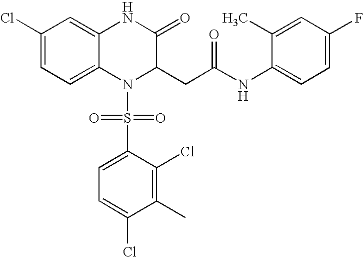 | 570.85 | 51 | MS(ESIpos): m/z = 570 (M + H) Rt = 2.84 min | B |
| 186 | 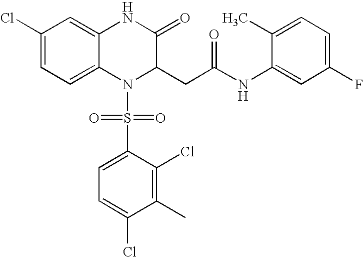 | 570.85 | 42 | MS(ESIpos): m/z = 570 (M + H) Rt = 2.89 min | B |
| 187 | 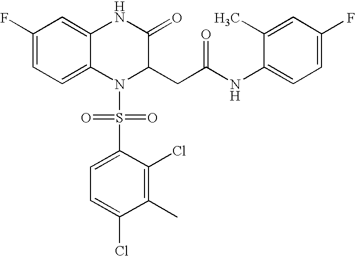 | 554.40 | 66 | MS(ESIpos): m/z = 554 (M + H) Rt = 2.78 min | B |
| 188 | 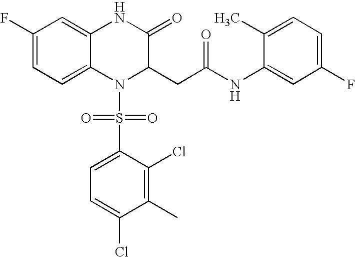 | 554.40 | 16 | MS(ESIpos): m/z = 554 (M + H) Rt = 2.83 min | B |
| 189 | 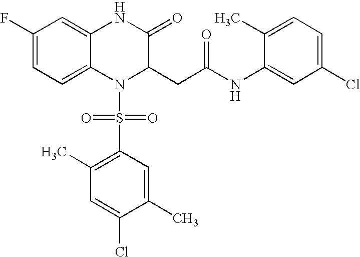 | 550.44 | 37 | MS(ESIpos): m/z = 550 (M + H) Rt = 2.91 min | B |
| 190 | 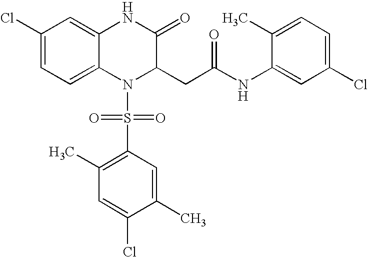 | 566.89 | 44 | MS(ESIpos): m/z = 566 (M + H) Rt = 3.02 min | B |
| 191 | 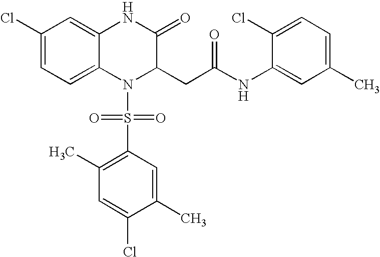 | 566.89 | 12 | MS(ESIpos): m/z = 566 (M + H) Rt = 3.07 min | B |
| 192 | 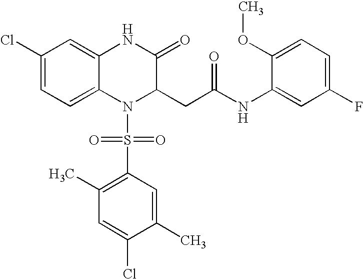 | 566.43 | 60 | MS(DCI): m/z = 583 (M + NH4) | |
| 193 | 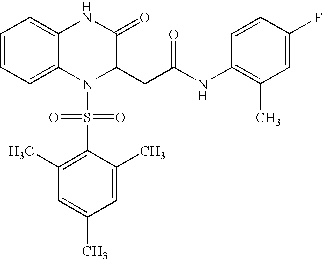 | 495.57 | 42 | MS(ESIpos): m/z = 496 (M + H) Rt = 2.71 min | B |
| 194 | 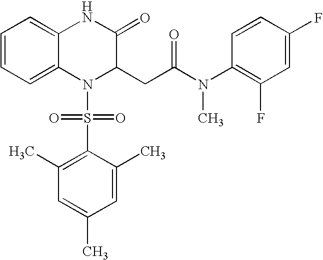 | 513.56 | 44 | MS(ESIpos): m/z = 514 (M + H) Rt = 2.72 min | B |
| 195 | 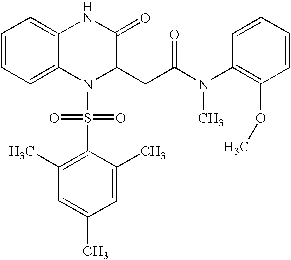 | 507.61 | 38 | MS(ESIpos): m/z = 508 (M + H) Rt = 2.63 min | B |
| 196 | 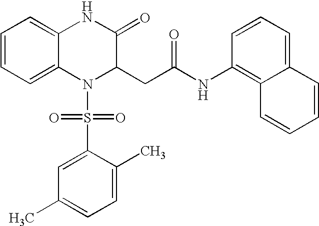 | 499.59 | 31 | MS(ESIpos): m/z = 500 (M + H) Rt = 4.30 min | A |
| 197 | 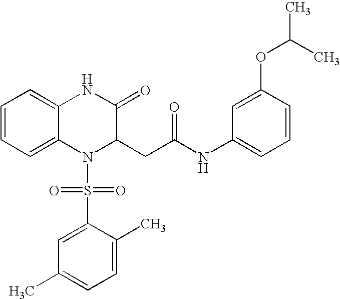 | 507.61 | 18 | MS(ESIpos): m/z = 508 (M + H) Rt = 4.47 min | A |
| 198 | 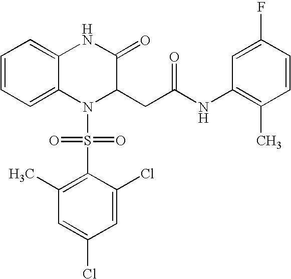 | 536.41 | 13 | MS(ESIpos): m/z = 536 (M + H) Rt = 4.52 min | A |
| 199 | 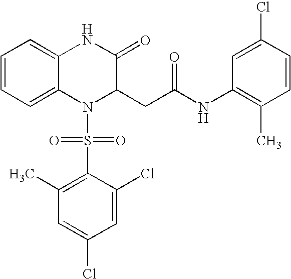 | 512.03 | 29 | MS(ESIpos): m/z = 512 (M + H) Rt = 2.84 min | B |
| 200 | 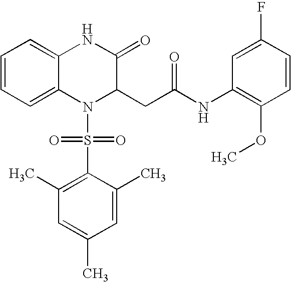 | 511.57 | 34 | MS(ESIpos): m/z = 512 (M + H) Rt = 2.75 min | B |
| 201 | 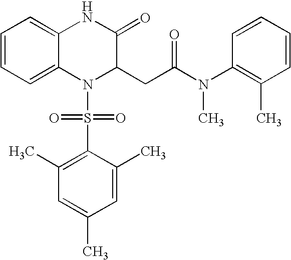 | 491.61 | 26 | MS(ESIpos): m/z = 492 (M + H) Rt = 2.72 min | B |
| 202 | 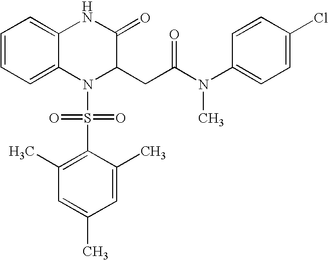 | 512.03 | 27 | MS(ESIpos): m/z = 512 (M + H) Rt = 2.77 min | B |
| 203 | 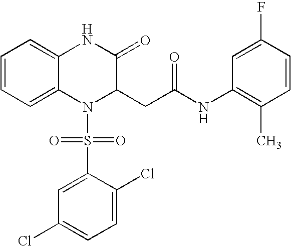 | 522.38 | 20 | MS(ESIpos): m/z = 522 (M + H) Rt = 4.34 min | A |
| 204 | 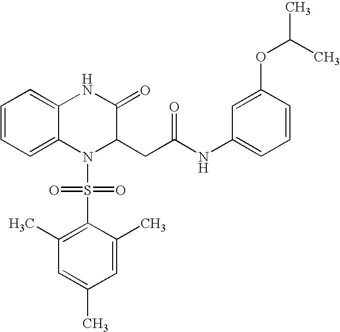 | 521.63 | 20 | MS(ESIpos): m/z = 522 (M + H) Rt = 2.85 min | B |
| 205 | 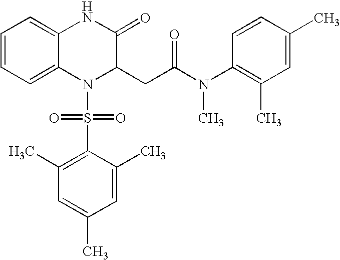 | 505.64 | 24 | MS(ESIpos): m/z = 506 (M + H) Rt = 4.59 min | A |
| 206 | 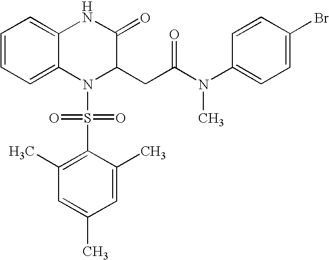 | 556.48 | 25 | MS(ESIpos): m/z = 556 (M + H) Rt = 2.82 min | B |
| 207 | 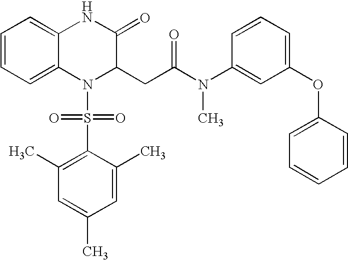 | 569.68 | 6 | MS(ESIpos): m/z = 570 (M + H) Rt = 2.91 min | B |
| 208 | 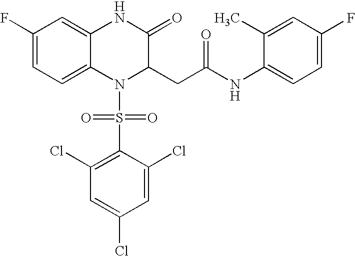 | 574.82 | 31 | MS(DCI): m/z = 593 (M + NH4) | |
| 209 | 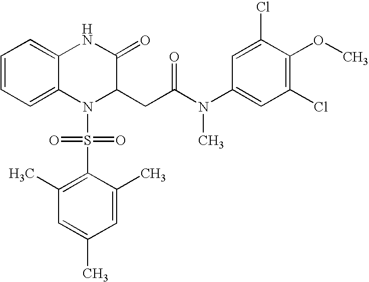 | 576.50 | 4 | MS(ESIpos): m/z = 576 (M + H) Rt = 2.86 min | B |
| 210 | 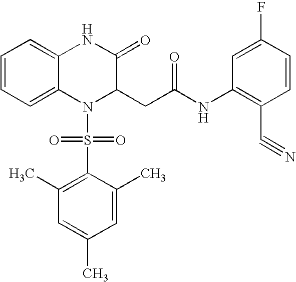 | 506.56 | 68 | MS(ESIpos): m/z = 507 (M + H) Rt = 4.43 min | A |
| 211 | 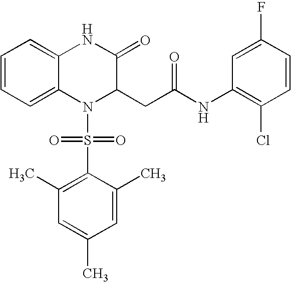 | 515.99 | 2 | MS(ESIpos): m/z = 516 (M + H) Rt = 4.54 min | A |
| 212 | 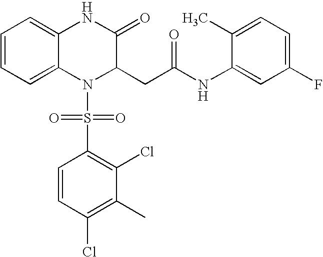 | 536.41 | 7 | MS(ESIpos): m/z = 536 (M + H) Rt = 4.43 min | A |
| 213 | 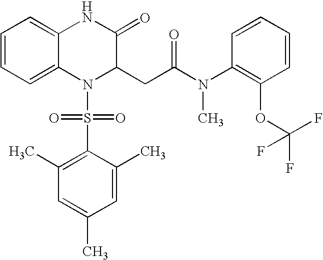 | 561.58 | 28 | MS(ESIpos): m/z = 562 (M + H) Rt = 4.70 min | A |
| 214 | 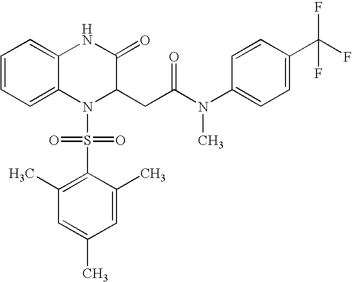 | 545.58 | 53 | MS(ESIpos): m/z = 546 (M + H) Rt = 4.63 min | A |
| 215 | 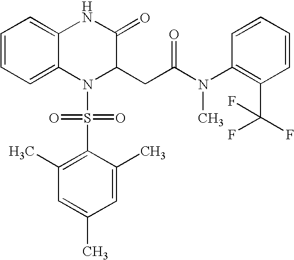 | 545.58 | 12 | MS(ESIpos): m/z = 546 (M + H) Rt = 4.66 min | A |
| 216 | 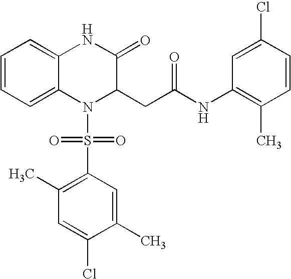 | 532.45 | 22 | MS(ESIneg): m/z = 530 (M − H) Rt = 4.71 min | A |
| 217 | 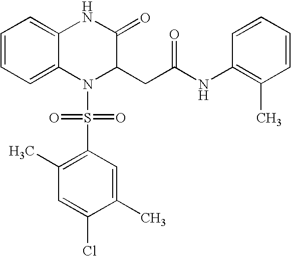 | 498.00 | 45 | MS(ESIpos): m/z = 498 (M + H) | |
| 218 | 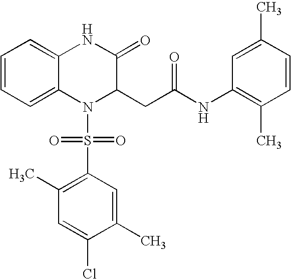 | 512.03 | 71 | MS(ESIneg): m/z = 510 (M − H) Rt = 4.57 min | A |
| 219 | 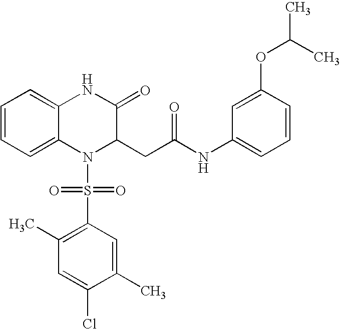 | 542.05 | 89 | MS(ESIpos): m/z = 542 (M + H) Rt = 4.74 min | A |
| 220 | 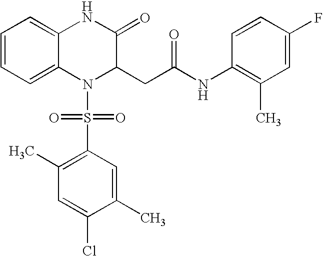 | 515.99 | 37 | MS(ESIpos): m/z = 516 (M + H) Rt = 4.44 min | A |
| 221 | 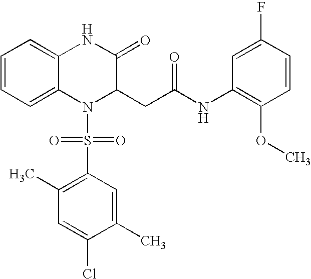 | 531.99 | 50 | MS(ESIneg): m/z = 530 (M − H) Rt = 4.56 min | A |
| 222 | 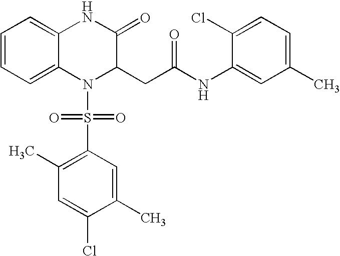 | 532.45 | 8 | MS(ESIpos): m/z = 532 (M + H) Rt = 4.72 min | A |
| 223 | 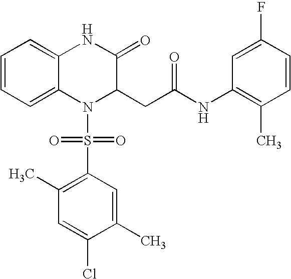 | 515.99 | 29 | MS(ESIpos): m/z = 516 (M + H) Rt = 4.53 min | A |
| 224 | 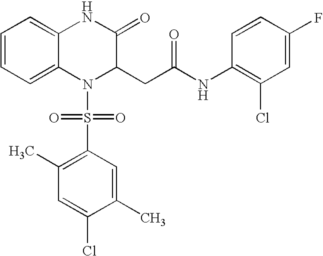 | 536.41 | 22 | MS(ESIpos): m/z = 536 (M + H) Rt = 4.58 min | A |
| 225 | 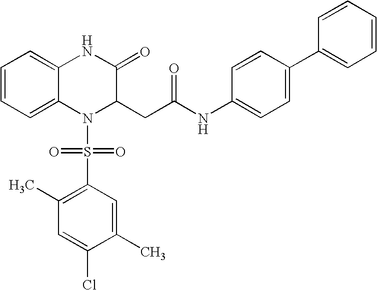 | 560.07 | 76 | MS(ESIpos): m/z = 560 (M + H) Rt = 4.91 min | A |
| 226 | 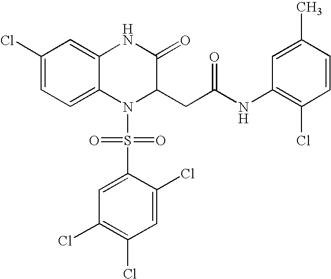 | 607.73 | 7 | MS(ESIpos): m/z = 606 (M + H) Rt = 5.00 min | A |
| 227 | 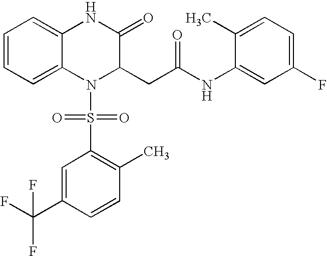 | 535.52 | 42 | MS(ESIpos): m/z = 536 (M + H) | |
| 228 | 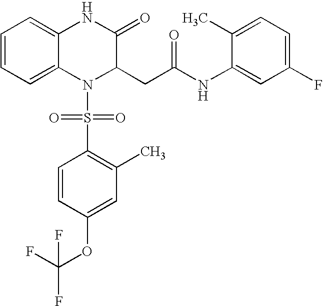 | 571.93 | 36 | MS(DCI): m/z = 589 (M + NH4) | |
| 229 | 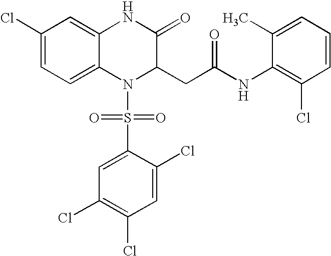 | 607.73 | 4 | MS(ESIpos): m/z = 605 (M + H) | |
| 230 | 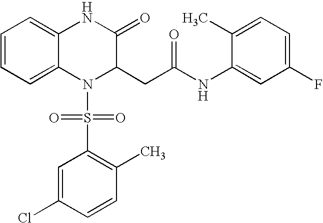 | 501.96 | 66 | MS(ESIpos): m/z = 502 (M + H) | |
| 231 | 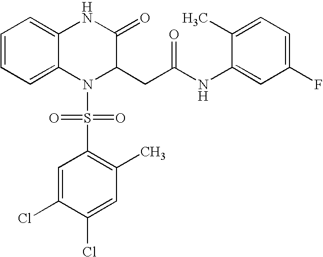 | 536.41 | 46 | MS(ESIpos): m/z = 536 (M + H) | |
| 232 | 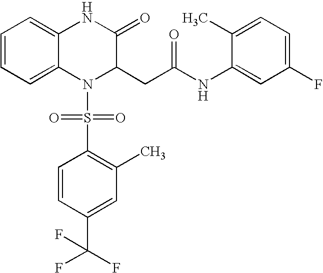 | 535.52 | 28 | MS(ESIpos): m/z = 536 (M + H) | |
| 233 | 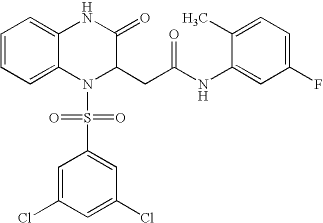 | 522.38 | 82 | MS(ESIpos): m/z = 522 (M + H) | |
| 234 | 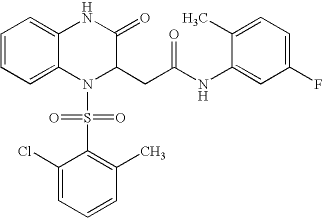 | 501.96 | 36 | MS(ESIpos): m/z = 502 (M + H) | |
| 235 | 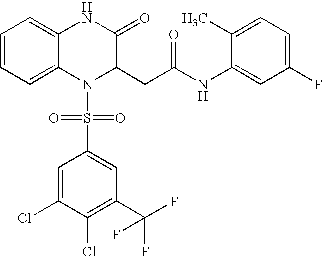 | 590.38 | 63 | MS(ESIpos): m/z = 590 (M + H) | |
| 236 | 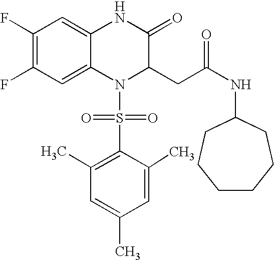 | 519.61 | 42.85 | LCMS: Rt =2.87 min MS(ESIpos): m/z = 520 (M + H) | B |
| 237 | 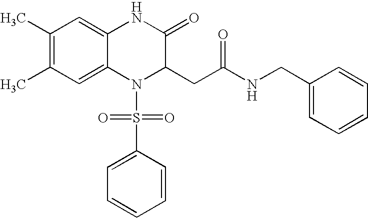 | 463.56 | 47.4 | LCMS: Rt =3.92 min MS(ESIpos): m/z = 464 (M + H) | A |
| 238 | 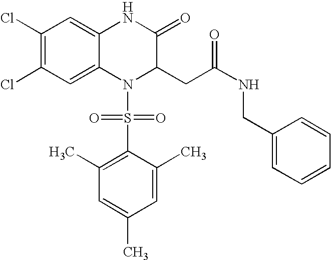 | 546.47 | 46.5 | LCMS: Rt =4.71 min MS(ESIpos): m/z = 448 (M + H) | A |
| 239 | 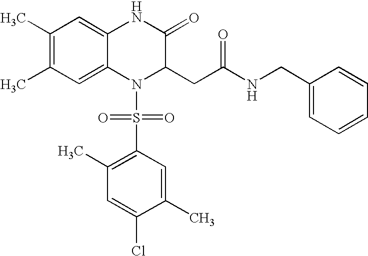 | 526.05 | 34.9 | LCMS: Rt =4.58 min MS(ESIpos): m/z = 526 (M + H) | A |
| 240 | 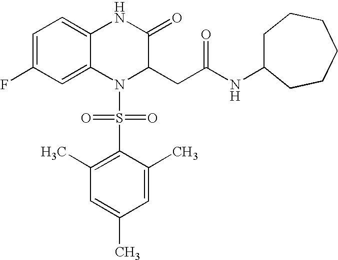 | 501.62 | 88.1 | LCMS: Rt =4.61 min MS(ESIpos): m/z = 502 (M + H) | A |
| 241 | 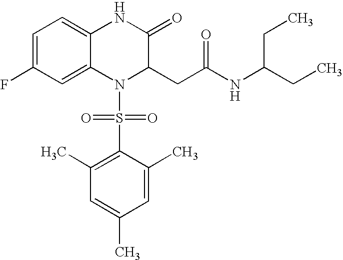 | 475.48 | 91 | LCMS: Rt =4.37 min MS(ESIpos): m/z = 476 (M + H) | A |
| 242 | 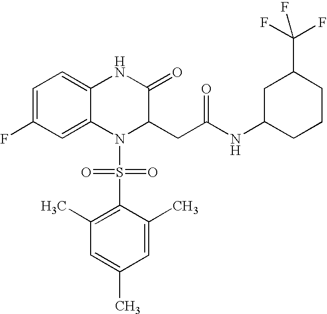 | 555.59 | 73 | LCMS: Rt =4.59 min MS(ESIpos): m/z = 556 (M + H) | A |
| 243 | 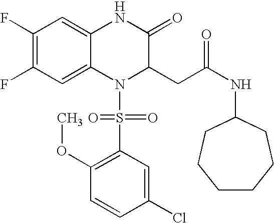 | 542.00 | 48 | LCMS: Rt =4.60 min MS(ESIpos): m/z = 542 (M + H) | A |
| 244 | 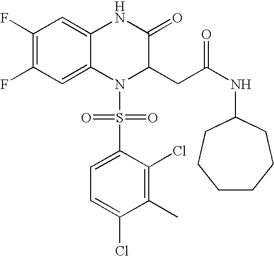 | 560.45 | 27.1 | LCMS: Rt =5.07 min MS(ESIpos): m/z = 560 (M + H) | B |
| 245 | 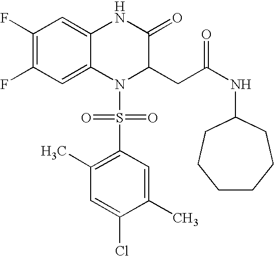 | 540.03 | 56 | LCMS: Rt =2.95 min MS(ESIpos): m/z = 540 (M + H) | B |
| 246 | 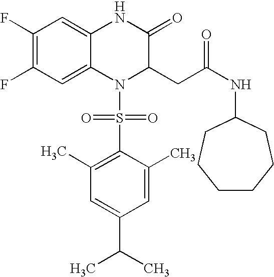 | 547.66 | 43 | LCMS: Rt =3.01 min MS(ESIpos): m/z = 548 (M + H) | B |
| 247 | 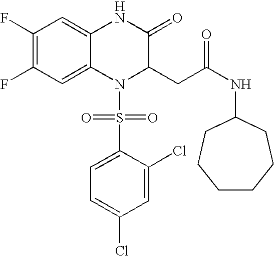 | 546.42 | 16.05 | LCMS: Rt =4.66 min MS(ESIpos): m/z = 546 (M + H) | A |
| 248 | 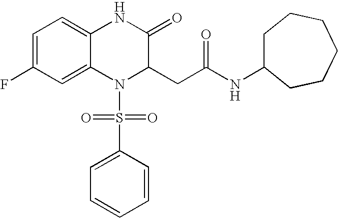 | 459.54 | 65.2 | LCMS: Rt =4.00 min MS(ESIpos): m/z = 460 (M + H) | A |
| 249 | 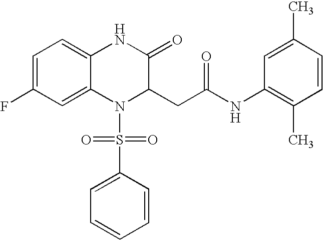 | 467.52 | 62 | LCMS: Rt =4.00 min MS(ESIpos): m/z = 468 (M + H) | A |
| 250 | 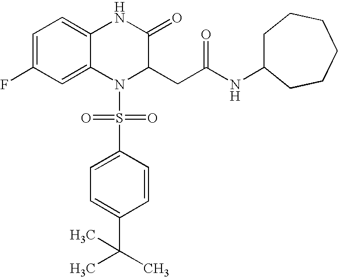 | 515.65 | 85 | LCMS: Rt =4.70 min MS(ESIpos): m/z = 416 (M + H) | A |
| 251 | 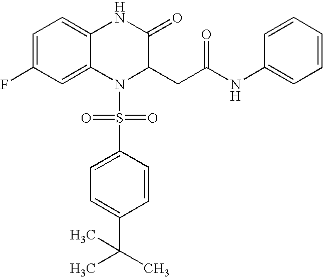 | 495.57 | 82 | LCMS: Rt =4.50 min MS(ESIpos): m/z = 496 (M + H) | A |
| 252 | 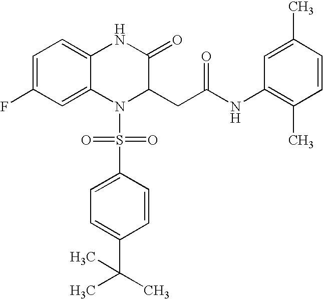 | 523.63 | 54.4 | LCMS: Rt =4.60 min MS(ESIpos): m/z = 524 (M + H) | A |
| 253 | 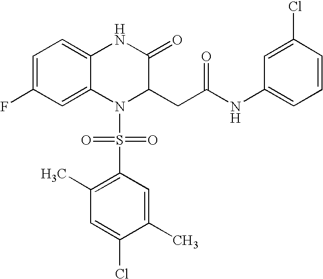 | 536.41 | 47.3 | LCMS: Rt =4.74 min MS(ESIpos): m/z = 537 (M + H) | A |
| 254 | 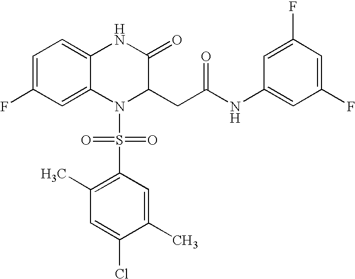 | 537.94 | 20.5 | LCMS: Rt =4.71 min MS(ESIpos): m/z = 538 (M + H) | A |
| 255 | 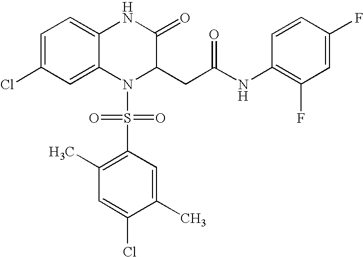 | 554.40 | 41.3 | LCMS: Rt =4.90 min MS(ESIpos): m/z = 554 (M + H) | A |
| 256 | 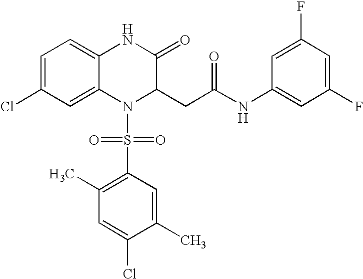 | 554.40 | 60.5 | LCMS: Rt =4.70 min MS(ESIpos): m/z = 554 (M + H) | A |
| 257 | 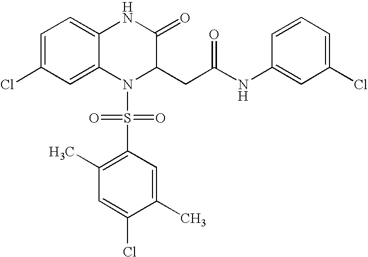 | 552.86 | 91.9 | LCMS: Rt =4.90 min MS(ESIpos): m/z = 554 (M + H) | A |
| 258 | 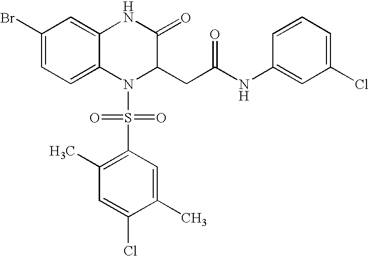 | 597.32 | 59.9 | LCMS: Rt =5.0 min MS(ESIpos): m/z = 598 (M + H) | A |
| 259 | 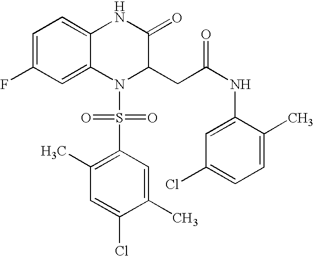 | 550.44 | 53 | LCMS: Rt =4.75 min MS(ESIpos): m/z = 550 (M + H) | A |
| 260 | 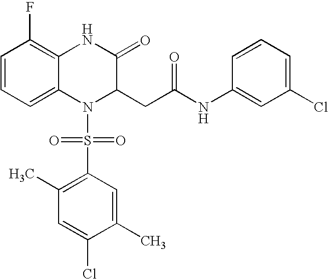 | 536.41 | 60.34 | LCMS: Rt =4.80 min MS(ESIpos): m/z = 538 (M + H) | A |
| 261 | 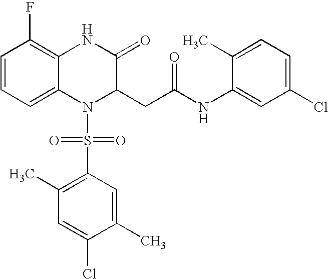 | 550.44 | 41.5 | LCMS: Rt =4.80 min MS(ESIpos): m/z = 550 (M + H) | A |
| 262 | 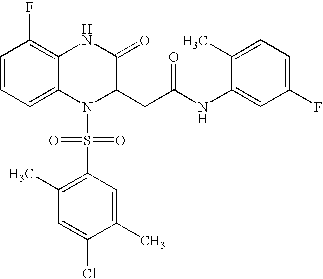 | 533.98 | 45.4 | LCMS: Rt =4.60 min MS(ESIpos): m/z = 534 (M + H) | A |
| 263 | 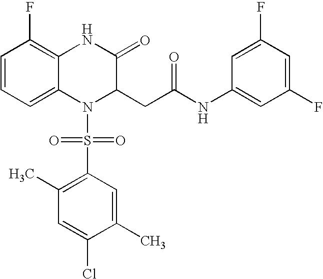 | 537.94 | 28 | LCMS: Rt =4.70 min MS(ESIpos): m/z = 538 (M + H) | A |
| 264 | 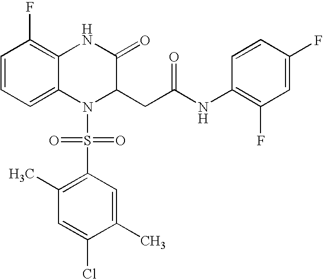 | 537.94 | 50.6 | LCMS: Rt =4.54 min MS(ESIpos): m/z = 538,2 (M + H) | A |
| 265 | 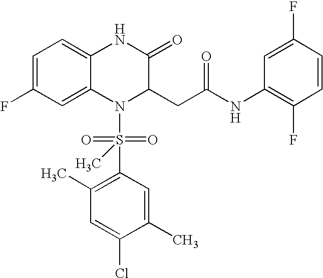 | 552.98 | 26 | LCMS: Rt =4.60 min MS(ESIpos): m/z = 538 (M + H) | A |
| 266 | 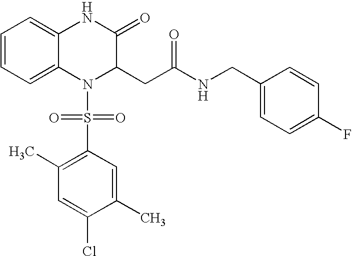 | 515.99 | 53 | LCMS: Rt =5.34 min MS(ESIpos): m/z = 538 (M + H) | A |
| 267 | 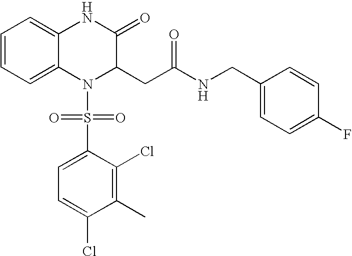 | 536.41 | 15 | LCMS: Rt =4.28 min MS(ESIpos): m/z = 536 (M + H) | A |
| 268 | 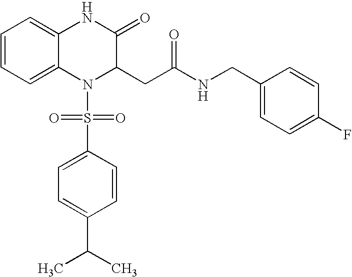 | 495.57 | 56.1 | LCMS: Rt =5.04 min MS(ESIpos): m/z = 496 (M + H) | A |
| 269 | 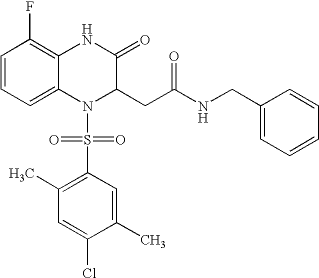 | 515.99 | 79.8 | LCMS: Rt =2.75 min MS(ESIpos): m/z = 516 (M + H) | B |
| 270 | 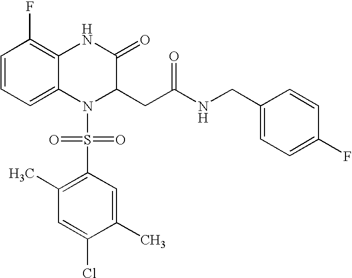 | 533.98 | 76 | LCMS: Rt =2.76 min MS(ESIpos): m/z = 534 (M + H) | B |
| 271 | 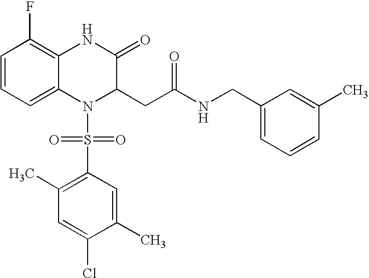 | 530.02 | 74.4 | LCMS: Rt =2.89 min MS(ESIpos): m/z = 530 (M + H) | B |
| 272 | 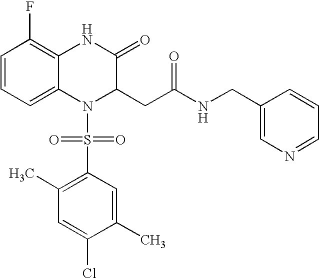 | 516.98 | 73 | LCMS: Rt =2.19 min MS(ESIpos): m/z = 517 (M + H) | B |
| 273 | 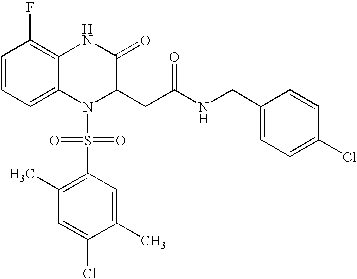 | 550.44 | 73.1 | LCMS: Rt =2.92 min MS(ESIpos): m/z = 550 (M + H) | B |
| 274 | 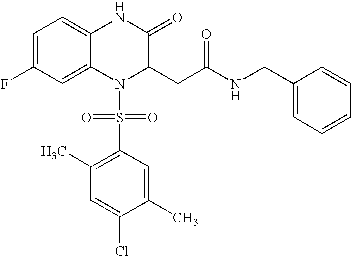 | 515.99 | 100 | LCMS: Rt =2.76 min MS(ESIpos): m/z = 516 (M + H) | B |
| 275 | 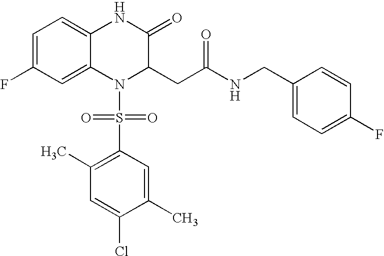 | 533.98 | 77.3 | LCMS: Rt =2.78 min MS(ESIpos): m/z = 534 (M + H) | B |
| 276 | 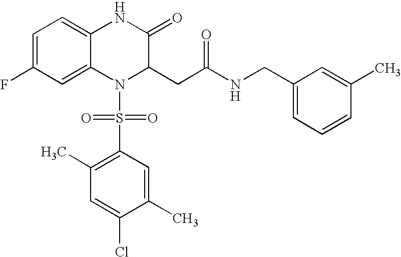 | 530.02 | 78.3 | LCMS: Rt =2.90 min MS(ESIpos): m/z = 530 (M + H) | B |
| 277 | 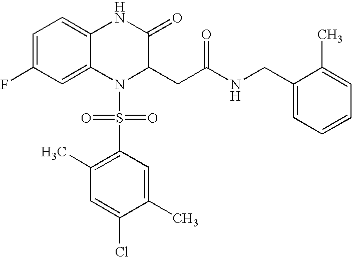 | 530.02 | 72.1 | LCMS: Rt =2.78 min MS(ESIpos): m/z = 530 (M + H) | B |
| 278 | 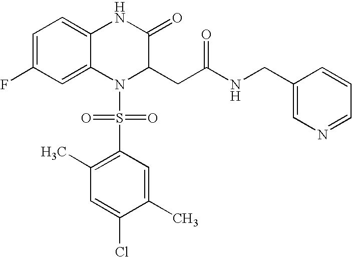 | 516.98 | 67 | LCMS: Rt =2.00 min MS(ESIpos): m/z = 517 (M + H) | B |
| 279 | 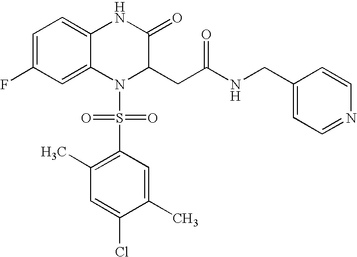 | 516.98 | 71 | LCMS: Rt =1.95 min MS(ESIpos): m/z = 517 (M + H) | B |
| 280 | 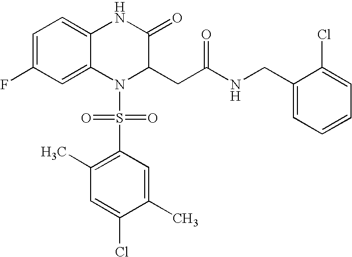 | 550.44 | 77.2 | LCMS: Rt =2.76 min MS(ESIpos): m/z = 550 (M + H) | B |
| 281 | 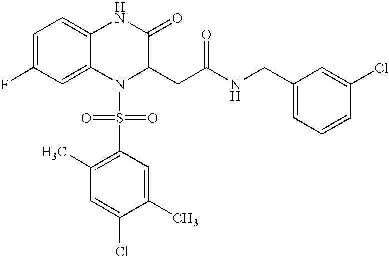 | 550.44 | 69.4 | LCMS: Rt =2.79 min MS(ESIpos): m/z = 550 (M + H) | B |
| 282 | 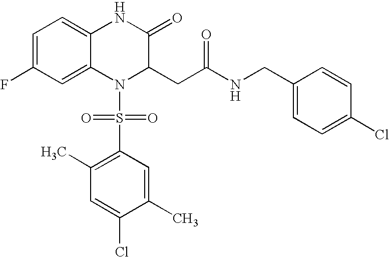 | 550.44 | 71.5 | LCMS: Rt =4.58 min MS(ESIpos): m/z = 550 (M + H) | A |
| 283 | 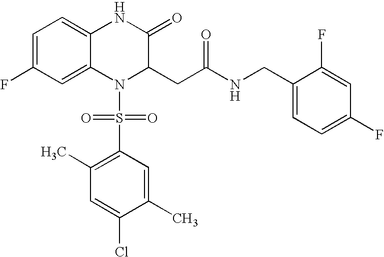 | 551.97 | 73.1 | LCMS: Rt =4.46 min MS(ESIpos): m/z = 551 (M + H) | A |
| 284 | 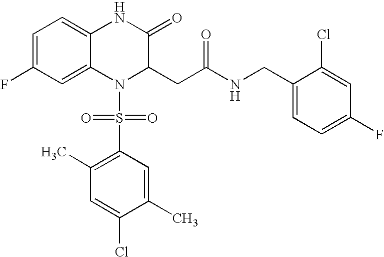 | 568.43 | 64.2 | LCMS: Rt =4.63 min MS(ESIpos): m/z = 568 (M + H) | A |
| 285 | 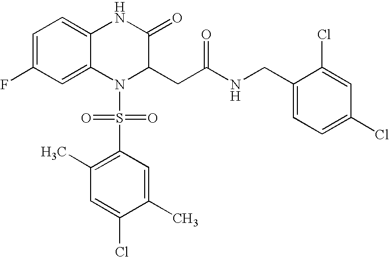 | 584.88 | 80.04 | LCMS: Rt =4.84 min MS(ESIpos): m/z = 586 (M + H) | A |
| 286 | 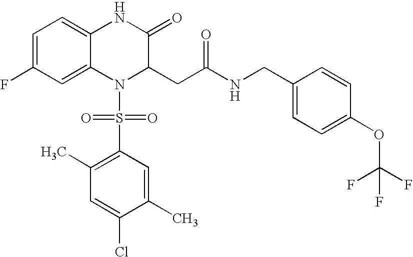 | 599.99 | 73.16 | LCMS: Rt =4.76 min MS(ESIpos): m/z = 600 (M + H) | A |
| 287 | 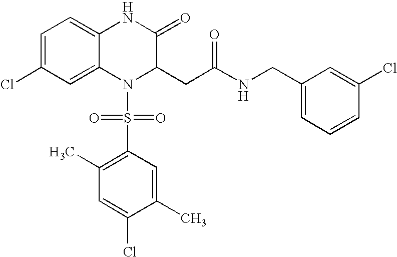 | 566.89 | 70.81 | LCMS: Rt =4.74 min MS(ESIpos): m/z = 566 (M + H) | A |
| 288 | 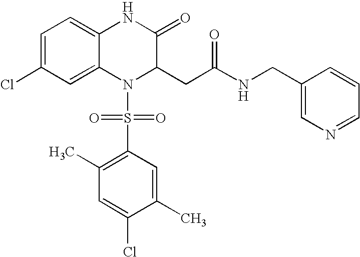 | 533.43 | 44.12 | LCMS: Rt =3.26 min MS(ESIpos): m/z = 533 (M + H) | A |
| 289 | 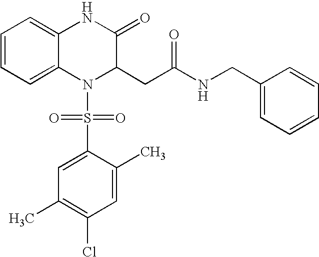 | 498.00 | 93.8 | MS(ESIpos): m/z = 498 (M + H) HPLC: Rt =4.74 min | C |
| 290 | 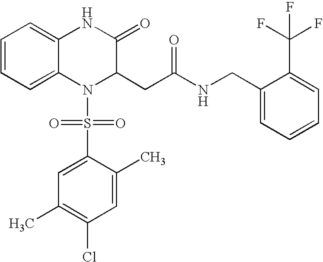 | 566.00 | 94 | MS(ESIpos): m/z = 566 (M + H) HPLC: Rt =4.98 min | C |
| 291 | 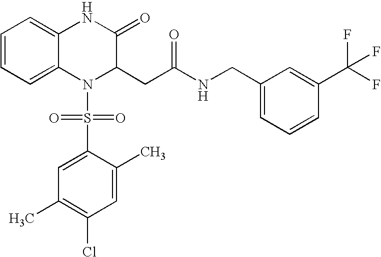 | 566.00 | 96 | MS(ESIpos): m/z = 566 (M + H) HPLC: Rt =4.97 min | C |
| 292 | 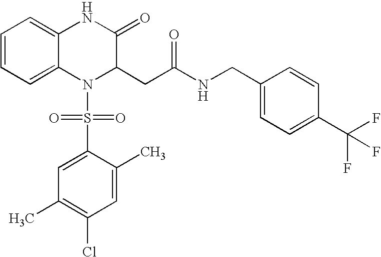 | 566.00 | 95 | MS(ESIpos): m/z = 566 (M + H) HPLC: Rt =4.98 min | C |
| 293 | 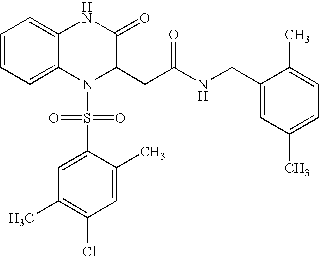 | 526.05 | 94 | MS(ESIpos): m/z = 526 (M + H) HPLC: Rt =4.97 min | C |
| 294 | 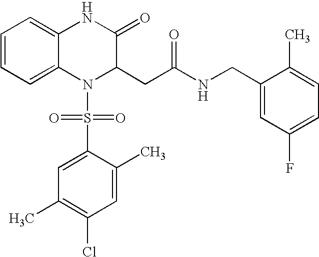 | 530.02 | 95 | MS(ESIpos): m/z = 530 (M + H) HPLC: Rt =4.85 min | C |
| 295 | 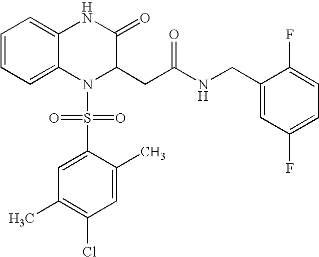 | 533.98 | 94 | MS(ESIpos): m/z = 534 (M + H) HPLC: Rt =4.77 min | C |
| 296 | 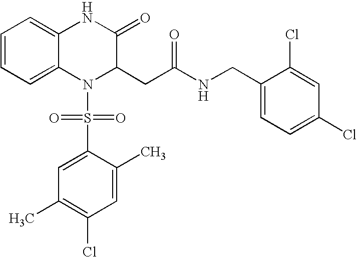 | 566.89 | 93 | MS(ESIpos): m/z = 566 (M + H) HPLC: Rt =5.12 min | C |
| 297 | 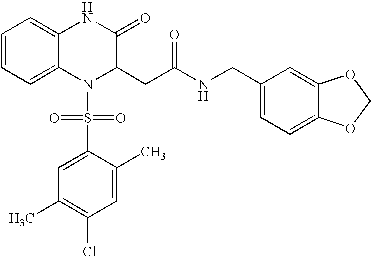 | 542.01 | 90 | MS(ESIpos): m/z = 542 (M + H) HPLC: Rt =4.64 min | C |
| 298 | 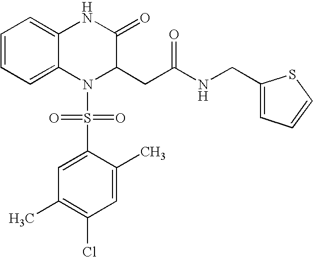 | 504.03 | 91 | MS(ESIpos): m/z = 504 (M + H) HPLC: Rt =4.66 min | C |
| 299 | 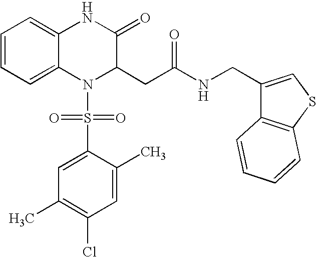 | 554.09 | 93 | MS(ESIpos): m/z = 554 (M + H) HPLC: Rt =4.95 min | C |
| 300 | 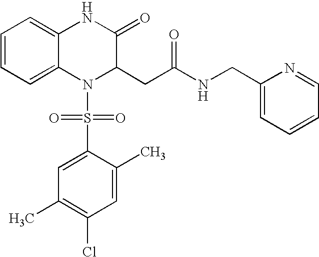 | 498.99 | 95 | MS(ESIpos): m/z = 499 (M + H) HPLC: Rt =4.09 min | C |
| 301 | 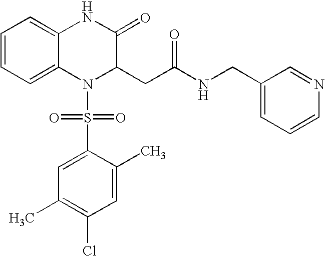 | 498.99 | 92 | MS(ESIpos): m/z = 499 (M + H) HPLC: Rt =4.03 min | C |
| 302 | 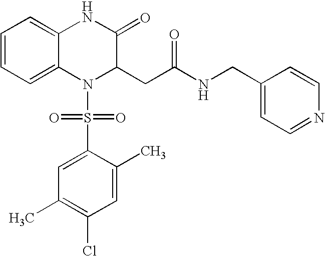 | 498.99 | 93 | MS(ESIpos): m/z = 499 (M + H) HPLC: Rt =4.09 min | C |
| 303 | 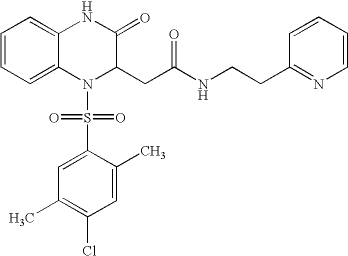 | 513.02 | 94 | MS(ESIpos): m/z = 513 (M + H) HPLC: Rt =4.01 min | C |
| 304 | 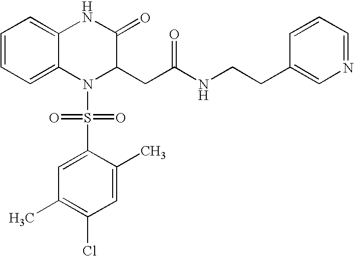 | 513.02 | 89 | MS(ESIpos): m/z = 513 (M + H) HPLC: Rt =4.05 min | C |
| 305 | 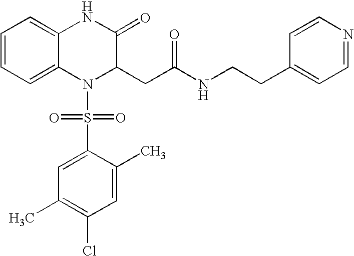 | 513.02 | 87 | MS(ESIpos): m/z = 513 (M + H) HPLC: Rt =4.04 min | C |
| 306 | 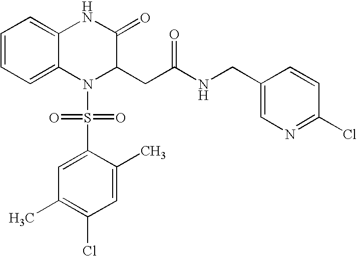 | 533.43 | 92 | MS(ESIpos): m/z = 533 (M + H) HPLC: Rt =4.52 min | C |
| 307 | 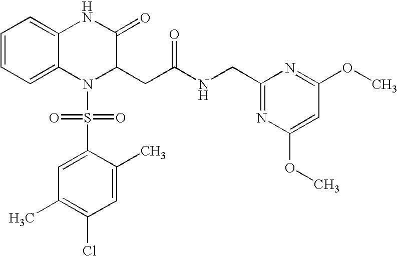 | 560.03 | 94 | MS(ESIpos): m/z = 560 (M + H) HPLC: Rt =4.58 min | C |
| 308 | 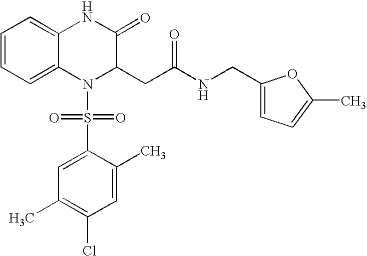 | 501.99 | 94 | MS(ESIpos): m/z = 502 (M + H) HPLC: Rt =4.68 min | C |
| 309 | 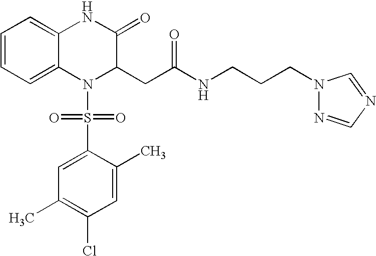 | 517.01 | 97 | MS(ESIpos): m/z = 517 (M + H) HPLC: Rt =4.07 min | C |
| 310 | 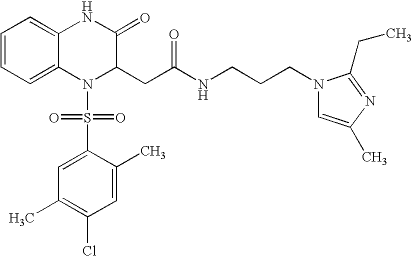 | 558.10 | 48 | MS(ESIpos): m/z = 558 (M + H) HPLC: Rt =4.23 min | C |

1. A compound of the formula (I) or (Ia), 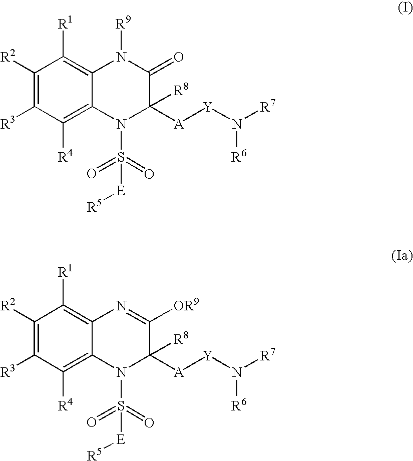
in which
A is (C₁-C₆)-alkanediyl,
E is a bond or (C₁-C₆)-alkanediyl,
Y is CO or SO₂,
R¹, R², R³ and R⁴ are identical or different and are hydrogen, halogen, trifluoromethyl, trifluoromethoxy, hydroxyl, nitro, cyano, amino, (C₁-C₆)-alkyl, (C₁-C₆)-alkoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl or carboxyl,
R⁵ is (C₆-C₁₀)-aryl or 5- to 10-membered heteroaryl, where aryl and heteroaryl are optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,
in which phenoxy, phenyl and 5- to 6-membered heteroaryl are optionally substituted identically or differently by trifluoromethyl, (C₁-C₆)-alkyl, (C₁-C₆)-alkoxy or halogen,
R⁶ and R⁷ are identical or different and
are hydrogen, (C₆-C₁₀)-aryl, 5- to 10-membered heteroaryl, 3- to 12-membered carbocyclyl, 4- to 12-heterocyclyl, or
are (C₁-C₁₀)-alkyl which is optionally substituted by halogen or a radical selected from the group of (C₁-C₆)-alkoxy, (C₆-C₁₀)-aryl, 5- to 10-membered heteroaryl, 3- to 12-membered carbocyclyl and 4- to 12-membered heterocyclyl,
where aryl, heteroaryl, heterocyclyl and carbocyclyl are optionally substituted identically or differently by radicals selected from the group of halogen, trifluoromethyl, trifluoromethoxy, hydroxyl, nitro, cyano, amino, (C₁-C₆)-alkyl, (C₃-C₈)-cycloalkyl, 5- to 7-membered heterocyclyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,
or
R⁶ and R⁷ together with the nitrogen atom form a 4- to 12-membered heterocyclyl radical which is bonded via nitrogen and which is optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, mono- or di-(C₁-C₆)-alkylamino, (C₁-C₆)-acyloxy, (C₁-C₆)-acyl, (C₁-C₆)-acylamino, (C₁-C₆)-alkoxycarbonyl, mono- or di-(C₁-C₆)-alkylaminocarbonyl, carbamoyl, carboxyl, (C₃-C₈)-Cycloalkyl and phenyl,
where alkyl, cycloalkyl and phenyl are optionally substituted identically or differently by radicals selected from the group of halogen, phenyl, (C₁-C₆)-alkyl, (C₁-C₆)-alkoxy and (C₁-C₆)-alkylthio, in which phenyl in turn is optionally substituted identically or differently by radicals selected from the group of halogen or methyl,
R⁸ is hydrogen or (C₁-C₃)-alkyl which is optionally substituted by fluorine,
R⁹ is hydrogen or (C₁-C₆)-alkyl,
and the salts, hydrates and/or solvates thereof,
with the exception of 2-[3-oxo-1-(phenylsulfonyl)-1,2,3,4-tetrahydro-2-quinoxalinyl]-N-phenylacetamide.

2. The compound as claimed in claim 1, where
A is (C₁-C₆)-alkanediyl,
E is a bond or (C₁-C₆)-alkanediyl,
Y is CO,
R¹, R², R³ and R⁴ are identical or different and are hydrogen, halogen, trifluoromethyl, trifluoromethoxy, hydroxyl, nitro, cyano, amino, (C₁-C₆)-alkyl, (C₁-C₆)-alkoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl or carboxyl,
R⁵ is (C₆-C₁₀)-aryl or 5- to 10-membered heteroaryl, where aryl and heteroaryl are optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,
in which phenoxy, phenyl and 5- to 6-membered heteroaryl are in turn optionally substituted identically or differently by trifluoromethyl, (C₁-C₆)-alkyl, (C₁-C₆)-alkoxy or halogen,
R⁶ and R⁷ are identical or different and are hydrogen, (C₆-C₁₀)-aryl, 5- to 10-membered heteroaryl, 3- to 12-membered carbocyclyl, 4- to 12-membered heterocyclyl, or
are (C₁-C₁₀)-alkyl which is optionally substituted by halogen or a radical selected from the group of (C₁-C₆)-alkoxy, (C₆-C₁₀)-aryl, 5- to 10-membered heteroaryl, 3- to 12-membered carbocyclyl and 4- to 12-membered heterocyclyl,
where aryl, heteroaryl, heterocyclyl and carbocyclyl are optionally substituted identically or differently by radicals selected from the group of halogen, trifluoromethyl, trifluoromethoxy, hydroxyl, nitro, cyano, amino, (C₁-C₆)-alkyl, (C₃-C₈)-cycloalkyl, 5- to 7-membered heterocyclyl, (C₁-C₆)-alkoxy, phenoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆)-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,
or
R⁶ and R⁷ together with the nitrogen atom form a 4- to 12-membered heterocyclyl radical which is bonded via nitrogen and which is optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, mono- or di-(C₁-C₆)-alkylamino, (C₁-C₆)-acyloxy, (C₁-C₆)-acyl, (C₁-C₆)-acylamino, (C₁-C₆)-alkoxycarbonyl, mono- or di-(C₁-C₆)-alkylaminocarbonyl, carbamoyl, carboxyl, (C₃-C₈)-cycloalkyl and phenyl,
where alkyl, cycloalkyl and phenyl in turn are optionally substituted identically or differently by one to three radicals selected from the group of halogen, phenyl, (C₁-C₆)-alkyl, (C₁-C₆)-alkoxy and (C₁-C₆)-alkylthio, in which phenyl in turn is optionally substituted identically or differently by radicals selected from the group of halogen or methyl,
R⁸ is hydrogen,
R⁹ is hydrogen,
and the salts, hydrates and/or solvates thereof,
with the exception of 2-[3-oxo-1-(phenylsulfonyl)-1,2,3,4-tetrahydro-2-quinoxalinyl]-N-phenylacetamide.

3. The compound as claimed in claim 1, where
A is methylene,
E is a bond,
Y is CO,
R¹, R², R³ and R⁴are identical or different and are hydrogen or halogen,
R⁵ is phenyl which is optionally substituted identically or differently by one to three radicals selected from the group of methyl, isopropyl, halogen, trifluoromethyl and trifluoromethoxy,
R⁶ and R⁷ are identical or different and are hydrogen, (C₁-C₆)-alkyl, phenyl or 5- to 8-membered carbocyclyl, where R⁶ and R⁷ are not both hydrogen, and where carbocyclyl and phenyl is optionally substituted identically or differently by radicals selected from the group of halogen, trifluoromethyl, trifluoromethoxy, methyl and methoxy,
R⁸ is hydrogen,
R⁹ is hydrogen,
and the salts, hydrates and/or solvates thereof,
with the exception of 2-[3-oxo-1-(phenylsulfonyl)-1,2,3,4-tetrahydro-2quinoxalinyl]-N-phenylacetamide.

4. A process for preparing compounds of the formula (I) as claimed in claim 1, wherein
[A] compounds of the general formula (II) or (IIa), 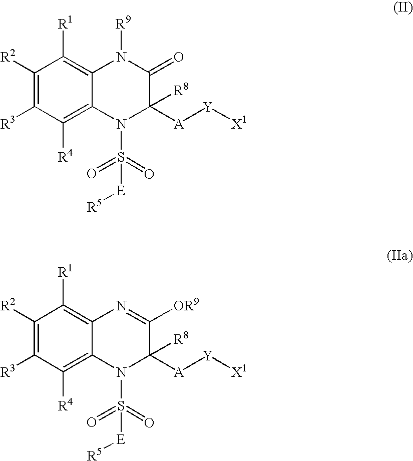
in which
A, E, Y, R¹, R², R³, R⁴, R⁵, R⁸ and R⁹ have the meaning indicated in claim 1, and
X¹ is halogen or hydroxyl,
are reacted with compounds of the general formula (III) 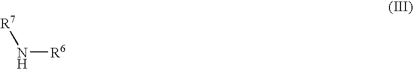
in which
R⁶ and R⁷ have the meaning indicated in claim 1,
or the salts thereof,
in inert solvents, where appropriate in the presence of a base and where appropriate in the presence of condensing agents,
or
[C] compounds of the general formula (V), 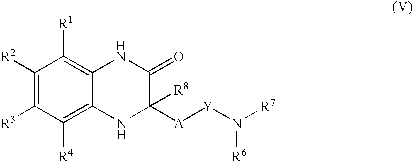
in which
A, Y, R¹, R², R³, R⁴, R⁶, R⁷ and R⁸ have the meaning indicated in claim 1,
are reacted with compounds of the general formula (VI) 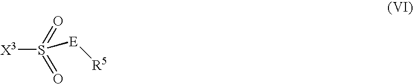
in which
E and R⁵ have the meaning indicated in claim 1, and
X³ is halogen,
in inert solvents, where appropriate in the presence of a base.

5. A compound of the formula (V) 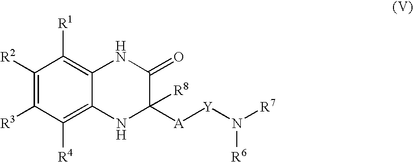
in which
A is (C₁-C₆)-alkanediyl,
Y is CO or SO₂,
R¹, R², R³ and R⁴ are identical or different and are hvdrogen, halogen, trifluoromethyl, trifluoromethoxy, hydroxyl, nitro, cyano, amino, (C₁-₆)-alkyl, (C₁-C₆)-alkoxy, (C₁-C₆)-alkylthio, mono- or di-(C₁-C₆)-alkylamino, (C₁-C₆)-acyl, (C₁-C₆)-acyloxy, (C₁-C₆)-acylamino, (C₁-C₆)-alkoxycarbonyl, mono- or di-(C₁-C₆-alkylaminocarbonyl, carbamoyl or carboxyl,
R⁶ and R⁷ are identical or different and
are hydrogen, (C₆-C₁₀)-aryl, 5- to 10-membered heteroaryl, 3- to 12-membered carbocyclyl, 4- to 12- heterocyclyl, or
are (C₁-C₁₀)-alkyl which is optionally substituted by halogen or a radical selected from the group of (C₁-C₆)-alkoxy, (C₆-C₁₀)-aryl, 5- to 10-membered heteroaryl, 3- to 12-membered carbocyclyl and 4- to 12-membered heterocyclyl,
where aryl, heteroaryl, heterocyclyl and carbocyclyl are optionally substituted identically or differently by radicals selected from the group of halogen, trifluoromethyl, trifluoromethoxy, hydroxyl, nitro, cyano, amino, (C₁-C₆)-alkyl, (C₃-C₈)-cycloalkyl, 5- to 7-membered heterocyclyl, (C₁-C₆)-alkoxy, phenoxy, (C₁-C₆)-alkylthio, mono- or di-(C₁-C₆)-alkylamino, (C₁-₆)-acyl, (C₁-C₆)-acyloxy, (C₁-C₆)-acylamino, (C₁-C₆)-alkoxycarbonyl, mono- or di-(C₁-C₆-alkylaminocarbonyl, carbamoyl, carboxyl, phenyl, 5- to 6-membered heteroaryl, propane-1,3-diyl, butane-1,4-diyl, 1,3-dioxapropane-1,3-diyl or 1,4-dioxabutane-1,4-diyl,
or
R⁶ and R⁷ together with the nitrogen atom form a 4- to 12-membered heterocyclyl radical which is bonded via nitrogen and which is optionally substituted identically or differently by radicals selected from the group of halogen, trifluoromethyl, trifluoromethoxy, hydroxyl, nitro, cyano, amino, (C₁-C₆)-alkyl, (C₁-C₆)-alkoxy, mono- or di-(C₁-C₆)-alkylamino, (C₁-C₆)-acyloxy, (C₁-C₆)-acyl, (C₁-C₆)-acylamino, (C₁-C₆)-alkoxycarbonyl, mono- or di-(C₁-C₆)-alkylaminocarbonyl, carbamoyl, carboxyl, (C₃-C₈)-cycloalkyl and phenyl,
where alkyl, cycloalkyl and phenyl are optionally substituted identically or differently by radicals selected from the group of halogen, phenyl, (C₁-₆)-alkyl, (C₁-C₆)-alkoxy and (C₁-C₆)-alkylthio, in which phenyl in turn is optionally substituted identically or differently by radicals selected from the group of halogen or methyl,
R⁸ is hydrogen or (C₁-C₃)-alkyl which is optionally substituted by fluorine,
and the salts, hydrates and/or solvates thereof.

6. (Cancelled).

7. A pharmaceutical composition comprising one or more of the compounds as claimed in claim 1 mixed together with one or more pharmaceutically suitable, essentially nontoxic carrier or excipient.

8. A method for the treatment and/or prophylaxis of states of pain comprising administering to a subject in need thereof an effective amount of one or more compounds of claim 1.

9. (Cancelled).
